(19) United States
(12) Patent Application Publication
(10) Pub. No.: US 2024/0175865 A1
(43) Pub. Date: May 30, 2024

(54) IMPROVED PROCESSES FOR RATIONALLY-DESIGNING AND PRODUCING BIOMOLECULES

(71) Applicants: Hans-Joachim WIEDEN, Winnipeg (CA); Dustin D. SMITH, West Lethbridge, (CA); Dylan GIRODAT, Los Alamos, NM (US); Luc Arden Hamilton ROBERTS, West Lethbridge (CA); Her Majesty The Queen in Right of Canada, as represented by the Minister of Agriculture and Agri-Foo, Lethbridge (CA)

(72) Inventors: Hans-Joachim WIEDEN, Winnipeg (CA); Dustin D. SMITH, Lethbridge (CA); Dylan GIRODAT, Los Alamos, NM (US); Luc Arden Hamilton ROBERTS, Lethbridge (CA); D. Wade ABBOT, Lethbridge (CA)

(21) Appl. No.: 17/772,238

(22) PCT Filed: Jan. 30, 2021
(86) PCT No.: PCT/CA2021/050110

§ 371 Date: Apr. 27, 2022

Related U.S. Application Data
(60) Provisional application No. 62/969,317, filed on Feb. 3, 2020.

(51) Int. Cl. G01N 33/53; C07K 14/24; C07K 14/245; C07K 14/315; G01N 33/533; G01N 33/542; G16B 15/30

(52) U.S. Cl. CPC G01N 33/5308; C07K 14/24; C07K 14/245; C07K 14/3156; G01N 33/533; G01N 33/542; G16B 15/30; C07K 2319/60; G01N 2333/24; G01N 2333/3156

(57) ABSTRACT

Improved processes for rationally-designing and producing biomolecules are disclosed herein. More specifically, disclosed processes comprise the steps of (i) selecting at least one biomolecule suitable for modification; (ii) obtaining at least one structure of the at least one biomolecule; (iii) simulating the molecular dynamics of the at least one structure to generate dynamic information about at least one position within the at least one structure; (iv) using the dynamic information to calculate a score for the at least one position; (v) comparing the score with at least one reference score to identify at least one target position within the biomolecule suitable for modification; and (vi) modifying the at least one target position. As proof-of-concept, the disclosed processes were used to rationally-design and produce maltooligosaccharide-detecting biosensors based on Streptococcus pneumoniae MalX, homogalacturonan breakdown product-detecting biosensors based on Yersinia enterocolitica TogB, and biosensors for observing conformational changes based on Escherichia coli EF-Tu.

### CROSS-REFERENCE TO RELATED APPLICATIONS

This application is a national stage application under 35 U.S.C. 371 and claims the benefit of PCT Application No. PCT/CA2021/050110 having an international filing date of Jan. 30, 2021, which designated the United States, which PCT application claimed the benefit of priority application U.S. Provisional Application No. 62/969,317 filed Feb. 3, 2020, the disclosures of each of which are hereby incorporated herein in their entireties by reference.

### REFERENCE TO SEQUENCE LISTING

This application contains a Sequence Listing submitted as an electronic text file named “10163HMQ-1-PUS-Seq_listing_ST25.txt”, having a size in bytes of 12,000 bytes, and created on Oct. 20, 2022. The information contained in this electronic file is hereby incorporated by reference in its entirety pursuant to 37 CFR § 1.52(e)(5).

### TECHNICAL FIELD

Embodiments herein relate generally to rationally-designed biomolecules and, specifically, to improved processes for rationally-designing and producing said biomolecules.

### BACKGROUND

Processes for designing and producing biomolecules are well known in the art. Such processes are used to create variants of naturally-occurring biomolecules or entirely synthetic biomolecules that have new or improved functions. These new or improved functions are enabled by the structure of the variant, which is modified compared to the structure of the original biomolecule.

Not all biomolecules are suitable for modification to create a useful variant. Suitable biomolecules may include polypeptides, nucleic acids, lipids, carbohydrates, and combinations thereof. Such biomolecules are generally characterized by a dynamic structure-function relationship, in which the physical (i.e. atomic and molecular composition), chemical (i.e. reactivity), and three-dimensional structure of the biomolecule determine how it interacts with its environment.

The term “polypeptide” is used herein to refer to any molecule comprised of one or more amino acid residues including peptides, dipeptides, oligopeptides, proteins, protein subunits or domains, and protein complexes, regardless of whether such amino acid residues are naturally-occurring, synthetic, or any combination thereof. In particular, it will be understood that such polypeptides may comprise any combination of stereoisomers including L-amino acids and D-amino acids. It will be further understood that such polypeptides may be produced through biological means, including cellular expression systems and cell-free expression systems, or through chemical synthesis means.

The term “nucleic acid” is used herein to refer to any molecule comprised of one or more nucleotides including deoxyribonucleic acids, ribonucleic acids, nucleic acid complexes, and ribozymes, regardless of whether such nucleotides are naturally-occurring, synthetic, or any combination thereof. In particular, it will be understood that such nucleic acids may comprise any combination of stereoisomers including D-nucleic acids and L-nucleic acids, as well as nucleic acids with varied configurations including locked nucleic acids. It will be further understood that such nucleic acids may be produced through biological means, including cellular expression systems and cell-free expression systems, or through chemical synthesis means.

The term “lipid” is used herein to refer to any molecule comprised of one or more fatty acids, glycerolipids, phospholipids, sphingolipids, sterols, prenols, saccharolipids, and polyketides, regardless of whether such lipids are naturally-occurring, synthetic, or any combination thereof. It will be understood that such lipids may be produced through biological means, including cellular expression systems and cell-free expression systems, or through chemical synthesis means.

The term “carbohydrate” is used herein to refer to any molecule comprised of one or more monosaccharides, disaccharides, oligosaccharides, and polysaccharides, regardless of whether such carbohydrates are naturally-occurring, synthetic, or any combination thereof. It will be understood that such carbohydrates may be produced through biological means, including cellular expression systems and cell-free expression systems, or through chemical synthesis means.

Many interactions between a biomolecule and its environment comprise the association and/or dissociation of a ligand from the biomolecule. The term “ligand” is used herein to refer to any compound that associates with a particular biomolecule. As such, a ligand may include a metal ion or a small molecule, however, it may also include another biomolecule or a complex of biomolecules depending on the circumstances. A ligand may also include other compositions of matter, including substrates, matrixes, constructs, and apparatuses, regardless of their size, solubility, or mobility. The result of such association and/or dissociation of a ligand from a biomolecule may be to change the structure of the biomolecule, change the structure of the ligand, change the characteristics of the environment (i.e. alter pH or ionic strength of a solvent), or any combination thereof. Such interactions may also absorb or emit energy including electromagnetic radiation (i.e. heat and light) and nuclear radiation (i.e. subatomic particles). These results generally comprise the “function” of the biomolecule within a particular system.

The ability of a biomolecule to produce a particular result and, therefore, serve a particular function is determined by the physical, chemical, and three-dimensional structure of the biomolecule. These structural features control, for example, what compounds may be ligands and how those ligands may be oriented relative to the biomolecule (i.e. a lock and key model), as well as the rate that such ligands may associate and dissociate from the biomolecule. These structural features also control how interactions between the biomolecule and its environment result in structural changes within the biomolecule and/or within the ligand, changes in the characteristics of the environment, the absorption and/or emission of specific forms of energy, and any combination thereof. It is understood that biomolecules may have structures and functions that are not directed to ligand binding or dissociation and that the examples provided herein are for illustration purposes only and are not exhaustive.

The relationship between biomolecule structure and function is generally well-known and understood. It is also generally well-known and understood that the function of a biomolecule can be altered by modifying the structure of the biomolecule. As such, processes have been developed to design and produce variants of biomolecules that demonstrate new or improved functions. These variants may be categorized according to the type of structural modification employed in each case. For instance, a large number of variants with industrial and research applications may be categorized according to four groups of structural modifications: addition of reporter groups, addition of linkers, intramolecular modifications, and addition of compounds for medical treatment, as are discussed in more detail below. It is appreciated that these four groups of structural modifications are not exhaustive nor mutually exclusive.

### Addition of Reporter Groups

Reporter groups are compounds (i.e. metal ions, molecules, or functional groups) that generate a signal or interfere with a signal when exposed to a particular stimulus. This stimulus-dependent behaviour may result in signal changes that can be reported by a suitable detector. In many cases, however, reporter groups are not effective at generating detectable signal changes on their own. Instead, reporter groups may require a biomolecule scaffold to mediate such detectable signal changes.

Modifying the structure of a biomolecule by adding one or more reporter group may cause the biomolecule-reporter group complex (i.e. the variant) to exhibit biosensing functionality and, thus, become a biosensor. Biosensors are typically used to detect the presence of one or more analytes (i.e. ligands of interest) and/or quantify the concentration of one or more analytes in a solution (MEHROTRA, P. Biosensors And Their Applications—A Review. Journal of Oral Biology and Craniofacial Research, 6 (2016) 153-159). As used herein, the term “analyte” can include naturally-occurring and/or synthetic compounds including metal ions, small molecules, pharmaceutical compounds of medical treatment (e.g. drugs and therapeutics), biomolecules (e.g. polypeptides, nucleic acids, lipids, and carbohydrates), biomolecule complexes, organelles, cells, tissues, and combinations thereof. Although some of the examples that follow are directed towards biosensors designed to detect and quantify such analytes, it will be appreciated that biosensors may be used to detect and quantify other physical and chemical stimuli including temperature, pH, and/or ionic strength. It will be further appreciated that biosensors may be used for other research and industrial processes (e.g. studies directed to understanding conformational changes or the transmission of dynamic information within a biomolecule of interest).

The market value for biosensors in 2019 was 21.1 billion USD and is projected to exceed 30 billion USD by 2024 (MARKETSANDMARKETS. Biosensors Market by Type (Sensor patch and embedded device), Product (Wearable and nonwearable), Technology (Electrochemical and optical), Application (POC, Home Diagnostics, Research Lab, Food & Beverages), and Geography—Global Forecast to 2024. May 2019. SE3097). These projections are derived from the impact that biosensors have on, but are not limited to, medicine, agriculture, environmental monitoring, food and beverage production, biofuels, and academic research (HARRISON M., DUNLOP, M. Synthetic Feedback Loop Model for Increasing Microbial Biofuel Production using a Biosensor. Frontiers in Microbiology, 3 (2012) 360; HELLER, A., FELDMAN, B. Electrochemical Glucose Sensors and their Applications in Diabetes Management. Chemical Reviews, 108 (2008) 2482-2505; HUGHES, M. D. The Business of Self-Monitoring of Blood Glucose: A Market Profile. Journal of Diabetes Science and Technology, 3 (2009) 1219-1223; ISPAS, C. R., CRIVAT, G., ANDREESCU, S. Recent Developments in Enzyme-Based Biosensors for Biomedical Analysis. Analytical Letters, 45 (2012) 168-186; MEHROTRA, P. Biosensors and Their Applications—A Review. Journal of Oral Biology and Craniofacial Research, 6 (2016) 153-159; PARDEE, K., et al., Paper-Based Synthetic Gene Networks. Cell, 159 (2014) 940-954; PARDEE, K., et al., Rapid, Low-Cost Detection of Zika Virus using Programmable Biomolecular Components. Cell, 165 (2016) 1255-1266; RAI, V., ACHARYA, S., DEY, N., Implications of Nanobiosensors in Agriculture. Journal of Biomaterials and Nanobiotechnology, 3 (2012) 315; SHIN, H. J., Genetically Engineered Microbial Biosensors for in Situ Monitoring of Environmental Pollution. Applied Microbiology and Biotechnology, 89 (2011) 867-877; SRILATHA, B., Nanotechnology in Agriculture. Journal of Nanomedicine and Nanotechnology, 2 (2011); VELASCO-GARCIA, M. N., MOTTRAM, T., Biosensor Technology Addressing Agricultural Problems. Biosystems Engineering, 84 (2003) 1-12). As such, there is a need to design and produce novel biosensors for a wide-range of industrial and research applications. In particular, there is a need to design and produce novel biosensors that are capable of specific and sensitive monitoring of analytes at low concentrations and with high dynamic range. There is also a need to design and produce biosensors that can be used to understand conformational changes within a biomolecule of interest.

As a specific example, there is a need to develop novel biosensors that can detect and quantify carbohydrate analytes within diverse solutions. Carbohydrate active enzymes (CAZymes) are a group of sequence-diverse enzyme families belonging to five different functional classes that modify the linkages or decorations of carbohydrates (LOMBARD, V., et al., The Carbohydrate-Active Enzymes Database (CAZy) in 2013. Nucleic Acids Research, 42 (2013) D490-D495). CAZymes are commonly used in bioindustrial processes, including biofuel production, food and beverage processing, and textile finishing (POLAINA, J., MACCABE, A. P., Industrial Enzymes. Springer 2007). To determine substrate specificity and kinetics of a CAZyme, a suitable assay must be developed. Commonly, activity assays can be laborious, involve non-continuous steps (i.e. require manual time points), non-specific (i.e. detect many carbohydrates at once) or are linked (i.e. depend on other enzymes or cofactors) and don't lend themselves well to high-throughput or multiplexing. Biosensors capable of specifically detecting and measuring the concentration of carbohydrate substrates or products of CAZyme-catalysed reactions could find application in such assays. Examples of such products of CAZyme-catalyzed reactions include maltooligosaccharides (MOS) having a degree of polymerization of between three to eleven glucose residues and homogalacturonan breakdown products (HBP) including 4,5-unsaturated digalacturonic acid, digalacturonic acid, and trigalacturonic acid.

As another specific example, there is a need to develop novel biosensors that can be used to observe conformational changes within prokaryotic elongation factor thermo unstable (EF-Tu) and its eukaryotic and archaeal homologs such as elongation factor thermo unstable, mitochondrial (TUFM) and the alpha subunit of the eukaryotic elongation factor (eEF-1A). EF-Tu is one of the most abundant and highly conserved proteins in prokaryotes given its crucial role in translation. EF-Tu is a guanine nucleotide-binding protein (G-protein) responsible for catalyzing the binding of aminoacyl-tRNA (aa-tRNA) to ribosomes. EF-Tu is predicted to undergo a number of conformational changes during its active cycle, which are classically associated with its various ligand-bound states. Specifically, EF-Tu is predicted to adopt at least one first conformation while bound to guanosine diphosphate (GDP) and at least one second conformation while bound to guanosine triphosphate (GTP). The various conformational changes undergone by EF-Tu are not well-understood and, in particular, the effect that such conformational changes have on limiting ligand dissociation from EF-Tu remains to be elucidated. Studying these conformational changes is a critical step towards, for example, designing and producing novel antibiotics that target EF-Tu. Biosensors capable of providing insight into the mechanisms involved in EF-Tu's structural rearrangements could find application in such studies.

It will be appreciated that the above-listed examples are not exhaustive and are provided for illustration purposes only.

### Addition of Linkers

Linkers are compounds that may be used to mediate reversable or irreversible interactions between at least one biomolecule and at least one target ligand. Such interactions may comprise highly-specific covalent or non-covalent conjugation between the linker and at least one functional group of the biomolecule as well as between the linker and at least one functional group of the target ligand. Linkers may comprise a metal ion or small molecule (including functional groups) that enable the direct binding of the biomolecule to the ligand. In other instances, linkers may comprise a single residue or subunit of a biomolecule (e.g. amino acid, nucleotide, sugar, etc.) that mediate interactions between the biomolecule and ligand. Linkers may also comprise two or more residues or subunits that mediate interactions between the biomolecule and ligand. In some configurations, these two or more residues or subunits may be cleaved from one another with high specificity in order to sever the interaction between the biomolecule and the target ligand. Further variations in ligand composition, configuration, and uses are known.

The need to design and produce novel biomolecule variants that can be bound or localized to a particular ligand is apparent. In some circumstances, a target ligand may be an immobile substrate that comprises part of an apparatus. For instance, it may be desirable to localize a biomolecule within a microfluidic device. A solution may then be pumped through the device, where it interacts with the localized biomolecule without causing the biomolecule to become dissolved within the solution. Applications for these apparatuses include multiplexed diagnostic devices for detecting several analytes within a solution. In such applications, a set of biosensors may be localized at pre-determined positions within the device. The addition of a different linker to each species of biosensor may cause each species to bind to one substrate and not another. Different types of substrates may then be positioned throughout the device, which can be used to localize each species of biosensor to a particular position within the device. The benefit of such devices is that the signal generated by each species of biosensor is, therefore, also localized to a particular position within the device. This localization reduces interference between signals generated by different species of biomolecule. As the solution is pumped through the device, each analyte dissolved within the solution will cause its complementary species of biosensor to emit a detectable signal change at a different position within the device. The different positions can be monitored independently by one or more detectors to detect specific signal changes associated with the presence of more than one analyte dissolved within the solution. Such devices are often used as a diagnostic tool for complex solutions such as blood.

It is appreciated that such apparatuses may have other configurations and uses. For example, similar apparatuses may be used in methods for purifying compounds. In such applications, a biomolecule that reversibly binds to a ligand of interest may be localized on a substrate within a device (e.g. a resin matrix) via a suitable linker. As a first solution comprising the ligand is introduced to the matrix, the ligand will bind to the localized biomolecule while the first solution and other contaminants dissolved or suspended therein are discarded. A second solution may then be introduced to the matrix, which induces the dissociation of the target ligand from the localized biomolecule. After the target ligand has become dissolved or suspended, the second solution comprising the purified target ligand may be collected for a variety of downstream applications. Such apparatuses and methods of use emphasize the need to design and produce novel biomolecules that are modified by the addition of a linker.

Adding a linker to a biomolecule may be desirable for yet other applications. For example, in circumstances where the biomolecule may be a drug or therapeutic for medical treatment and a target ligand may be a drug delivery system such as a vesicle, adding a linker to the biomolecule may permit the biomolecule to bind to the drug delivery system, whereby it can be transported to a treatment site or tissue within a patent. Such methods of medical treatment further emphasize the need to design and produce novel biomolecules that are modified by the addition of a linker.

It is appreciated that the above-listed examples are not exhaustive and are provided for illustration purposes only.

### Intramolecular Modifications

Intramolecular modifications may generally refer to additions, deletions, or substitutions of atoms, functional groups, residues, and/or subunits within a biomolecule. Such modifications may be made to a biomolecule to produce variants that are useful in structure-function studies or demonstrate an altered function.

In some instances, it may be desirable to add, subtract, or substitute such elements of a biomolecule in order to study how that element and biomolecule function within a particular environment. For example, in the case of polypeptides, a catalytic residue of an enzyme may be elucidated by introducing point mutations within the enzyme and detecting whether the resultant variants are catalytically active (see e.g. ROSLER K., MERCIER E., ANDREWS I., WIEDEN H. J., Histidine 114 is Critical for ATP Hydrolysis by the Universally Conserved ATPase YchF. The Journal of Biological Chemistry, 2015 Jul. 24; 290(30), pages 18650-61).

In yet other instances, it may be desirable to engineer a biomolecule variant that has an altered or entirely different function (see e.g. LAOS R., SHAW R., LEAL N. A., GAUCHER E., BENNER S., Directed Evolution of Polymerases to Accept Nucleotides with Nonstandard Hydrogen Bond Patterns. Biochemistry, 2013 Aug. 6; 52(31), pages 5288-94; GIVER, L., GERSHENSON, A., FRESKGARD, P., ARNOLD, F. H., Directed Evolution of a Thermostable Esterase. Proceedings of the National Academy of Sciences, 1998 October 95(22), pages 12809-13). For example, intramolecular modifications may be used to selectively stabilize one conformational state over another to affect biomolecule function within a particular system (see e.g. MARVIN J S, HELLINGA H W. Manipulation of ligand binding affinity by exploitation of conformational coupling. Nat. Struct. Biol. 2001 September; 8(9), pages 795-8).

It is appreciated that the above-listed examples are not exhaustive and are provided for illustration purposes only. The examples do emphasize a need to design and produce novel biomolecule variants that are modified by intramolecular additions, deletions, or substitutions.

### Addition of Compounds for Medical Treatment

Biomolecules are often involved in cellular functions that are critical to the survival and reproduction of an organism or a virus. Many medical treatments for bacterial infections, viral infections, autoimmune disorders, cancers, and the like comprise the specific targeting of critical biomolecules within a bacterium, virus, human cell, or the like. Compounds for medical treatment, including drugs and therapeutics, may interact with such critical biomolecules, thus, preventing or limiting cellular functions such as cell growth and cell division. For example, such drugs and therapeutics may irreversibly bind to critical biomolecules and thereby inhibit their function. In this context, the desired biomolecule variant may be a complex of the biomolecule and an inhibiting drug or therapeutic that has reduced functionality compared to the unmodified biomolecule.

It is appreciated that the above-listed example is not exhaustive and is provided for illustration purposes only. The example does emphasize the need to design and produce novel complexes between biomolecules and compounds for medical treatment.

### Known Design Processes

When referring to the “design” of biomolecules, this term generally comprises a mental process in which a researcher conceives of a variant of a particular biomolecule that may be useful for an intended purpose (such as the examples listed above). Once the variant is conceived, it may be produced through known processes. The variant must then be tested by known methods to confirm that it is useful for the intended purpose.

In such known processes for designing biomolecules, the conception step is based primarily on the researcher's judgment. The researcher must possess specialized expertise in order to make a “best guess” about how to modify the biomolecule to create a variant that is operable for the intended purpose. For example, in the case of designing a polypeptide-based biosensor, the researcher may intend for the biosensor to exhibit a detectable signal change when exposed to an analyte of interest. The researcher may use his or her knowledge of differences between the apo (i.e. unbound, open conformational state) and analyte-bound (ligand-bound, closed conformational state) structures of the polypeptide to select a labelling position for a reporter group that he or she predicts will experience an environmental change upon analyte binding or dissociation. This prediction requires not only detailed knowledge about the polypeptide in question but also an understanding of how to apply that information to select one labelling position over another. In such cases, target positions within the polypeptide may be described as allosteric, peristeric, and endosteric. Allosteric positions are located distally from the analyte binding site, yet still undergo local environmental changes as a result of analyte binding or dissociation. Peristeric positions are located adjacent to the analyte binding site. Endosteric positions are located within the analyte binding site. Development of biosensors often involve reporter group labelling at peristeric and endosteric positions to take advantage of the analyte binding event to induce an environmental change for the reporter group (e.g. BRUNE, M., et al., Direct, Real-Time Measurement of Rapid Inorganic Phosphate Release using a Novel Fluorescent Probe and its Application to Actomyosin Subfragment 1 ATPase. Biochemistry, 33 (1994), pages 8262-71; DE LORIMIER, R. M., et al., Construction of a Fluorescent Biosensor Family. Protein Science, 11 (2002), pages 2655-75; GILARDI, G., et al., Engineering the Maltose Binding Protein for Reagentless Fluorescence Sensing. Analytical Chemistry, 66 (1994), pages 3840-47). This is because it is generally easy to predict that the analyte binding site will undergo conformational changes upon analyte binding or dissociation. However, introducing a reporter group (or ligand) in close proximity to the analyte binding site can perturb the activity of the resulting biosensor-analyte conjugate, reducing their binding affinity or specificity for their cognate analyte (see e.g. BRUNE, M., et al., Direct, Real-Time Measurement of Rapid Inorganic Phosphate Release using a Novel Fluorescent Probe and its Application to Actomyosin Subfragment 1 ATPase. Biochemistry, 33 (1994), pages 8262-71; DE LORIMIER, R. M., et al., Construction of a Fluorescent Biosensor Family. Protein Science, 11 (2002), pages 2655-75; GILARDI, G., et al., Engineering the Maltose Binding Protein for Reagentless Fluorescence Sensing. Analytical Chemistry, 66 (1994), pages 3840-47; TOSELAND, C. P., Fluorescent Labeling and Modification of Proteins, Journal of Chemical Biology, 6 (2013), pages 85-95). As this example illustrates, modification of allosteric sites is generally preferable where it is undesirable to interfere with the interaction between the biomolecule and its ligand. Conversely, other circumstances are understood in which it may be desirable to perturb the ligand binding site through allosteric means (for example, through the addition of a compound for medical treatment). Unfortunately, allosteric sites are generally very difficult to predict in either circumstance.

Given the complexity of biomolecules, even the most well-informed “best guess” is unlikely to have a high success rate, particularly for allosteric sites. As such, the selection of target positions within a biomolecule that are suitable for modification is an essentially stochastic exercise with little more than a random chance at success. This limitation has resulted in the widespread adoption of a “shotgun” trial-and-error approach that requires many variants to be designed, produced, and tested before a useful variant is likely to be discovered. Such an approach is often time-consuming, expensive, and labour-intensive. These negative factors also have a tendency to compound exponentially as the complexity of the biomolecule and/or its desired functionality increases.

As such, processes have been developed to enable the rational design of biomolecules in an effort to increase success rates. The term “rational design” is used herein to refer to the process of generating a variant of a known biomolecule that can be reliably predicted to exhibit a particular, desired functionality. Instead of selecting a target position for modification based on subjective judgment and expertise—which is unlikely result in a variant with the desired functionality—a truly rational design process will provide an objective determination of target positions that are much more likely to result in a variant with the desired functionality. In order to achieve rational design, the relationship between the structural modification and the desired functionality must be understood and predicted reliably. It is this concept of predictability that differentiates a rational design process from a merely stochastic process.

Several known rational design processes are directed to a computational approach for selecting target positions within a biomolecule that are suitable for modification. The computational approach is intended to predict the suitability of target positions based on structural information about the biomolecule that may not otherwise be readily apparent.

Marvin et al., Proc. Natl. Acad. Sci. USA 94:4366-4371

One known process for rationally-designing polypeptides is disclosed by Marvin et al. Therein, the disclosed process comprises the steps of (i) selecting a suitable polypeptide for modification, (ii) obtaining a static apo and ligand-bound structure of the polypeptide, (iii) measuring the relative distance between C_{α} atoms within each static structure, and (iv) identifying regions of the polypeptide that have differences in the relative distance between Ca atoms between each static structure. The process is intended to narrow down the number of suitable target positions before proceeding with the known “shotgun” trial-and-error approach for the remaining suitable target positions. As a proof-of-concept, the process was used by Marvin et al. to design and produce variants of the Escherichia coli maltose-binding protein that are modified by environmentally-sensitive reporter groups. The target positions modified by the reporter groups were, at least in part, selected based on structural information generated using the disclosed process and the resultant predictions made by Marvin et al. about local conformational changes within the polypeptide. Specifically, the disclosed process was used to generate structural information about the relative distance between C_{α} atoms of Escherichia coli maltose-binding protein in an apo and maltose-bound state. The structural information was then used to identify regions of the protein that are likely to exhibit ligand-dependent changes in the relative distance between C_{α} atoms. These ligand-dependent changes, in turn, were used by Marvin et al. to predict regions of the biomolecule that were likely to undergo local conformational changes upon maltose binding or dissociation, which were further predicted to comprise specific positions that are suitable for modification by the environmentally-sensitive reporter group.

The process disclosed by Marvin et al. was successful in aiding researchers to select two target positions that were suitable for modification. The process was not without limitations, however. Most critically, the process was not truly non-stochastic. Eight regions of the Escherichia coli maltose-binding protein were identified following analysis of the structural information. Of these eight regions, six were false-positive identifications comprising target positions that were undesirably located within or adjacent to the ligand binding pocket or the partially-disordered N-terminus of the polypeptide. Significant judgment and expertise were required to discount these undesirable target regions.

The second most critical limitation of this process is that it does not provide a means for ranking the relative quality of individual target positions with an identified target region without further exercise of judgment and expertise. This limitation is likely to result in continued application of the “shotgun” trial-and-error approach discussed previously, which does not result in saved labour, expense, or time. Indeed, once target regions are identified, a researcher must use conventional methods to select individual target positions within those regions. This was the case for Marvin et al., who report that “any attempt at the prediction of locations with the highest response would require a detailed molecular simulation of the conformational ensembles of the fluorophores in the presence of solvent, a nontrivial proposition. We therefore constructed several different cysteine mutations in a given region to establish empirically which mutation gives the most pronounced changes.”

A third limitation of this process is that it cannot identify target positions within regions of the biomolecule that have transient or cryptic properties that exist in-between apo and/or ligand-bound states. Not all biomolecules exhibit structural differences in their apo and/or ligand-bound states that comprise suitable target positions for modification; instead, suitable target positions may be located elsewhere in the biomolecule where structural changes occur only temporarily between states. The process disclosed by Marvin et al. cannot be used to identify such transient or cryptic target positions as it considers only static structures.

A fourth limitation of this process is that it can only detect ligand-dependent changes within the biomolecule that are based on the relative distance between the C_{α} atoms of amino acid residues. Suitable target positions may be indicated by other factors, which are not disclosed or contemplated by Marvin et al. This consideration is particularly true for biomolecule variants that do not rely on conformational changes to perform their desired function or that are conformationally dynamic and can adopt more than two states. This consideration is also particularly true for non-polypeptide-based biomolecules that do no comprise amino acid residues with C_{α} atoms.

U.S. Pat. No. 10,060,920

Another known process for rationally-designing polypeptides is disclosed in U.S. Pat. No. 10,060,902 (the '920 patent). Therein, the disclosed process comprises the steps of (i) selecting a suitable polypeptide for modification, (ii) obtaining a static apo and at least one ligand-bound structure of the polypeptide, (iii) measuring the dihedral angles (defined by the C_{α} atoms spanning four residues) within each static structure, and (iv) identifying regions of the polypeptide that have differences in such dihedral angles between at least two static structures. The process is intended to narrow down the number of suitable target positions before proceeding with the known “shotgun” trial-and-error approach for the remaining suitable target positions. As a proof-of-concept, the process was used to design and produce variants of at least the Escherichia coli maltodextrin-binding protein that are modified by environmentally-sensitive reporter groups.

The target positions modified by the reporter groups were, at least in part, selected based on structural information generated using the disclosed process and the resultant predictions made about local conformational changes within the polypeptide. Specifically, the disclosed process was used to generate structural information about the dihedral angles within at least the Escherichia coli maltodextrin-binding protein in an apo and at least one ligand-bound state. The structural information was then used to identify groups of four sequentially-adjacent residues of the protein that are likely to exhibit ligand-dependent changes in dihedral angles. These ligand-dependent changes, in turn, were used to predict groups of four sequentially-adjacent residues within the biomolecule that undergo local conformational changes upon ligand binding or dissociation, which were further predicted to comprise specific positions that are suitable for modification by the environmentally-sensitive reporter group.

The '920 patent is an improvement over the process disclosed by Marvin et al., given that is not limited to identifying general regions of a polypeptide that are likely to comprise suitable target positions. Instead, the '920 patent discloses a process for identifying groups of four sequentially-adjacent positions that may comprise a suitable target position. Despite this improvement, however, the process disclosed in the '920 patent suffers from a number of limitations and is not truly non-stochastic.

A first limitation of this process is that it still requires judgment and expertise to discount identified groups of four sequentially-adjacent residues that are unlikely to comprise suitable target positions. Given that this process considers only changes in dihedral angles, it will detect groups of four sequentially-adjacent residues that may be undesirably located within or adjacent to the ligand binding pocket or a disordered region of the polypeptide. The process does not provide an objective means for discounting such identified groups.

A second limitation of this process is that it cannot be used to select individual target positions within an identified group of four sequentially-adjacent residues. It does not provide a means for ranking the relative quality of individual target positions without the exercise of judgment and expertise. This requirement will likely lead to continued use of the “shotgun” trial-and-error approach to identify individual target position that are suitable for modification (albeit with potentially fewer candidate variants).

A third limitation of this process is that it cannot identify target positions within regions of the biomolecule that have transient or cryptic properties that exist between apo and/or ligand-bound states. Not all biomolecules exhibit structural differences in their apo and/or ligand-bound states that comprise suitable target positions for modification; instead, suitable target positions may be located elsewhere in the biomolecule where structural changes occur only temporarily between states. The process disclosed in the '920 patent cannot be used to identify such transient or cryptic target positions as it considers only static structures.

A fourth limitation of this process is that it can only detect ligand-dependent changes within the biomolecule that are based on dihedral angles (defined by the C_{α} atoms spanning four amino acid residues) within each static structure. Suitable target positions may be indicated by other factors, which are not disclosed or contemplated in the '920 patent. This consideration is particularly true for biomolecule variants that do not rely on conformational changes to perform their desired function or that are conformationally dynamic and can adopt more than two states. This consideration is also particularly true for non-polypeptide-based biomolecules that do no comprise amino acid residues with C_{α} atoms.

### European Patent Application No. 2 103 936

Yet another known process for rationally-designing polypeptides is disclosed in European Patent Application Number 2 103 936 (the '936 patent application). Therein, the disclosed process comprises the steps of (i) selecting a suitable polypeptide for modification, (ii) obtaining a static apo and at least one ligand-bound structure of the polypeptide, (iii) measuring the solvent accessibility of each residue within each static structure, (iv) determining whether each residue is in contact with a ligand via a water molecule or does not contact the ligand; and (v) identifying residues of the polypeptide that have differences in solvent accessibility between at least two static structures and are located suitably close to or distant from the ligand. The process is intended to narrow down the number of suitable target positions before proceeding with the known “shotgun” trial-and-error approach for the remaining suitable target positions. As a proof-of-concept, the process was used to design and produce variants of the Designed Ankyrin Repeat Protein (Darpin) that are modified by environmentally-sensitive reporter groups.

The '936 patent application is an improvement over the processes disclosed by Marvin et al. and the '920 patent. Namely, the '936 patent discloses a method for identifying individual target positions that may be suitable for modification and, further, discloses a method for discounting undesirable target positions based on their distance to the ligand. Despite these improvements, however, the process disclosed in the '936 patent application suffers from a number of limitations and is not truly non-stochastic.

A first limitation of this process is that it still requires judgment and expertise to discount identified target positions that are unlikely to be suitable. Given that this process considers only changes in solvent accessibility and distance between target position and ligand, it may still identify target positions that may be undesirably located within, for example, a disordered region of the polypeptide. The process does not provide an objective means for discounting such identified target positions in all circumstances.

A second limitation of this process is that it does not provide a means for ranking the relative quality of individual target positions without the exercise of judgment and expertise. This requirement is likely to lead to continued use of the “shotgun” trial-and-error approach to identify individual target position that are suitable for modification (albeit with potentially fewer candidate variants).

A third limitation of this process is that it cannot identify target positions within regions of the biomolecule that have transient or cryptic properties that exist between apo and/or ligand-bound states. Not all biomolecules exhibit structural differences in their apo and/or ligand-bound states that comprise suitable target positions for modification; instead, suitable target positions may be located elsewhere in the biomolecule where structural changes occur only temporarily between states. The process disclosed in the '936 patent application cannot be used to identify such transient or cryptic target positions as it considers only static structures.

A fourth limitation of this process is that it can only detect ligand-dependent changes within the biomolecule that are based on solvent accessibility of amino acid residues within each static structure. Suitable target positions may be indicated by other factors, which are not disclosed or contemplated by the '936 patent application. This consideration is particularly true for biomolecule variants that do not rely on conformational changes to perform their desired function or that are conformationally dynamic and can adopt more than two states.

Considering the foregoing, there is a need for improved processes for rationally-designing and producing biomolecules. Specifically, there is a need for non-stochastic processes for selecting target positions within a biomolecule that are suitable for modification. Preferably, such non-stochastic processes will eliminate the need for judgment and expertise to be exercised in the selection of a suitable target position. Furthermore, preferred non-stochastic processes will incorporate a modular approach that allows target positions to be identified in all species of biomolecules for all potential modifications and all desired functionalities.

### SUMMARY OF INVENTION

According to the present embodiments, improved processes for rationally-designing and producing biomolecules are disclosed herein. More specifically, present processes may comprise the steps of (i) selecting at least one biomolecule suitable for modification, (ii) obtaining at least one structure of the at least one biomolecule, (iii) simulating the molecular dynamics of the at least one structure to generate dynamic information about at least one position within the at least one structure, (iv) using the dynamic information to calculate a score for the at least one position, (v) comparing the score with at least one reference score to identify at least one target position within the biomolecule suitable for modification, and (vi) modifying the at least one target position. As will be disclosed in more detail herein, present processes were used to rationally-design and produce MOS-detecting biosensors based on the Streptococcus pneumoniae MalX biomolecule, homogalacturonan breakdown product-detecting biosensors based on Yersinia enterocolitica TogB biomolecule, and biosensors for observing conformational changes based on Escherichia col EF-Tu biomolecule.

In some embodiments, present processes may be used to design and produce modified polypeptides, nucleic acids, lipids, or carbohydrates.

In some embodiments, the modifying of the at least one target position may comprise the addition of a reporter group. Such reporter groups may comprise a redox cofactor or a fluorophore.

In some embodiments, the modifying of the at least one target position may comprise the addition of a linker.

In some embodiments, the modifying of the at least one target position may comprise an intramolecular modification, such as an addition, deletion, or substitution. In the case of polypeptides, such intramolecular modifications may result in the introduction of a cysteine residue.

In some embodiments, the at least one structure may comprise a three-dimensional representation of the at least one biomolecule in an apo configuration or a ligand-bound configuration. Such structures may be obtained by a method such as crystallography, cryogenic electron microscopy (cryo-EM), nuclear magnetic resonance (NMR) spectroscopy, or electron paramagnetic resonance (EPR) spectroscopy. Such structures may also be obtained by prediction modelling.

In some embodiments, the score for the at least one position may be compared to a reference score for at least one other position within the at least one structure or a reference score that is pre-determined.

In some embodiments, the modified biomolecule when designed and produced by the present processes may be a biosensor for maltooligosaccharides that comprises a Streptococcus pneumoniae (S. pneumoniae) MalX polypeptide and at least one reporter group. Such maltooligosaccharides may have a degree of polymerization of between three to eleven glucose residues. The reporter group may be attached at one or more amino acid positions of the S. pneumoniae MalX polypeptide, for example, at amino acid position 128 or 243. The reporter group may be attached covalently or non-covalently. The S. pneumoniae MalX polypeptide may be further modified by intramolecular modifications, thereby creating, for example, an A128C or T243C variant.

In some embodiments, the modified biomolecule when designed and produced by the present processes may be a biosensor for homogalacturonan breakdown products that comprises a Yersinia enterocolitica (Y. enterocolitica) TogB polypeptide and at least one reporter group. Such homogalacturonan breakdown products may be 4,5-unsaturated digalacturonic acid, digalacturonic acid, and trigalacturonic acid. The reporter group may be attached at one or more amino acid positions of the Y. enterocolitica TogB polypeptide, for example, at amino acid position 242, 279, 357, or 358. The reporter group may be attached covalently or non-covalently. The S. pneumoniae MalX polypeptide may be further modified by intramolecular modifications, thereby creating, for example, an F242C, A279C, K357C, or D358C variant.

In some embodiments, the modified biomolecule when designed and produced by the present processes may be a biosensor for conformational changes that comprises an Escherichia coli (E. coli) EF-Tu polypeptide and at least one reporter group. The reporter group may be attached at one or more amino acid positions of the E. coli EF-Tu polypeptide, for example, at amino acid position 202 or 265. The reporter group may be attached covalently or non-covalently. The S. pneumoniae MalX polypeptide may be further modified by intramolecular modifications, thereby creating, for example, a T34C E202C or T34C L265C variant.

In some embodiments, the modified biomolecule when designed and produced by the present processes may be modified by the addition of at least one reporter group that is a redox cofactor or a fluorophore. In the case of a fluorophore, the fluorophore may be a member of the naphthalene family, a member of the xanthene family, and a member of the pyrene family. More specifically, the fluorophore may be 7-diethylamino-3-(4′-maleimidylphenyl)-4-methylcoumarin (CPM), 7-diethylamino-3-[N-(2-maleimidoethyl)carbamoyl]coumarin (MDCC), N-(7-dimethylamino-4-methylcoumarin-3-yl)maleimide (DACM), N-[2-(dansylamino)ethyl]maleimide (Dansyl), fluorescein-5-maleimide (Fluorescein), N-(1-pyrene)maleimide (Pyrene), Rhodamine Red C2 maleimide (Rhodamine Red), and 5-(2-iodoacetylaminoethyl)aminonaphthalene-1-sulfonic acid (IAEDANS).

As will be appreciated, present processes are non-stochastic and directed to eliminating the need for judgment and expertise to be exercised in the selection of target positions within a biomolecule that are suitable for modification. Present processes are further contemplated to be operable for a wide-range of biomolecules, modifications, and desired functionalities. In particular, present processes can be used to identify suitable target positions within biomolecules that are transient or cryptic. Present processes have high success rates compared to known processes and are, thus, able to reduce costs and expedite biomolecule design and production. Such outcomes are enabled, in part, by the ability of present processes to consider any number of factors that are known to affect the structure of a biomolecule in an intuitive and modular way. Other features and advantages of present processes will be apparent from the following detailed description, drawings, and claims.

### BRIEF DESCRIPTION OF THE DRAWINGS

FIGS. 1-20 show various structures and results related to the presently-disclosed process for rationally-designing biomolecules as will be described in more detail herein. FIGS. 1-10 show various structures and results related to Example 1. FIGS. 11-14 show various structures and results related to Example 2. FIGS. 15-24 show various structures and results related to Example 3.

### Example 1

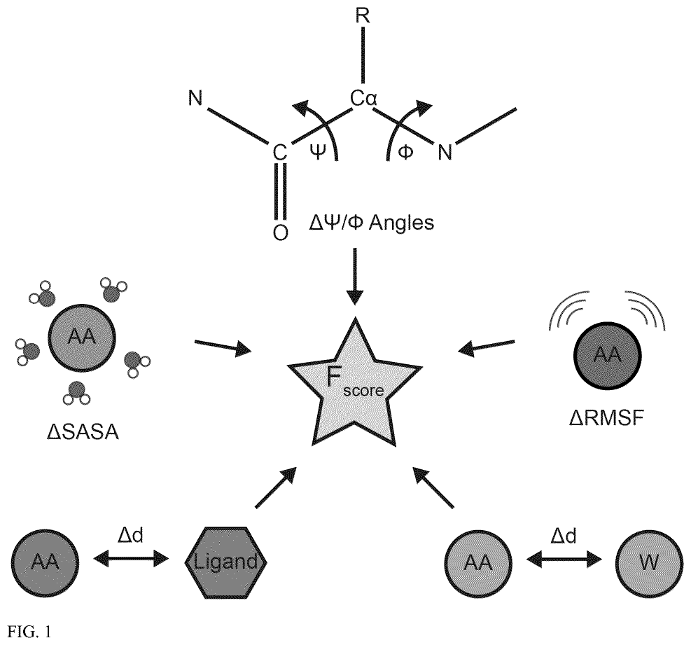

FIG. 1 is a graphical abstract showing an overview of factors that contribute to the F_{score} of an amino acid (AA) residue at a particular position in a ligand-binding polypeptide. ΔSASA refers to solvent accessibility of the amino acid residue; ΔRMSF refers to root mean square fluctuation of the alpha carbon of the amino acid residue; Δd refers to distance between the amino acid residue and the ligand or between the amino acid residue and a tryptophan residue (W).

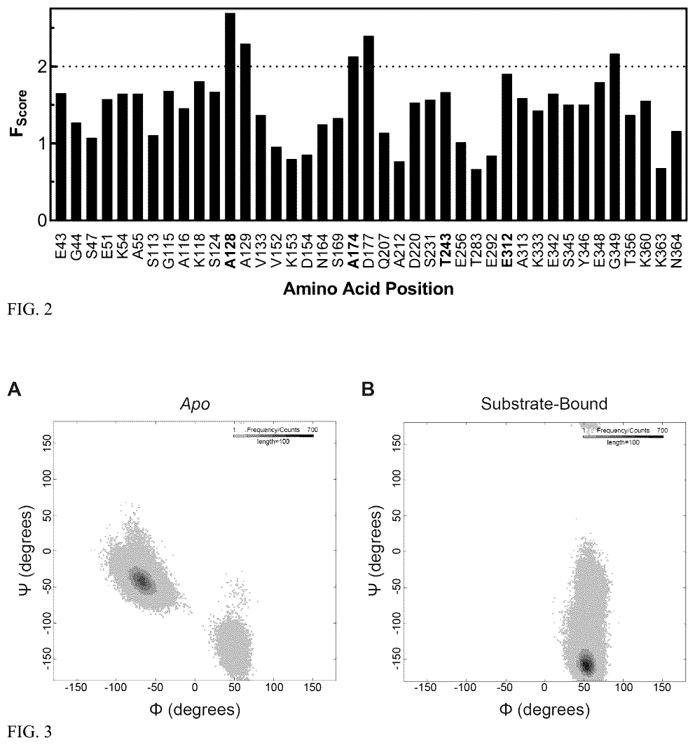

FIG. 2 is an example output of F_{score} values for Streptococcus pneumoniae MalX determined from 100 ns molecular dynamics simulations.

FIG. 3 is a Ramachandran plot for position A128 (alanine-128) from 100 ns apo (A) or ligand-bound (B) molecular dynamics simulations of Streptococcus pneumoniae MalX.

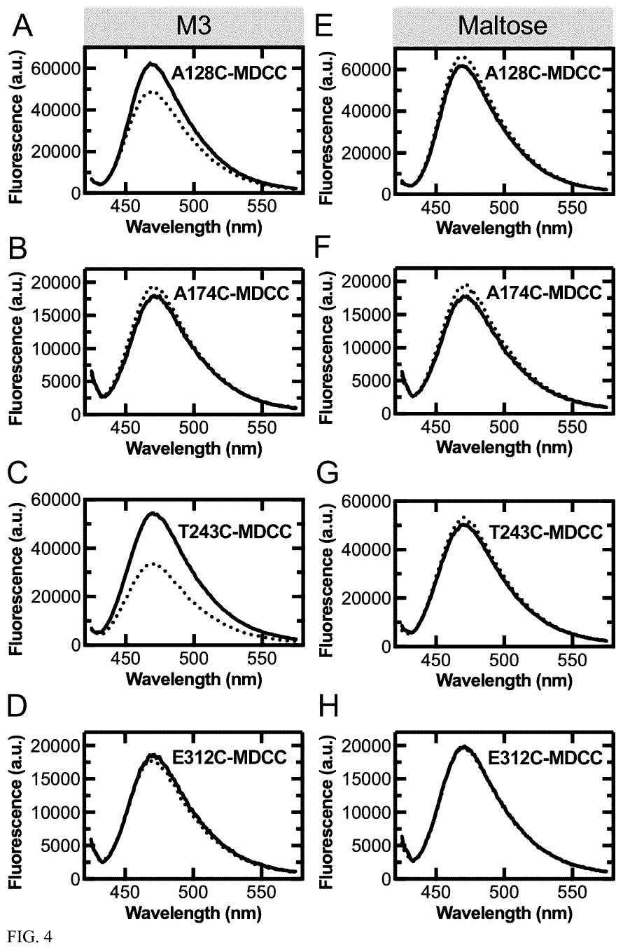

FIG. 4 is a series of charts shoring fluorescence spectra of MOS-detecting biosensors. MOS-detecting biosensor (1 μM) fluorescence in the absence (black solid line) and presence (red dashed line) of 20 μM maltotriose (M3) for Streptococcus pneumoniae MalX variants (A) A128C-MDCC, (C) A174C-MDCC, (E) T243C-MDCC, and (G) E312C-MDCC. MOS-detecting biosensor in the absence (black solid line) and presence (blue dashed line) of 20 μM maltose for Streptococcus pneumoniae MalX variants (B) A128C-MDCC, (D) A174C-MDCC, (F) T243C-MDCC, and (H) E312C-MDCC.

FIG. 5 is a series of charts showing concentration dependence of M3-binding induced fluorescence change in MOS-detecting biosensors. Streptococcus pneumoniae MalX variants (20 nM) bind M3 with an affinity of (A) A128C-MDCC, 190±50 nM and (B) T243C-MDCC, 600±200 nM (n=13 data points, best fit ±95% c.i.) demonstrating the high sensitivity of the biosensor.

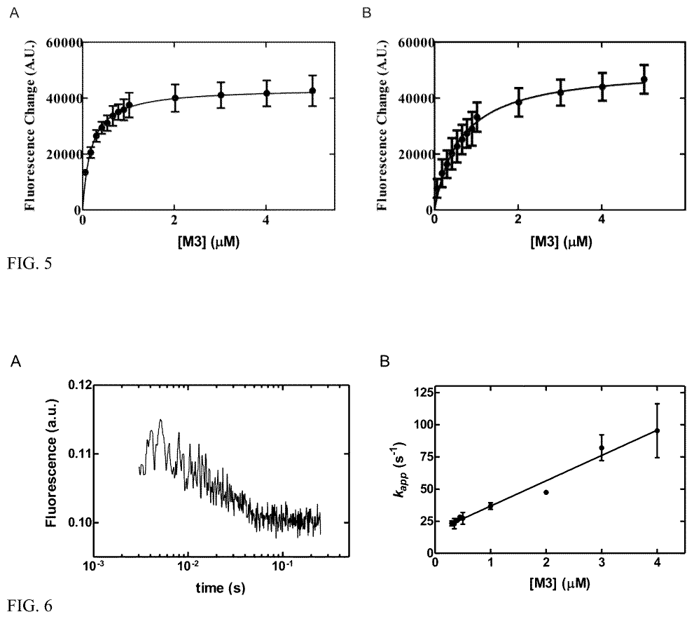

FIG. 6 is a series of charts showing concentration dependence of the M3-association rate. (A) Representative fluorescence time-course of 2 μM M3 binding 100 nM Streptococcus pneumoniae MalX variant A128C-MDCC. (B) Streptococcus pneumoniae MalX variant A128C-MDCC (100 nM) fluorescence signal change across a variety of M3 concentrations (n=4 for each data point, mean±s.d.). Fitting a linear function to the data reports a rate constant of 20±2 μM⁻¹s⁻¹ (n=9, best fit ±s.d.).

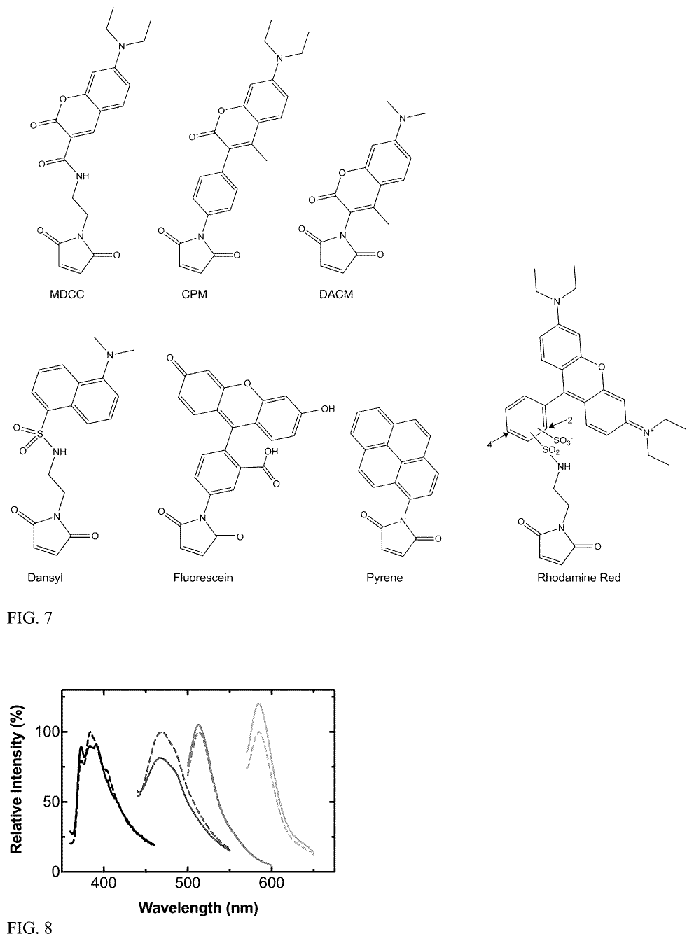

FIG. 7 is a series of structural formulas of thiol-reactive fluorophores (generated via ChemDraw Prime 16.0).

FIG. 8 is a chart showing that Streptococcus pneumoniae MalX variant A128C can be conjugated to a variety of fluorophores to alter fluorescence properties. Fluorescence emission scans of Streptococcus pneumoniae MalX variant A128C conjugated to Pyrene (black), MDCC (blue), Fluorescein (green) or Rhodamine Red (red). Dashed spectra indicate MOS-detecting biosensor alone (20 nM), and solid spectra indicate MOS-detecting biosensor in the presence of MOS (10 μM).

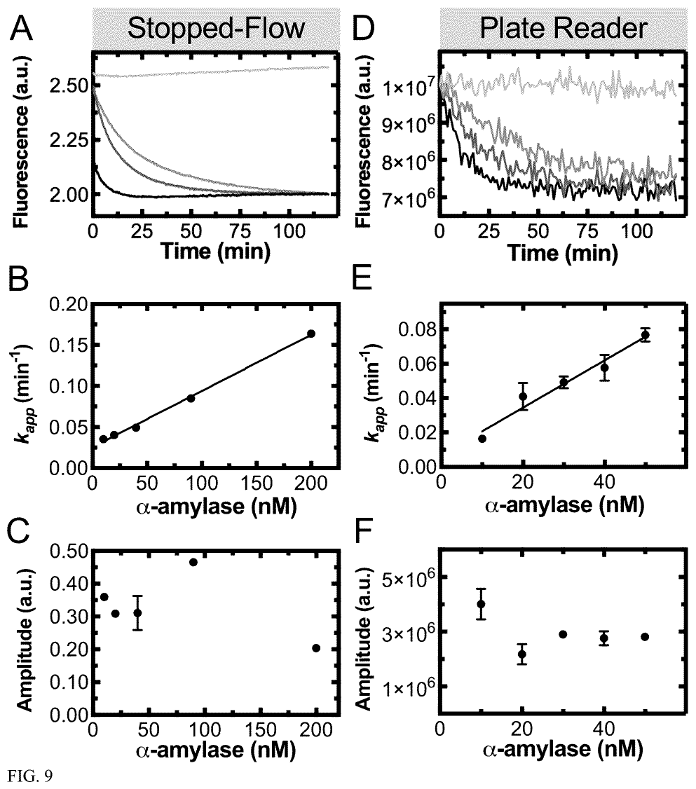

FIG. 9 is a series of charts showing MOS-release from α-amylase-catalyzed starch 10 degradation assayed by Streptococcus pneumoniae MalX variant A128C-MDCC. Representative fluorescence-time courses of Streptococcus pneumoniae MalX variant A128C-MDCC (1.5 μM) in the presence of 100 mg/L starch and (A) α-amylase (0 nM, grey; 40 nM, red; 90 nM, green; 200 nM, blue) using the stopped-flow method, or (B) α-amylase (0 nM, grey; 10 nM, red; 30 nM, green; 50 nM, blue) using a microplate reader. Concentration dependence of α-amylase on apparent rate (kₐₚₚ) (C) in a stopped-flow assay, rate constant of 0.7±0.1 μM⁻¹min⁻¹ or (D) in a microplate reader assay, rate constant of 1.4±0.5 μM⁻¹min⁻¹ (error reflects 95% c.i. in fits). Amplitudes of Streptococcus pneumoniae MalX signal change remain constant as a function of α-amylase concentration (E) using the stopped-flow method or (F) using microplate reader assays (n=3 for each data point).

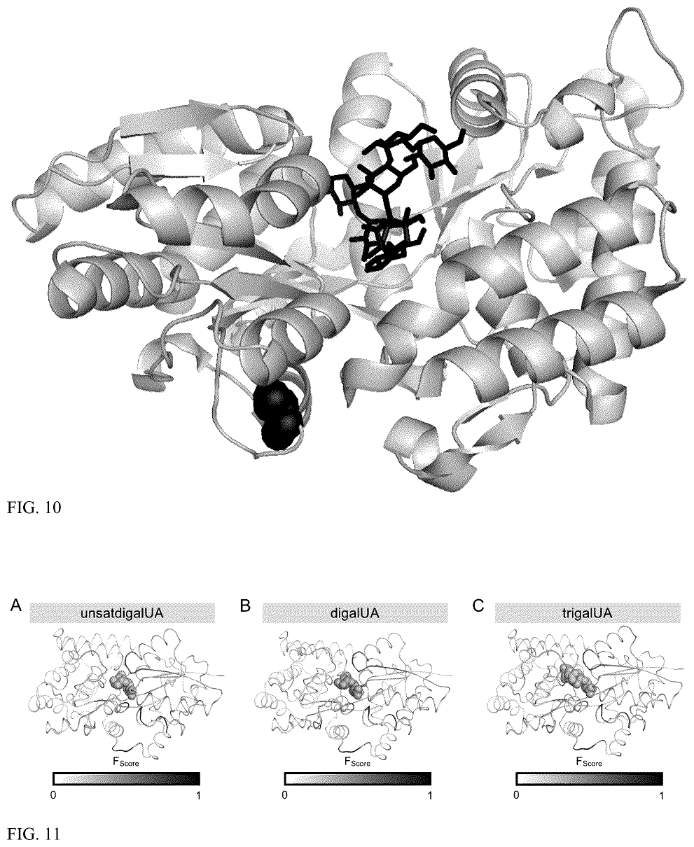

FIG. 10 is a diagram showing that Streptococcus pneumoniae MalX variant A128C-MDCC utilizes a fluorophore conjugation site distal from the MOS-binding pocket. Position A128 (red) is distal from the bound MOS (green) in Streptococcus pneumoniae MalX (cyan, PDB no. 2XD3, figure generated using PyMOL).

### Example 2

FIG. 11 is a series of diagrams showing small-scale changes in protein dynamics upon substrate binding evident using F_{score}. Average F_{score} values for each apo vs. ligand-bound state are projected onto their corresponding Protein Data Bank archive (PDB) structures: Yersinia enterocolitica TogB bound to 4,5-unsaturated digalacturonic acid (unsatdigalUA) (A, PDB 2UVI), Yersinia enterocolitica TogB bound to digalacturonic acid (digalUA) (B, PDB 2UVH), and Yersinia enterocolitica TogB bound to trigalacturonic acid (trigalUA) (C, PDB 2UVJ). Ligand is shown using grey spheres, and protein backbone is shown as ribbon coloured according to F_{score}.

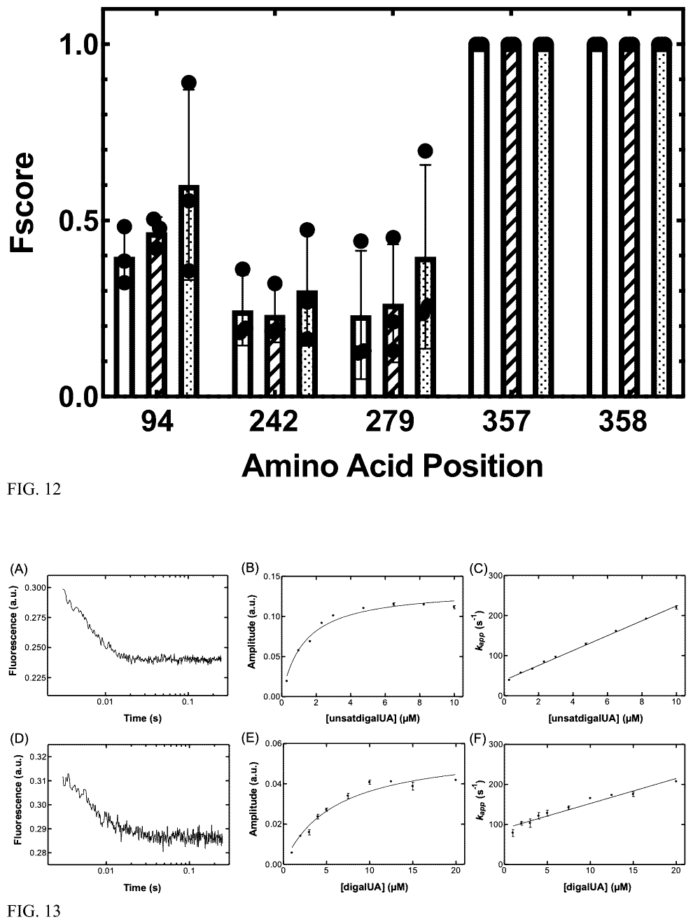

FIG. 12 is a chart showing F_{score} values reflecting changes in dynamics of backbone dihedral angles at candidate labeling positions. Changes in dynamics of candidate labeling positions relative to dynamics in the Yersinia enterocolitica TogB apo state are shown for TogB·unsatdigalUA (white bars), TogB·digalUA (cyan striped bars), and TogB·trigalUA (red dotted bars). Each bar reflects the results of 3 replicates ±s.d. with results from individual trials superimposed on the plot (black dots).

FIG. 13 is a series of charts showing Concentration dependence of unsatdigalUA-association rate and digalUA-association rate. Representative fluorescence time-course of 100 nM Yersinia enterocolitica TogB variant D358C-MDCC binding 10 μM unsatdigalUA (A) or 10 μM digalUA (D). Fluorescence time-courses were obtained for 100 nM Yersinia enterocolitica TogB variant D358C-MDCC binding to substrate across a range of carbohydrate concentrations (0.3-10 μM for unsatdigalUA, and 1-20 μM for digalUA). Fluorescent-time courses were fit with a one exponential function (Equation 18) to determine Amplitude and kₐₚₚ. Amplitudes of signal change were plotted against concentrations of unsatdigalUA (B) and digalUA (E) and fit with a hyperbolic function (Equation 20) to determine dissociation constant (K_{D}=1.3±0.5 μM for unsatdigalUA, K_{D}=6±1 μM for digalUA). kₐₚₚ was plotted against concentrations of unsatdigalUA (C) and digalUA (F) and fit with a linear function to determine association constants (kₒₙ=18.6±0.7 μM⁻¹s⁻¹ for unsatdigalUA, and 6±1 μM⁻¹s⁻¹ for digalUA).

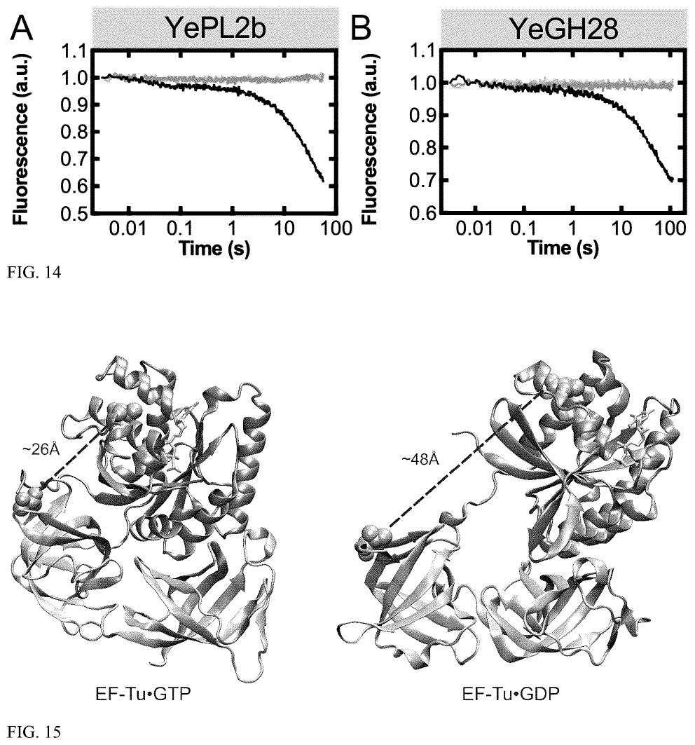

FIG. 14 is a series of charts showing oligogalacturonide-release from CAZyme-catalyzed degradation of polygalacturonic acid (PGA) detected by Yersinia enterocolitica TogB variant D358C-MDCC. Representative fluorescence time-courses for product released by 250 nM YePL2b (A, Black fluorescence time-course) or 250 nM YeGH28 (B, Black fluorescence time-course) in the presence of 0.5 mg/L PGA detected by 250 nM Yersinia enterocolitica TogB variant D358C-MDCC. Negative controls in the absence of CAZyme (Red fluorescence time-courses) and in the absence of PGA (Blue fluorescence time-courses) are shown.

### Example 3

FIG. 15 is a series of models showing the conformational differences between Escherichia coli EF-Tu bound to GTP or GDP. The three domains of EF-Tu are shown, domain 1 or the G-domain (blue), domain 2 (red), and domain 3 (green). Structures of EF-Tu bound to GDP and GTP were generated using the Visual Molecular Dynamics software (VMD) using PDB ID 1EFC and PDB ID 1TTT, respectively. Amino acid positions 34 and 265 are shown as purple spheres.

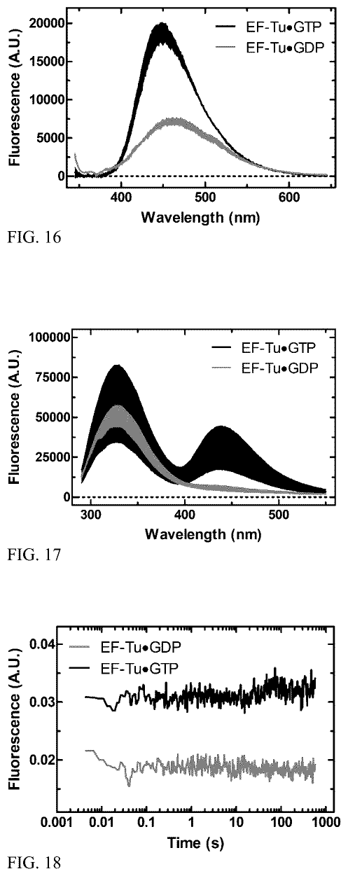

FIG. 16 is a chart showing fluorescence emission spectra of double labelled Escherichia coli EF-Tu T34C L265C-IAEDANS/DDPM. Using a fluorescence spectrophotometer (PTI), double labelled EF-Tu was excited at 336 nm and an emission spectrum was recorded between 345-650 nm. Top and bottom lines of each trace represent one standard deviation.

FIG. 17 is a chart showing fluorescence emission spectra of single labelled Escherichia coli EF-Tu L265C-Dansyl. Using a fluorescence spectrophotometer (PTI), labelled EF-Tu was excited at 280 nm and an emission spectrum was recorded between 290-550 nm. Top and bottom lines of each trace represent one standard deviation.

FIG. 18 is a chart showing fluorescence change of single labelled Escherichia coli EF-TuL265C-Dansyl upon mixing with 10 mM EDTA. Labelled EF-Tu was excited at 280 nm and an emission was recorded using a high pass 350 nm filter.

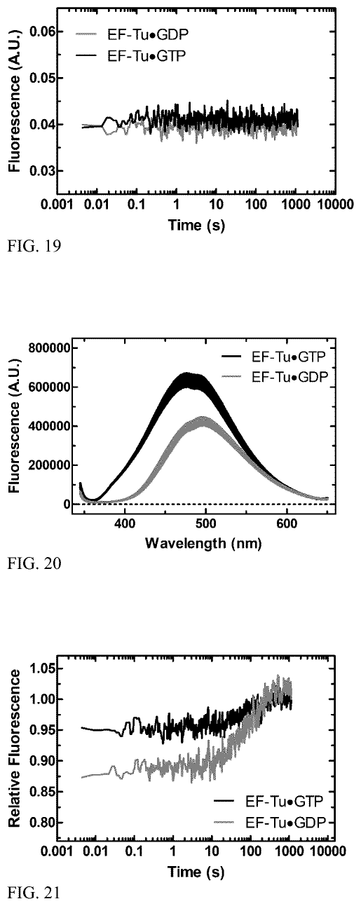

FIG. 19 is a chart showing fluorescence change of single labelled Escherichia coli EF-Tu L265C-Dansyl upon mixing with 10 mM EDTA. Labelled EF-Tu was excited at 335 nm and an emission was recorded using a high pass 350 nm filter.

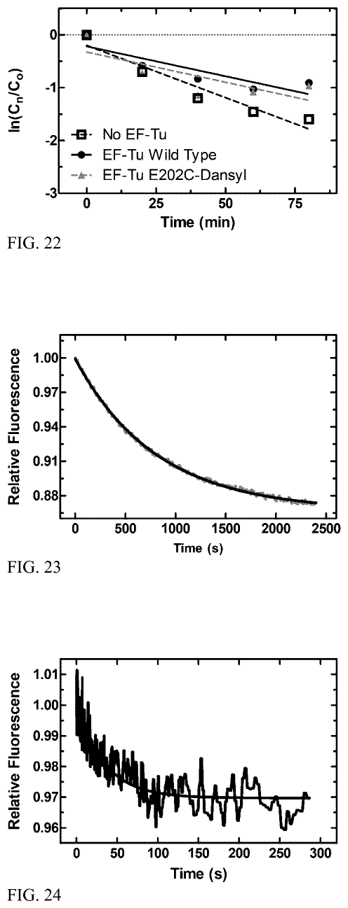

FIG. 20 is a chart showing fluorescence emission spectra of single labelled Escherichia coli EF-Tu E202C-Dansyl. Using a fluorescence spectrophotometer (PTI), labelled EF-Tu was excited at 335 nm and an emission spectrum was recorded between 345-650 nm. Top and bottom lines of each trace represent one standard deviation.

FIG. 21 is a chart showing fluorescence change of single labelled Escherichia coli EF-TuE202C-Dansyl upon mixing with 10 mM EDTA. Labelled EF-Tu was excited at 335 nm and an emission was recorded using a high pass 350 nm filter.

FIG. 22. is a chart showing Escherichia coli EF-Tu E202C-Dansyl·GTP·aa-tRNA ternary complex stability. The time dependence of [14C]Phe-tRNA_{Phe} hydrolysis, incubated in the presence of EF-Tu (Ln(Cₙ/Cₒ)) is plotted against time, where the slope is the rate of aminoacyl-ester bond cleavage. Cₙ is the concentration of tRNA_{Phe} at a given time point and Cₒ is the concentration of tRNA_{Phe} at time 0.

FIG. 23 is a chart showing fluorescence change upon mantGDP dissociation from single labelled Escherichia coli EF-Tu E202C-Dansyl. Measured via FRET excitation of mant fluorescence where tryptophan was excited at 280 nm and an emission was recorded using a long-pass 400 nm filter.

FIG. 24 is a chart showing fluorescence change upon mantGTP dissociation from single labelled Escherichia coli EF-Tu E202C-Dansyl. Measured via FRET excitation of mant fluorescence where tryptophan was excited at 280 nm and an emission was recorded using a long-pass 400 nm filter.

### DETAILED DESCRIPTION OF INVENTION

According to embodiments, improved processes for rationally-designing and producing biomolecules are disclosed. More specifically, embodiments herein are directed to non-stochastic processes for selecting target positions within a biomolecule that are suitable for modification. In some embodiments, the present processes may comprise the steps of (i) selecting at least one biomolecule suitable for modification, (ii) obtaining at least one structure of the at least one biomolecule, (iii) simulating the molecular dynamics of the at least one structure to generate dynamic information about at least one position within the at least one structure, (iv) using the dynamic information to calculate a score for the at least one position, (v) comparing the score with at least one reference score to identify at least one target position within the biomolecule suitable for modification, and (vi) modifying the at least one target position in order to produce a rationally-designed biomolecule.

By way of example, present processes may be used to rationally design and produce MOS-detecting biosensors based on the Streptococcus pneumoniae MalX biomolecule (Example 1; SEQ ID NO: 1). Present processes may also be used to rationally design and produce HPB-detecting biosensors based on the Yersinia enterocolitica TogB biomolecule (Example 2; SEQ ID NO: 2). Present processes may further be used to rationally design and produce biosensors for observing conformational changes based on the Escherichia coli EF-Tu biomolecule (Example 3; SEQ ID NO: 3).

### Step 1: Selection of Biomolecules Suitable for Modification

According to embodiments, the present processes for designing and producing biomolecules may comprise a step of selecting at least one biomolecule suitable for modification. In some embodiments, suitable biomolecules may include compounds that are naturally-occurring in organisms and have a function related to biological processes including cell division, morphogenesis, and development. It is understood, however, that biomolecules are not limited to compounds that are found in nature and may also include stereoisomers of naturally-occuring biomolecules and other synthetic or engineered biomolecules that do not occur naturally. In other embodiments, suitable biomolecules may include polypeptides, nucleic acids, lipids, carbohydrates, and any combination thereof, whether comprising a single homogenous compound or a complex formed of homogenous or heterogenous subunits. In yet other embodiments, suitable biomolecules may be characterized by a particular structure that determines its function.

Suitable biomolecules are those that may be readily selected based on one or more criteria. In some embodiments, such criteria may comprise whether the biomolecule binds a ligand of interest and, further, whether the biomolecule has a suitable specificity and affinity for the ligand. In other embodiments, such criteria may comprise whether the biomolecule is well-understood and, further, whether high-resolution three-dimensional atomic structures of the biomolecule are known. In yet other embodiments, such criteria may comprise whether the biomolecule is naturally-expressed in a convenient expression system. In yet other embodiments, such criteria may comprise the size of the biomolecule, where smaller biomolecules may be preferable to simulate in molecular dynamic simulations. In yet other embodiments, such criteria may comprise whether the biomolecule is known to have advantageous secondary functions. The examples listed above are not exhaustive. It is appreciated that the relevant criteria, as well as their respective weighting, depends on the desired functionality of the resultant biomolecule variant and the types of modifications that are suitable to achieve that desired function.

As will be described in more detail, the desired functionality of the resultant biomolecule variant and the types of modifications that are suitable to achieve such desired functionality may be categorized according to four types of structural modifications: addition of reporter groups, addition of linkers, intramolecular modifications, and addition of compounds for medical treatment. Notwithstanding anything contained herein, it is generally appreciated what types of structural modifications may be made to produce a variant with a desired function.

### Step 1a: Selection of Biomolecules Suitable for Modification by Addition of a Reporter Group

A biomolecule's structure may be modified by the addition of a reporter group to confer biosensing functionality on the resultant biomolecule variant. For example, a variety of reporter groups can be used, differing in the physical nature of signal transduction (e.g., fluorescence, electrochemical, nuclear magnetic resonance (NMR), and electron paramagnetic resonance (EPR)) and in the chemical nature of the reporter group. Useful reporter groups include, but are not limited to, fluorophores and redox cofactors. These reporter groups tend to generate a signal change that corresponds with changes in their local environment including relative orientation in three-dimensional space, pH, temperature, and ionic strength.

In the case of fluorophores, the selection of a particular fluorophore may depend upon, at least in part, the nature of the target position within the biomolecule. For example, in circumstances where one fluorophore may generate a larger signal change at a particular target position compared to another, a preferred fluorophore may be selected for a particular application (see, for example, U.S. Pat. No. 6,277,627). In the first example that follows, seven different fluorophores are used in the design and production of MOS-detecting biosensors that generate suitable signal changes. In the second example that follows, only one fluorophore was used in the design and production of HBP-detecting biosensors that generate suitable signal changes. In the third example that follows, three different fluorophores are used in the design and production of biosensors for observing conformational changes within a biomolecule that generate suitable signal changes, with two of the different fluorophores operating as a Forster resonance energy transfer (FRET) pair within the same biomolecule. The present processes, however, are in no way limited to these specific embodiments.

As used herein, the term “fluorophore” relates to a functional group in a compound which will absorb energy of a specific wavelength and re-emit energy at a different (but equally specific) wavelength. In some embodiments, fluorophores may exhibit intramolecular spectral properties or intermolecular spectral properties when energetically-linked to other compounds, functional groups, or fluorophores through phenomena such as FRET. In some embodiments, fluorophores may be small molecules including 7-diethylamino-3-(4′-maleimidylphenyl)-4-methylcoumarin (CPM), 7-diethylamino-3-[N-(2-maleimidoethyl)carbamoyl]coumarin (MDCC), N-(7-dimethylamino-4-methylcoumarin-3-yl)maleimide (DACM), N-[2-(dansylamino)ethyl]maleimide (Dansyl), fluorescein-5-maleimide (Fluorescein), N-(1-pyrene)maleimide (Pyrene), Rhodamine Red C2 maleimide (Rhodamine Red), and 5-(2-iodoacetylaminoethyl)aminonaphthalene-1-sulfonic acid (IAEDANS). Such fluorophores may be members of the naphthalene family, xanthene family, and pyrene family of small molecules. In other embodiments, fluorophores may be macromolecules including polypeptides. For example, the fluorophore in green fluorescent protein (GFP) includes Ser-Tyr-Gly sequence (i.e., Ser65-dehydroTyr66-Gly67), which is post-translationally modified to a 4-(p-hydroxyben-zylidene)-imidazolidin-5. Exemplary genetically encoded fluorescent proteins include, but are not limited to, fluorescent proteins from coelenterate marine organisms, e.g., Aequorea victoria, Trachyphyllia geoffroyi, coral of the Discosoma genus, Rennilla mulleri, Anemonia sulcata, Heteractis crispa, Entacmaea quadricolor, and/or GFP (including the variants S65T and EGFP, Rennilla mulleri GFP), cyan fluorescent protein (CFP), including Cerulean, and mCerulean3 (described by MARKWARDT et al., PLoS ONE, 6(3) el 7896.doi:10.1371/journal.pone.0017896), CGFP (CFP with Thr203Tyr: Has an excitation and emission wavelength that is intermediate between CFP and EGFP), yellow fluorescent protein (YFP, e.g., GFP-Ser65Gly/Ser72Ala/Thr203Tyr; YFP (e.g., GFP-Ser65Gly/Ser72Ala/Thr203Tyr) with Val68Leu/Gln69Lys); Citrine (i.e., YFP-Val68Leu/Gln69Met), Venus (i.e., YFP-Phe46Leu/Phe64Leu/Met153ThrNal163Ala/Ser175Gly), PA-GFP (i.e., GFP-Val/163Ala/Thr203His), Kaede), red fluorescent protein (RFP, e.g., long wavelength fluorescent protein, e.g., DsRed (DsRed1, DsRed2, DsRed-Express, mRFPl, drFP583, dsFP593, asFP595), eqFP611, and/or other fluorescent proteins known in the art (see, e.g., ZHANG et al., Nature Reviews, Molecular and Cellular Biology, 2002, 3:906-908). In other embodiments, fluorophore containing molecules include fluorescent proteins that can be or that are circularly permutated. Circular permutation methods are known in the art (see, e.g., BAIRD et al., Proc. Natl. Acad. Sci., 1999, 96:11241-11246; TOPELL, GLOCKSHUBER, Methods in Molecular Biology, 2002, 183:31-48). In other embodiments, fluorophores can include circularly permuted YFP (cpYFP) as a circularly permutated fluorescent protein (cpFP). cpYFP has been used as a reporter element in the creation of biosensors for H₂O₂ (HyPer) (BELOUSOV et al., Nat. Methods, 2006, 3:281-286), cGMP (FlincG) (NAUSCH et al., Proc. Natl. Acad. Sci. USA., 2008, 105: 365-370), ATP:ADP ratio (Perceval) (BERG et al., Nat. Methods., 2008, 105:365-370), and calcium ions (NAKAI et al., Nat. Biotechno., 2001, 19:137-141), including full length, fragments, and/or variants thereof.

Redox cofactors may also be readily selected for a particular application (see, for example, U.S. Pat. No. 6,277,627). Such redox-active reporter groups are attached to the biomolecule so that they are located between the biomolecule and an electrode. Redox cofactors can be a redox-active metal center or a redox-active organic molecule. Redox cofactors can be a natural organic cofactor such as nicotinamide adenine dinucleotide (NAD), nicotinamide adenine dinucleotide phosphate (NADP), or flavin adenine dinucleotide (FAD), or a natural metal center such as Blue Copper, iron-sulfur clusters, or heme, or a synthetic center such as an organometallic compound such as a ruthenium complex, organic ligand such as a quinone, or an engineered metal center introduced into the biomolecule or engineered organic cofactor binding site. Cofactor-binding sites can be engineered using rational design or directed evolution techniques. Redox cofactors may be covalently or non-covalently attached to the biomolecule, either by site-specific or adventitious interactions between the cofactor and biomolecule. Redox cofactors may be intrinsic to the biomolecule such as a metal center (natural or engineered) or natural organic (NAD, NADP, FAD) or organometallic cofactor (heme), or extrinsic (such as a covalently conjugated, synthetic organometallic cluster). Redox cofactors may be, for example, bound (e.g., covalently) at a position on the biomolecule's surface (e.g. solvent-accessible positions).

In some embodiments, redox cofactors can be a metal-containing group (e.g., a transition metal-containing group) capable of reversibly or semi-reversibly transferring one or more electrons. A number of possible transition metal-containing redox cofactors can be used. Advantageously, the redox cofactor may comprise a redox potential in the potential window below that which is subject to interference by molecular oxygen and has a functional group suitable for covalent conjugation to the biomolecule (e.g., thiol-reactive functionalities such as maleimides or iodoacetamide for coupling to unique cysteine residues in a polypeptide). The metal of the redox cofactor should be substitutionally inert in either reduced or oxidized state (i.e., advantageously, exogenous groups do not form adventitious bonds with the redox-active reporter group). The redox-active reporter group can be capable of undergoing an amperometric or potentiometric change in response to ligand binding. In a preferred embodiment, the reporter group may be water soluble, capable of site-specific coupling to a biomolecule (e.g., via a thiol-reactive functional group on the reporter group that reacts with a unique cysteine in a polypeptide), and undergo a potentiometric response upon ligand binding. Suitable transition metals for use in the invention include, but are not limited to, copper (Cu), cobalt (Co), palladium (Pd), iron (Fe), ruthenium (Ru), rhodium (Rh), osmium (Os), rhenium (Re), platinum (Pt), scandium (Sc), titanium (Ti), vanadium (V), chromium (Cr), manganese (Mn), nickel (Ni), molybdenum (Mo), technetium (Tc), tungsten (W), and iridium (Ir). That is, the first series of transition metals, the platinum metals (Ru, Rh, Pd, Os, Ir, and Pt), along with Fe, Re, W, Mo, and Tc, are preferred. Particularly preferred, transition metals may be metals that do not change the number of coordination sites upon a change in oxidation state, including, without limitation, ruthenium, osmium, iron, platinum and palladium, with ruthenium being especially preferred.

In some embodiments, the biomolecule may be modified (i.e. “labeled”) by a suitable reporter group at a target position within the biomolecule. Labelling may comprise attaching the reporter group to the target position covalently or non-covalently. Such attachment may comprise direct interaction between the reporter group and the target position. For instance, the reporter group can be present as a covalent conjugate with the target position or it can be a metal center that forms part of the biomolecule matrix (for instance, a redox center such as iron-sulfur clusters, heme, Blue copper, the electrochemical properties of which are sensitive to its local environment). Alternatively, such attachment may comprise indirect interaction between the reporter group and the target position. Such indirect attachment means may comprise further modifications to enable the reporter group to be attached to the target position including the addition of a linker inserted between the reporter group and the target position or an intramolecular mutation of the target position that enables the reporter group to be attached to the target position. For example, in the case of polypeptides, such linkers or intramolecular modifications can include at least one naturally occurring or synthetic amino acid and, in some embodiments, the reporter group may be covalently conjugated to the polypeptide via a maleimide functional group bound to a cysteine (thiol) on the polypeptide. Irrespective of attachment means, the reporter group can also be present in the biomolecule as a fusion between the biomolecule and a metal binding domain (for instance, a small redox-active protein such as a cytochrome).

In some embodiments, attaching these reporter groups to a biomolecule may exploit the specificity and affinity that some biomolecules have for their substrates. As the biomolecule binds to an analyte, global and local conformational changes within the biomolecule may alter the local environment of the reporter group and, thus, generate a signal change. In other embodiments, the detectable signal is detectably distinct (e.g., can be distinguished using methods known in the art and/or disclosed herein) from a signal emitted by the molecule prior to inducement (e.g., reporter groups can emit a signal in at least two detectably distinct states: for example, a first signal can be emitted in an apo state and a second signal can be emitted in a ligand-bound state). Furthermore, such biomolecules may have a transient or cryptic structure (e.g. a structure that only exists between states) that is detectably distinct from either apo or ligand-bound states. In some instances, the conformational change that occurs upon interaction with an analyte (e.g., an analyte-binding dependent conformational alteration) is detectably distinct (e.g., can be observed using methods known in the art) from a conformational change that may occur for the same biomolecule under other physiological conditions (e.g., a change in conformation induced by altered temperature, pH, voltage, ion concentration, phosphorylation). In yet other embodiments, the detectable signal is proportional to the degree of inducement. In yet other embodiments, if two or more reporter groups are attached to two or more target positions within a biomolecule, then two or more detectably distinct signals may be emitted by the biosensor. Such configurations may be desirable for reporter groups where issues arising from long-term effects such as degradation may arise. For example, such issues can be identified by fusing an intensity-based biosensor to another reporter group with a detectably distinct signal, to serve as a reference channel.

Regardless of reporter group configuration, methods of selecting a biomolecule suitable for modification by the one or more reporter groups are known and generally comprise using criteria including those identified above or elsewhere herein. For example, methods for identifying suitable biomolecules that exhibit suitable conformational characteristics and/or for observing differences in structure between structures or before and after a conformational change are known, including, for example, one or more of structural analysis, crystallography, NMR, EPR using Spin label techniques, Circular Dichroism (CD), Hydrogen Exchange surface Plasmon resonance, calorimetry, and/or FRET. According to embodiments, however, suitable conformation characteristics need not be known prior to selection of the presently claimed biomolecule, provided that at least one static structure of the biomolecule is known or has been modeled. Other criteria and corresponding selection methods are known, as discussed in more detail above or elsewhere herein.

In some instances, suitable biomolecules may interact specifically with one analyte (e.g., at least one defined, specific, and/or selected analyte). In such cases, affinity of binding between the biomolecule and the analyte can be high or can be controlled (e.g., with millimolar, micromolar, nanomolar, or picomolar affinity). Alternatively, single biomolecules may bind two or more analytes (e.g., two or more defined, specific, and/or selected analytes). In such cases, affinity of binding to the two or more analytes can be the same or distinct. For example, the affinity of binding can be greater for one analyte than it is for a second or third, etc., analyte. In some instances, affinity of binding between the suitable biomolecule and an analyte (e.g., at least one defined, specific, and/or selected analyte) may be within the range of 1 μM to 10 mM.

In some embodiments, one or more biomolecules may be suitable, wherein the one or more biomolecules each bind (e.g., bind specifically) a single analyte (e.g., a single defined, specific, and/or selected analyte) or distinct analytes (e.g., two or more distinct defined, specific and/or selected analytes). In some embodiments, the one or more biomolecules can be chimeric. In such embodiments, a first part of the biomolecule can be a first biomolecule subunit or can be derived from a first biomolecule subunit, and a second part of the biomolecule can be a second biomolecule subunit or can be derived from a second biomolecule subunit, wherein the first and second biomolecule subunits are combined to result in the at least one or more biomolecules (e.g. a chimeric biomolecule).

In some instances, the suitable biomolecules can be a bacterial polypeptide or can be derived from a bacterial polypeptide. Suitable bacterial polypeptides can include, but are not limited to, for example, Streptococcus pneumoniae MalX, Yersinia enterocolitica TogB, and Escherichia coli EF-Tu. As will be shown, MOS-detecting biosensors may be based on the Streptococcus pneumoniae MalX biomolecule for at least the reason that it exhibits a high affinity and specificity for MOS analytes having a degree of polymerization of between three to eleven glucose residues. As will also be shown, HBP-detecting biosensors may be based on the Yersinia enterocolitica TogB biomolecule for at least the reason that it exhibits variable affinity and specificity for at least three HBP analytes including 4,5-unsaturated digalacturonic acid, digalacturonic acid, and trigalacturonic acid. As will also be shown, biosensors for observing conformational changes within a biomolecule may be based on the Escherichia coli EF-Tu protein for at least the reason that it is predicted to exhibit conformational changes.

Although certain embodiments have been described, such embodiments are in no way intended to limit the rational design and production processes disclosed herein. It is contemplated that the presently described design and production processes may be used with any number of biomolecules, provided that a structure of the biomolecule is known or modelled and can be subjected to molecular dynamics simulations.

Furthermore, it should be understood that any of the biomolecules, reporter groups, or resultant biomolecule variants described herein can be modified and varied, provided that their desired function may be maintained. For example, it is contemplated that the Streptococcus pneumoniae MalX, Yersinia enterocolitica TogB, and Escherichia coli EF-Tu biosensors disclosed herein could be modified as long as the resulting variants have the same or better characteristics as the biomolecule from which they derived, with, for example, such variants having the same or better affinity for their respective ligands (where better affinity refers to greater or lesser affinity, whichever may be more desirable in a particular circumstance). As a further example, such same or better characteristics may comprise maintaining the ligand-interacting face or ligand binding pocket within a variant (e.g., substantially the same) as compared to the biomolecule from which the variant is derived (methods for identifying the interacting face or ligand binding pocket of a biomolecule are known in the art (Gong et al., BMC: Bioinformatics, 6:1471-2105 (2007); Andrade and Wei et al., Pure and Appl. Chem., 64(11):1777-1781 (1992); Choi et al., Proteins: Structure, Function, and Bioinformatics, 77(1):14-25 (2009); Park et al., BMC: and Bioinformatics, 10:1471-2105 (2009)), e.g., to maintain binding to a ligand. Alternatively, residues or subunits (e.g. amino acids, nucleotides, sugars, etc.) within the ligand binding pocket or interacting face can be modified, e.g., to decrease binding to a ligand and/or to change ligand specificity. The ligand binding pocket or interacting face of a biomolecule is the region of the biomolecule that interacts or associates with a ligand. Generally, residues or subunits within the ligand binding pocket or interacting face are naturally more highly conserved than those located elsewhere. In some embodiments, for example, an amino acid within the ligand binding pocket or interacting face region of any polypeptide or variant thereof can be the same as the amino acid shown in any of the polypeptides or variants thereof, or can include conservative amino acid substitutions. In some embodiments, for example, an amino acid within the ligand binding pocket or interacting face region of any polypeptide or variant thereof can be substituted with an amino acid that increases the interaction between the polypeptides or polypeptide variants and a ligand. In some embodiments, a genetically encoded polypeptide variant may comprise polypeptides having at least 80, 85, 90, 95, 96, 97, 98, 99 percent identity to the polypeptide, reporter group, or polypeptide variant, such identify of the two polypeptides being readily identifiable. For example, such polypeptide identity can be calculated by aligning the two sequences to achieve the identity at its highest level. Other methods of calculating identity may comprise using known identity alignment algorithms. Such known algorithms may also be used to calculate identity of nucleic acids, which may have similar conservation characteristics as polypeptides. It is understood that any of the methods typically can be used and that in certain instances the results of these various methods may differ, but if identity is found with at least one of these methods, the sequences would be said to have the stated identity and to be disclosed herein.

In some embodiments, one or more intramolecular modifications may be made to the suitable biomolecules. Such intramolecular modifications typically fall into one or more of three classes: substitutional, insertional, or deletional modifications. In the case of polypeptides, for example, insertions include amino and/or terminal fusions as well as intra-sequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the polypeptide sequence. Typically, no more than about from 2 to 6 residues are deleted at any one site within the protein molecule. Amino acid substitutions are typically of single residues, but can occur at a number of different locations at once; insertions usually will be on the order of about from 1 to 10 amino acid residues; and 5 deletions will range about from 1 to 30 residues. Deletions or insertions can be made in adjacent pairs, i.e., a deletion of residues or insertion of residues. Substitutions, deletions, insertions or any combination thereof may be combined to arrive at a final construct, provided that such changes must not place the sequence out of reading frame, and preferably will not create complementary regions that could produce secondary mRNA structure. Substitutional modifications are those in which at least one residue has been removed and a different residue inserted in its place. In some instances, substitutions can be conservative amino acid substitutions. In some embodiments, suitable polypeptide variants can include one or more conservative amino acid substitutions. For example, such variants can include 1, 2, 3, 4, 5, 6, 7, 8, 9, 10, 11, 12, 13, 14, 15, 16, 17, 18, 19, 20, 20-30, 30-40, or 40-50 conservative amino acid substitutions. Alternatively, variants can include 50 or fewer, 40 or fewer, 30 or fewer, 20 or fewer, 10 or fewer, 9 or fewer, 8 or fewer, 7 or fewer, 6 or fewer, 5 or fewer, 4 or fewer, 3 or fewer, or 2 or fewer conservative amino acid substitutions. Such substitutions generally are made in accordance with the following Table 1 and are referred to as conservative substitutions. Methods for predicting tolerance of conservative substitutions are known.

TABLE 1
Conservative Amino Acid Substitutions

| Amino Acid | | | |
|---|---|---|---|
| | Three-Letter | Single Letter | Substitutions (others |
| Name | Code | Code | are known in the art) |
| Alanine | Ala | A | Ser, Gly, Cys |
| Arginine | Arg | R | Lys, Gln, His |
| Asparagine | Asn | N | Gln, His, Glu, Asp |
| Aspartic Acid | Asp | D | Glu, Asn, Gln |
| Cysteine | Cys | C | Ser, Met, Thr |
| Glutamine | Gln | Q | Asn, Lys, Glu, Asp, Arg |
| Glutamic Acid | Glu | E | Asp, Asn, Gln |
| Glycine | Gly | G | Pro, Ala, Ser |
| Histidine | His | H | Asn, Gln, Lys |
| Isoleucine | Ile | I | Leu, Val, Met, Ala |
| Leucine | Leu | L | Ile, Val, Met, Ala |
| Lysine | Lys | K | Arg, Gln, His |
| Methionine | Met | M | Leu, Ile, Val, Ala, Phe |
| Phenylalanine | Phe | F | Met, Leu, Tyr, Trp, His |
| Serine | Ser | S | Thr, Cys, Ala |
| Threonine | Thr | T | Ser, Val, Ala |
| Tryptophan | Trp | W | Tyr, Phe |
| Tyrosine | Tyr | Y | Trp, Phe, His |
| Valine | Val | V | Ile, Leu, Met, Ala, Thr |

In some instances, substitutions are not conservative. For example, an amino acid can be replaced with an amino acid that can alter some property or aspect of the suitable polypeptide. In some instances, non-conservative amino acid substitutions can be made, e.g., to change the structure of a peptide, to change the binding properties of a peptide (e.g., to increase or decrease the affinity of binding of the peptide to an analyte and/or to alter increase or decrease the binding specificity of the peptide).

The disclosure also features nucleic acids encoding the biosensors described herein, including variants and/or fragments of the biosensors. These sequences include all degenerate sequences related to the specific polypeptide sequence, i.e., all nucleic acids having a sequence that encodes one particular polypeptide sequence as well as all nucleic acids, including degenerate nucleic acids, encoding the disclosed variants and derivatives of the polypeptide sequences. Thus, while each particular nucleic acid sequence may not be written out herein, it is understood that each and every sequence is in fact disclosed and described herein through the disclosed polypeptide sequences.

Furthermore, the ligand-binding pocket may be modified to bind ligands which are not bound by the unmodified biomolecule. In the case of polypeptides, for example, mutating amino acid residues that are near (i.e., in or around) the binding site of a polypeptide may generate new contacts with ligand and destroy or alter binding with cognate ligand. This can be used to change the specificity of the ligand binding pocket.

Other mutations in the suitable biomolecule may be made to affect function of the biomolecule: e.g., mutations may increase or decrease binding affinity or specificity for a ligand; enhance or reduce signal transduction of a reporter group; add a new functionality by fusion with another nucleic acid, carbohydrate, lipid, or polypeptide residue, subunit, or domain; improve thermostability or thermolability of the biomolecule; introduce a catalytic activity to the biomolecule; shorten or lengthen operational life of the biomolecule; widen or narrow the conditions for operation of a biomolecule; or any combination thereof. Preferred is mutating positions of the biomolecule variant in which a reporter group is not attached (e.g., in the case of polypeptides, at least one missense mutation which is not a cysteine conjugated through a thiol bond to a fluorophore).

### Step 1b: Selection of Biomolecules Suitable for Modification by Addition of a Linker

In some embodiments, a biomolecule's structure may be modified by the addition of one or more linkers to confer linker functionality on the resultant biomolecule variant. In such embodiments, the biomolecule may be modified by the linkers at one or more target position within the biomolecule. In other embodiments, linkers may permit the reversable or irreversible interaction between the biomolecule and one or more target ligand. In yet other embodiments, linkers may enable highly-specific covalent or non-covalent conjugation between the linkers and one or more functional group of the biomolecule as well as between the linkers and one or more functional group of the target ligand. In yet other embodiments, linkers may comprise a metal ion or small molecule (including functional groups) that enable the direct binding of the biomolecule to one or more ligands. In yet other embodiments, linkers may comprise a single residue or subunit of a biomolecule (e.g. amino acid, nucleotide, sugar, etc.) that mediate interactions between the biomolecule and one or more ligands. In yet other embodiments, linkers may comprise a number of residues or subunits that mediate interactions between the biomolecule and one or more target ligands. In some embodiments, these two or more residues or subunits may be cleaved from one another with high specificity in order to sever the interaction between the biomolecule and one or more target ligands. Further variations in ligand composition and their uses are known.

Regardless of linker configuration, methods of selecting a biomolecule suitable for modification by the one or more linkers are known and generally comprise using criteria including those identified above or elsewhere herein.

### Step 1c: Selection of Biomolecules Suitable for Modification by Intramolecular Modification

In some embodiments, a biomolecule's structure may be modified by one or more intramolecular modifications that confer altered functionality on the resultant biomolecule variant. Regardless of type of intramolecular modifications introduced, methods of selecting a biomolecule suitable for such modifications are known and generally comprise using criteria including those identified above or elsewhere herein.

### Step 1d: Selection of Biomolecules Suitable for Modification by Addition of Compounds for Medical Treatment

In some embodiments, a biomolecule's structure may be modified by the addition of one or more compounds of medical treatment that affect the function of the resultant biomolecule-compound complex (i.e. the variant). Regardless of species of compound for medical treatment, methods of selecting a biomolecule that is a suitable target for such compounds are known and generally comprise using criteria including those identified above or elsewhere herein.

### Step 2: Obtaining Structure of Biomolecules

According to embodiments, the present processes for designing and producing biomolecules may also comprise a step of obtaining at least one structure of the at least one biomolecule. Biomolecule structures may be obtained, for example, by methods that include direct elucidation of biomolecule structure through crystallography, cryogenic electron microscopy (cryo-EM), nuclear magnetic resonance (NMR) spectroscopy, or electron paramagnetic resonance (EPR) spectroscopy. In some embodiments, methods of obtaining at least one structure of the at least one biomolecule may include prediction modelling, such as homology modeling based on similar biomolecules (e.g. homologs and paralogs) with known structures, computational prediction modelling (e.g. AlphaFold), and computational docking studies (e.g. where molecular dynamics simulations are used to introduce a ligand into an apo structure to predict a ligand-bound structure). In other embodiments, methods of obtaining at least one structure of the at least one biomolecule may include obtaining known structures from structural databases (e.g. the Protein Data Bank archive). Some embodiments will result in the obtainment of at least one three-dimensional structure of the biomolecule. Some embodiments will result in the obtainment of at least one full atomic three-dimensional structure of the biomolecule. Preferred embodiments will result in the obtainment of full atomic three-dimensional structures of the biomolecule in at least two different states (e.g. an apo and at least one ligand-bound state or, alternatively, two different ligand-bound states).

In the first example that follows, structural models of Streptococcus pneumoniae MalX were obtained in its apo and MOS-bound states from the Protein Data Bank archive (2XD2 and 2XD3, respectively).

In the second example that follows, structural models of Yersinia enterocolitica TogB were obtained in its apo, 4,5-unsaturated digalacturonic acid-bound state, digalacturonic acid-bound state, and trigalacturonic acid-bound states were obtained from the Protein Data Bank archive (2UVG, 2UVH, 2UVI, and 2UVJ, respectively).

In the third example that follows, structural models of Escherichia coli EF-Tu were obtained by several methods including prediction modelling. Specifically, a structural model of 25 Escherichia coli EF-Tu was obtained in its GDP-bound state from the Protein Data Bank archive (1EFC) and a structural model of Escherichia coli EF-Tu in its GTP bound state was homology modeled using a structural model of Thermus aquaticus EF-Tu in its 5′-guanylyl imidodiphosphate (GDPNP)-bound state from the Protein Data Bank archive (lEFT), wherein GDPNP was manually converted to GTP.

Once at least one structure of the at least one biomolecule has been obtained, the molecular dynamics of the at least one structure may be simulated.

### Step 3: Performing Molecular Dynamics Simulations

According to embodiments, the present processes for designing and producing biomolecules may also comprise a step of simulating the molecular dynamics of at least one structure of a biomolecule to generate dynamic information about at least one position within the at least one structure. Molecular dynamics simulations may be used to generate dynamic information about how the structure changes over time. Molecular dynamics simulations may comprise various conditions (e.g. temperature, solvation, ionic strength) and may be performed over various timeframes (e.g. 10 ns, 100 ns) and time resolutions (e.g. 2 fs steps). Molecular dynamics simulations may comprise various computational parameters (e.g. CHARMM, AMBER, GROMOS, GLYCAM) and may be run using various software packages (e.g. NAMD, AMBER, GROMACS) as is known. In some embodiments, the molecule dynamic simulations of the at least one structure may generally comprise the steps of (i) solvating at least one structure of a biomolecule by adding a water box; (ii) adding salt to approximate ionic environment of the biomolecule's target environment, cellular environment, or appropriate buffer; (iii) minimizing the energy of the system by applying force fields over a number of simulation steps; (iv) equilibrating the system; (v) raising the temperature of the system to approximate the biomolecule's target environment, cellular environment, or appropriate buffer over a number of simulation steps; and (vi) simulating the system over a number of steps to generate dynamic information about at least one position within the at least one structure.

Methods of performing such molecular dynamics simulations are known for each species of biomolecule, including for polypeptide structures, nucleic acid structures (CASE, D. A., et al., The Amber biomolecular simulation programs. J. Comput. Chem., 26 (2005), pages 1668-88), lipid structures (CALLUM J., et al., Journal of Chemical Theory and Computation. 2014 10 (2), pages 865-79), and carbohydrate structures (CASE, D. A., et al., The Amber biomolecular simulation programs. J. Comput. Chem., 26 (2005), pages 1668-88).

In the first example that follows, each structure of the Streptococcus pneumoniae MalX polypeptide was solvated with a TIP3P water box protruding 20 Å from any point on the protein and brought to a concentration of 100 mM NaCl. The potential energy of each model was minimized using AMBERFF99S force fields for 10 000 steps. The system was equilibrated and subsequently heated to 300 K for 10 000 steps each. Finally, MD simulations were performed for 100 ns at a step size of 2 fs using Langevin dynamics to maintain temperature.

In the second example that follows, each structure of the Yersinia enterocolitica TogB was solvated with a TIP3P water box protruding 10 Å from any point on the protein and neutralized with a suitable concentration of Na⁺ ions. The potential energy of each model was minimized using AMBERFF99S and GLYCAM force fields for 10 000 steps. The system was equilibrated and subsequently heated to 300 K for 10 000 steps each. Finally, MD simulations were performed for 100 ns at a step size of 2 fs using Langevin dynamics to maintain temperature.

In the third example that follows, each structure of the Escherichia coli EF-Tu was hydrogenated and solvated with a TIP3P water box protruding 10 Å from any point on the protein. The potential energy of the systems was minimized using a two-fold iterative process minimizing the potential energy of water molecules followed by a minimization of the protein using CHARMM 27 parameters for 10 000 steps. Each EF-Tu conformation was subsequently neutralized with NaCl ions followed by a final minimization of the entire system for 100 000 steps. Each system was then equilibrated to 300 K and 350 K for 300 000 steps. Finally, MD simulations were performed at 300 K using velocities for the 350 K equilibration and atomic positions from the 300 K equilibration for 100 ns at a step size of 2 fs using Langevin dynamics to maintain temperature.

Once molecular dynamics simulations have been performed on the at least one structure of the biomolecule, at least one score can be calculated for at least one position within the at least one structure of the biomolecule.

### Step 4: Calculating F_{score}

According to embodiments, the present processes for designing and producing biomolecules may also comprise a step of using dynamic information about at least one position within at least one structure of a biomolecule to calculate a score for the at least one position. The term “F_{score}” is used herein to refer to a numerical value that represents the suitability of a target position for modification. In some embodiments, F_{score} may be calculated from one or more sets of dynamic information. Where more than two sets of dynamic information are considered, it may be desirable to weigh each set compared to the other sets. In some embodiments, the F_{score} may be calculated from sets of dynamic information that indicate conformational and/or environmental changes within the structure. Such changes may occur from one distinct state to the next or occur temporarily between two distinct states (e.g. between an apo and ligand-bound state or, alternatively, between two or more ligand-bound states). In such embodiments, it may be desirable to quantify such changes by calculating the difference in dynamic information between one state and another. The one or more sets of dynamic information that should be factored into a useful F_{score}, as well as how those sets should be weighed relative to the others, is readily appreciated.

For example, in some embodiments, the F_{score} may be calculated from dynamic information comprising relative distances between atoms within the at least one position and atoms within at least one other position. In the case of polypeptides, such atoms may include, for example, C_{α} atoms within different amino acid residues. In the case of nucleic acids, such atoms may include, for example, C1′ atoms within different nucleotide residues. In the case of lipids, such atoms may include, for example, the phosphorus atom within the phosphorous head of a phospholipid. In the case of carbohydrates, such atoms may include, for example, the first carbon atom according to suitable nomenclature standards for carbohydrates.

In other embodiments, the F_{score} may be calculated from dynamic information comprising changes in dihedral angle across atomic bonds within the at least one position (e.g. backbone dihedral angles within the residues or subunits of a polymer). In the case of polypeptides, such dihedral angles may comprise, for example, the <(across atoms C, N, C_{α}, and C) or W (across atoms N, C_{α}, C, and N) angles within different amino acid residues. In the case of nucleic acids, such dihedral angles may comprise, for example, the ϕ (across atoms O3′, P, O5′, and C5′), ψ (across atoms P, O5′, C5′, and C4′), y (across atoms O5′, C5′, C4′, and C3′), δ (across atoms C5′, C4′, C3′, and O3′), ε (across atoms C4′, C3′, O3′, and P), ζ (across atoms C3′, O3′, P, and O5′), or χ (across atoms C2′, C1′, N1 or N9 (pyrimidine or purine), and C2 or C4 (pyrimidine or purine)) angles within different nucleotide residues. In the case of carbohydrates, such dihedral angles may comprise, for example, the φ (C2A, C1A, O, and C2B) and ψ (C1A, O, C2B, and C3B) angles between two sugar subunits. Similar dihedral angles are known for lipids (see e.g. PEZESHKIAN, W., et al. Lipid Configurations from Molecular Dynamics Simulations. Biophysical Journal, 2018 Apr. 24, 114(8), page 1895-907). In some embodiments, dihedral angles may be used to construct a Ramachandran plot. In such embodiments, the Ramachandran plot may be transformed into a matrix and the sum of the difference between the matrices for the at least one structure may be used to quantify the change in dihedral angles within that at least one position. In some embodiments, a 180×180 matrix may be used. Other methods of calculating dihedral angles for at least one position within a biomolecule are known.

In other embodiments, the F_{score} may be calculated from dynamic information comprising surface accessible solvent areas of atoms within the at least one position.

In other embodiments, the F_{score} may be calculated from dynamic information comprising relative distances between atoms within the at least one position and atoms within an at least one ligand bound to the at least one structure. In some embodiments, distances between atoms and the at least one ligand may be measured for the ligand bound simulation directly. In other embodiments, the simulated structure of the ligand bound conformation may be aligned to the simulated apo structure to measure the distance from each amino acid to the ligand. In yet other embodiments, a cut-off value may be used to minimise impact of modifications at the target position on ligand binding properties. Such a cut-off may be used to eliminate all positions that are a distance of 5 Å or less from the ligand.

In other embodiments, the F_{score} may be calculated from dynamic information comprising relative distances between atoms within the at least one position and atoms within at least one functional group or residue located within the at least one structure that demonstrates intrinsic spectroscopic properties (e.g. tryptophan residues in the case of polypeptides). In some embodiments there may be two or more functional groups or residues located within the at least one structure that demonstrate intrinsic spectroscopic properties. In such embodiments, the distance between atoms within the at least one structure and atoms within each of the two or more functional groups or residues may be averaged.

In other embodiments, the F_{score} may be calculated from dynamic information comprising root mean square fluctuations (RMSF) of atomic positions within the at least one position.

In other embodiments, the F_{score} may be calculated from dynamic information comprising alterations in intramolecular arrangements within the at least one position. In the case of amino acids, such altered arrangements may include non-Watson Crick face interactions like Hoogsteen and sugar edge interactions. In the case of carbohydrates, such altered arrangements may include degrees of polymerization, ring configurations, and steric configurations.

In other embodiments, the F_{score} may be calculated from static information comprising conservation score of the atom, functional group, or residue within the at least one position. In some embodiments, a cut-off value may be used to minimise impact of modifications at the target position on ligand binding properties. Such a cut-off may be used to eliminate all positions that have a high conservation score. In some embodiments a cut-off value may be a ConSurf score of 1 for polypeptides (GLASER, F., et al., ConSurf: Identification of Functional Regions in Proteins by Surface-Mapping of Phylogenetic Information. Bioinformatics, 19 (2003), pages 163-64; LANDAU, M., et al., ConSurf 2005: the Projection of Evolutionary Conservation Scores of Residues on Protein Structures. Nucleic Acids Research, 33 (2005), pages W299-W302).

According to embodiments, the F_{score} may be calculated by averaging [symbol] [symbol] all dynamic information for each structure, taking the absolute value | of the difference between dynamic information for each structure, and normalizing the resultant F_{score} compared to the maximum (v) F_{score} obtained or a pre-determined reference score. In some embodiments, a multiplication factor may be used to adjust the relative weight of each set of dynamic information used to calculate the F_{score}. In some embodiments, each residue or subunit within the biomolecule may be assigned an F_{score} between 0 and n, where n is the number of sets of dynamic information used to calculated the F_{score}. For example, each residue or subunit within the biomolecule may be assigned an F_{score} between 0 and 5. By way of example, an F_{score} for each position within a biomolecule may be calculated using Equation 1, wherein the five stets of dynamic information generated from an apo and ligand-bound structure of the biomolecule considered therein is identified according to the variables defined in Table 2.

$${F}_{s c o r e} = \frac{\vert 〈 {S}_{L r} 〉 - 〈 {S}_{A r} 〉 \vert}{∨ \vert 〈 {S}_{L r} 〉 - 〈 {S}_{A r} 〉 \vert} + \frac{\vert 〈 {T}_{L r} 〉 - 〈 {T}_{A r} 〉 \vert}{∨ \vert 〈 {T}_{L r} 〉 - 〈 {T}_{A r} 〉 \vert} + \frac{\vert 〈 {L}_{L r} 〉 - 〈 {L}_{A r} 〉 \vert}{∨ \vert 〈 {L}_{L r} 〉 - 〈 {L}_{A r} 〉 \vert} + \frac{\vert 〈 {F}_{L r} 〉 - 〈 {F}_{A r} 〉 \vert}{∨ \vert 〈 {F}_{L r} 〉 - 〈 {F}_{A r} 〉 \vert} + \frac{\sum \vert [ {B}_{L r} ] - [ {B}_{A r} ] \vert}{∨ \sum \vert [ {B}_{L r} ] - [ {B}_{A r} ] \vert} \qquad ( Equation 1 )$$

TABLE 2
Dynamic Information Variables in Equation 1

| Variable | Dynamic Information of Each Position |
|---|---|
| S_{Lr} | Solvent accessibility of position in ligand-bound |
| | structure |
| S_{Ar} | Solvent accessibility of position in apo structure |
| T_{Lr} | Distance between Tryptophan residue and position in |
| | ligand-bound structure |
| T_{Ar} | Distance between Tryptophan residue and position in |
| | apo structure |
| L_{Lr} | Distance between Ligand and position in ligand-bound |
| | structure |
| L_{Ar} | Distance between Ligand and position in apo structure |
| F_{Lr} | RMSF of position in ligand-bound structure |
| F_{Ar} | RMSF of position in apo structure |
| B_{Lr} | Backbone dihedral angle of position in ligand-bound |
| | structure |
| B_{Ar} | Backbone dihedral angle of position in apo structure |

Other methods of calculating a score for at least one position within a biomolecule based on dynamic information will be appreciated from the present disclosure.

In the examples that follow, F_{score} were calculated from sets of dynamic information that are known in the art to influence the spectral properties of a conjugated fluorophore either by directly describing changes in potential conjugated fluorophore interactions or describing spatial changes in the conjugated fluorophore relative to the rest of the polypeptide. These sets of dynamic information were given equal weight for illustration purposes only. It is understood that the examples that follow, as well as the embodiments disclosed herein, are not exhaustive and that further embodiments are appreciated.

Step 5: Comparing F_{score} with Reference to Select Site

According to embodiments, the present processes for designing and producing biomolecules may also comprise a step of comparing a score for at least one position within the at least one structure of a biomolecule with at least one reference score to identify at least one target position within the biomolecule suitable for modification.

Whether any particular F_{score} indicates a suitable target position for modification depends on the intended functionality of the resultant biomolecule variant. For example, in some embodiments, in the case of an F_{score} calculated from dynamic information that tends to indicate a position within the structure that is subject to local conformational changes, a high F_{score} will represent a more mobile position while a low F_{score} will represent a more stable position. If the indented functionality of the resultant variant benefits from modification of a more mobile position within the biomolecule, a high F_{score} will indicate a target position that is more likely to be suitable. In other embodiments where an allosteric target position is desired, a higher F_{score} may indicate a greater distance between the target position and the ligand binding pocket. In other embodiments where a peristeric or endosteric target position is desired, a higher F_{score} may indicate a shorter distance between the target position and the ligand binding pocket. In some embodiments where a solvent-accessible target position is desired, a higher F_{score} may indicate a more solvated target position. As these examples demonstrate, the relationship between F_{score}, target positions, and the intended functionality of the resultant biomolecule variant is readily apparent.

According to embodiments, the quality of any particular F_{score} can be obtained by comparing the F_{score} to a reference score. The reference score may either be an F_{score} for another position or a pre-determined value. In some embodiments, a more suitable target position can be identified by selecting an F_{score} that is closest to the pre-determined value or by selecting an F_{score} that is greater than all of the other F_{score}s. The higher the relative or absolute value of the F_{score}, the more likely the target position is to be suitable for modification. In the examples that follow, higher F_{score}s represent positions that are likely to influence the spectral properties of a conjugated fluorophore either by directly describing changes in potential conjugated fluorophore interactions or describing spatial changes in the conjugated fluorophore relative to the rest of the polypeptide.

Once a score for at least one position within the at least one structure of a biomolecule has been compared with at least one reference score and at least one target position within the biomolecule suitable for modification has been identified, that at least one target position may be modified to produce a rationally-designed biomolecule.

### Step 6: Producing Rationally-Designed Biomolecules

According to embodiments, the present processes for designing and producing a biomolecule may also comprise a step of modifying at least one target position within the biomolecule. Methods of biomolecule modification are known in the art. For example, modified biomolecules may be produced by site-specifically introducing a modification by total synthesis, semi-synthesis, or gene fusions (see, for example, ADAMS et al., Nature 39:694-697, 1991; BRUNE et al., Biochemistry 33:8262-8271, 1994; GILARDI et al., Anal. Chem. 66:3840-3847, 1994; GODWIN et al., J. Am. Chem. Soc. 40 118:6514-6515, 1996; MARVIN et al., Proc. Natl. Acad. Sci. U.S.A. 94:4366-4371, 1997; POST et al., J. Biol. Chem. 269:12880-12887, 1994; ROMOSER, J. Biol. Chem. 272: 13270-13274, 1997; THOMPSON et al., J. Biomed. Op. 1:131-45 137, 1996; WALKUP et al., J. Am. Chem. Soc. 119:5445-5450, 1997). In other embodiments directed to polypeptides, modifications are made by site-specific mutagenesis of nucleotides in the DNA encoding the polypeptide, thereby producing DNA encoding the modification, and thereafter expressing the DNA in recombinant cell culture. Techniques for making substitution mutations at predetermined sites in DNA having a known sequence are well known, for example M13 primer mutagenesis and PCR mutagenesis. Other production methods are known in the art.

### Example 1

Molecular Dynamics Simulations of Streptococcus Pneumoniae MalX. Structural models of MalX in its apo and MOS-bound state were obtained from protein data bank (2XD2 and 2XD3, respectively (ABBOTT, D. W. et al. The Molecular Basis of Glycogen Breakdown and Transport in Streptococcus Pneumoniae. Molecular Microbiology, 77 (2010), pages 183-99)) and used for molecular dynamics simulations. Each model of the protein was solvated with a TIP3P water box protruding 20 Å from any point on the protein and brought to a concentration of 100 mM NaCl. The potential energy of each model was minimized using AMBERFF99S force fields for 10 000 steps. The system was equilibrated and subsequently heated to 300 K for 10 000 steps each. Finally, molecular dynamics simulations were performed for 100 ns at a step size of 2 fs using Langevin dynamics to maintain temperature.

Calculating F_{score}. Dynamic information comprising the backbone flexibility, backbone dihedral angles, distance to ligand, distance to tryptophan residues, and solvent accessibility for each amino acid position were measured to develop a ranking score (FIG. 1). These sets of dynamic information were chosen as small dynamic features of the amino acid position that would have the largest impact on the environment of the conjugated fluorophore. Only amino acids with a conservation score of 1 from the ConSurf server (GLASER, F., et al., ConSurf: Identification of Functional Regions in Proteins by Surface-Mapping of Phylogenetic Information. Bioinformatics, 19 (2003) 163-164; LANDAU, M., et al., ConSurf 2005: the Projection of Evolutionary Conservation Scores of Residues on Protein Structures. Nucleic Acids Research, 33 (2005) W299-W302) and further than 5 Å from the ligand binding site were assigned an F_{score} to ensure there would be no impact on ligand binding properties. Flexibility was determined by measuring the Root Mean Square Fluctuation (RMSF) of the Cα atoms of each amino acid over the course of both the ligand bound (FLr) and apo (FAr¬) simulations (Equation 1). Backbone dihedral angles were measured by determining the ϕ (between atoms C, N, Cα, and C) or W (between atoms N, Cα, C, and N) angles and then using these angles to construct a Ramachandran plot. The Ramachandran plot was then transformed into a 180×180 matrix (FIG. 3) and the sum of the difference between the matrices for the ligand bound (BLr) and apo (BAr) conformation was used to determine the conformational change in the backbone dihedrals at that residue (Equation 1). Distances from each amino acid to the ligand were measured for the ligand bound simulation (LLr) directly, whereas the structure of the ligand bound conformation was aligned to the apo simulation to measure the distance from each amino acid to the ligand (LAr) (Equation 1). Tryptophan distances were measured for both ligand-bound (TLr) and apo (TAr) (Equation 1). In circumstances where there is more than one tryptophan the distances between an amino acid and each tryptophan were averaged to obtain an average amino acid to tryptophan distance. Last, the solvent accessibility of each amino acid was determined and compared between the ligand bound (SLr) and apo (SAr) conformations (Equation 1). The values measured were averaged [symbol] [symbol] and the absolute value ∥ of the difference between ligand bound and apo conformations was normalized compared to the maximum (∨) value obtained (Equation 1). Using this approach each amino acid that was under consideration could be assigned an F_{score} from 0 to 5, with a higher score indicating a better candidate for fluorophore conjugation.

Biosensor construct design. All MalX variants were engineered to lack the signal secretion peptide and lipoprotein attachment motif found in wild-type malX as previously described (Abbott et al. 2010). malX variants were synthesized with flanking 5′ NheI and 3′ XhoI restriction sites and subcloned into pET28a yielding pET28a::malX A128C, pET28a::malX A174C, pET28a::malX T243C, and pET28a::malX E312C (BioBasic Canada Inc.). Criteria for selection of these mutants was guided by the present processes. All constructs encoded an N-terminal poly-Histidine tag fusion.

Expression of MalX variants. Escherichia coli BL21(DE3) gold cells (Agilent) transformed with either pET28a::malX A128C, pET28a::malX A174C, pET28a::malX T243C, or pET28a::malX E312C were used to inoculate LB media (10 g/L typtone, 5 g/L yeast extract, 10 g/L NaCl) supplemented with 50 mg/L kanamycin to an optical density at 600 nm (OD₆₀₀)≈0.1. After growth at 37° C. with 200 RPM shaking, isopropyl β-D-1-thiogalactopyranoside (IPTG) was added to a final concentration of 1 mM when OD₆₀₀ reached approximately 0.6. Cultures were grown for an additional 3 hours, cells harvested by centrifugation (5000×g, 10 minutes, 4° C.), flash frozen, and stored at −80° C. for further use.

Purification of MalX variants. Cell pellets were resuspended in 7 mL/g of Buffer A (20 mM Tris pH 8.0 at 4° C., 0.5M NaCl, 10 mM imidazole, 7 mM β-mercaptoethanol (BME), 1 mM phenylmethylsulfonylfluoride (PMSF)), and lysozyme added to a final concentration of 1 mg/mL prior to a 30-minute incubation at 4° C. Sodium deoxycholate was added to a final concentration of 12.5 mg per gram of cells, and the suspension was sonicated (Branson Sonifier 450) on ice for 1 min at 50% output and 60% duty cycle (repeated twice, with a 5 min break between cycles). The mixture was centrifuged (3000 xg, 30 minutes, 4° C.) to pellet insoluble debris, followed by additional centrifugation to produce S30 supernatant (30 000 xg, 45 minutes, 4° C.). S30 supernatant was applied to Ni²⁺-Sepharose resin in a batch-chromatography setup (3 mL resin per 1 g of cells opened) and incubated 30 minutes at 4° C. Ni²⁺-Sepharose resin was collected by centrifugation (500×g, 2 minutes, 4° C.), and supernatant decanted. The collected Ni²⁺-Sepharose resin was washed 3 times with 10 resin-volumes of Buffer A, followed by 4 washes with 10 resin-volumes Buffer B (Buffer A with 20 mM imidazole). Bound protein was eluted six times with 1 resin-volume Buffer C (Buffer A with 250 mM imidazole). Samples were analyzed via sodium dodecyl sulfate polyacrylamide gel electrophoresis (SDS-PAGE) stained with Coomassie Brilliant Blue, and elutions containing each MalX variant were pooled and dialyzed at 4° C. into Buffer D for labeling (20 mM Tris pH 7.5 @ 20° C., 0.5M NaCl, 30 mM imidazole, 20 mL sample into 1 L Buffer D, dialysis tubing molecular weight cut-off 12.4 kDa, 3 changes). Purified protein was flash-frozen with liquid nitrogen and stored at −80° C. for further use, and purification yields were typically 100 mg of protein per liter of culture.

Fluorescent labeling of MalX variants. Labeling reactions were performed essentially as described previously (SMITH et al. 2017). MalX variants (100 μM concentration in labeling reaction, 15 mL reaction volume) was incubated with 3.5 mL Ni²⁺-Sepharose resin in Buffer D. Five-fold molar excess of either 7-diethylamino-3-(4′-maleimidylphenyl)-4-methylcoumarin (CPM, from 20 mM stock in dimethyl sulfoxide (DMSO), Biotium), N-(7-dimethylamino-4-methylcoumarin-3-yl)maleimide (DACM, from 20 mM stock in dimethylformamide (DMF), Invitrogen), N-[2-(dansylamino)ethyl]maleimide (Dansyl, from 25 mM stock in DMSO, Sigma Aldrich), fluorescein-5-maleimide (Fluorescein, from 50 mM stock in DMSO, Biotium), 7-diethylamino-3-[N-(2-maleimidoethyl)carbamoyl]coumarin (MDCC, from 20 mM stock in DMF, Sigma Aldrich), N-(1-pyrene)maleimide (Pyrene, from 20 mM stock in DMF, Sigma Aldrich), or Rhodamine Red C2 maleimide (Rhodamine Red, from 20 mM stock in DMF, Invitrogen) was added dropwise to the labeling mixture corresponding to 500 μM in the labeling reaction. The labeling reaction was subsequently incubated on an end-over-end mixer at room temperature for 2 hours at room temperature, followed by 12 hours at 4° C. Ni²⁺-Sepharose resin was collected by centrifugation (500×g, 2 minutes, 4° C.) and supernatant decanted. Resin was washed six times with three resin-volumes of Buffer D, followed by six elutions with Buffer E (Buffer D with 250 mM imidazole). Labeling procedure samples were analyzed via SDS PAGE, and the respective gels visualized under UV light (312 nm) or stained using Coomassie Brilliant Blue. Elutions containing each labeled MalX variant were pooled and dialyzed into 50 mM Tris pH 7.5 @ 20° C. (20 mL sample in 1 L Buffer, 4 changes, molecular weight cut-off 12.4 kDa). The protein recovery from the labeling procedure was ˜50% and labeling efficiencies were typically 70% to >90%. Concentration of MalX A128C-MDCC, and labeling efficiency of MalX A128C-MDCC was calculated as described below (Equations 2, 3, and 10). The following parameters were used: 0.164 was a correction factor accounting for MDCC absorption at 280 nm (BRUNE et al. 1994; SMITH et al. 2017), ε280, MalX=61 310 M⁻¹ cm⁻¹ was the calculated extinction coefficient based on protein primary sequence (GASTEIGER et al. 2005), L is instrument path length in cm, and ε430, MDCC=46 800 M⁻¹cm⁻¹ (BRUNE et al. 1994; SMITH et al. 2017).

$$[ MalXA 128 C ] = \frac{{A}_{280} - ( {A}_{430} \times 0.164 )}{{ε}_{280 , MalX} \times L} \qquad ( Equation 2 )$$

$$[ MDCC ] = \frac{{A}_{430}}{{ε}_{430 M D C C} \times L} \qquad ( Equation 3 )$$

Protein concentration for MalX A128C-CPM, MalX A128C-DACM, MalX A128C-Dansyl, MalX A128C-Fluorescein, MalX A128C-Pyrene, and MalX A128C-Rhodamine Red were determined via densitometry analysis (ImageJ (SCHNEIDER, C. A., et al., NIH Image to ImageJ: 25 years of image analysis, Nature Methods, 9 (2012) 671)) of SDS-PAGE gels using MalX A128C-MDCC samples to form a standard curve. Concentrations of the respective conjugated fluorescent labels were calculated from spectroscopic data according to manufacturer provided extinction coefficients (Equations 4-10): 384, CPM=33 000 M⁻¹cm⁻¹, 384, DACM=27 000 M⁻¹ cm⁻¹, 340, Dansyl=4 300 M⁻¹ cm⁻¹, ε494, Fluorescein=68 000 M⁻¹ cm⁻¹, ε339, Pyrene=38 000 M⁻¹cm⁻¹, ε560, Rhodamine Red=119 000 M⁻¹cm¹.

$$[ C P M ] = \frac{{A}_{384}}{{ε}_{384 , C P M} \times L} \qquad ( Equation 4 )$$

$$[ DACM ] = \frac{{A}_{384}}{{ε}_{384 , DACM} \times L} \qquad ( Equation 5 )$$

$$[ Dansyl ] = \frac{{A}_{340}}{{ε}_{340 , Dansyl} \times L} \qquad ( Equation 6 )$$

$$[ Fluorescein ] = \frac{{A}_{494}}{{ε}_{494 , Fluorescein} \times L} \qquad ( Equation 7 )$$

$$[ Pyrene ] = \frac{{A}_{339}}{{ε}_{339 , Pyrene} \times L} \qquad ( Equation 8 )$$

$$[ Rhodamine Red ] = \frac{{A}_{560}}{{ε}_{560 , Rhodamine Red} \times L} \qquad ( Equation 9 )$$

$$Labeling efficiency = \frac{[ Fluorophore ]}{[ M a l X A 128 C ]} \times 100 \% \qquad ( Equation 10 )$$

Carbohydrates and α-amylase. Maltotriose was purchased from Megazyme (O-MAL3); Maltose (M5885), water soluble starch (S9765), and Bacillus licheniformis α-amylase (A3403) was purchased from Sigma Aldrich.

Equilibrium fluorescence experiments. Fluorescence spectrophotometry was performed using a Quanta Master 60 Fluorescence Spectrometer (Photon Technology International, all experiments utilized 1 nm step size, 1 s integration). For experiments in FIG. 4, excitation slit widths were 1 nm, emission slit widths were 2 nm, excitation wavelength was 420 nm, and emission wavelength was 425-575 nm. For data presented in FIG. 5, excitation and emission wavelengths were the same as in FIG. 4, except excitation slit widths were 4 nm, emission slit widths were 8 nm. To obtain K_{D}, a hyperbolic function (Equation 11) was fit to the data in GraphPad Prism v. 5.0, where Y is the absolute value of the fluorescence change at each [M3], Bₘₐₓ is the maximum specific binding in the same units as Y extrapolated to very high ligand concentrations, and K_{D} is the equilibrium binding constant.

$$Y = \frac{{B}_{max} \times [ M 3 ]}{{K}_{D} + [ M 3 ]} \qquad ( Equation 11 )$$

For data presented in Table 3, protein concentration was 20 nM (except the MalX A128C-Dansyl conjugate, which was assayed at 1 μM due to lower extinction coefficient), and the excitation wavelength listed in Table 3 was utilized. Emission scans were generally performed in windows of 150 nm and included the designated Table 3 emission maxima wavelengths. ΔI was determined as follows:

$$Δ I = \frac{maximum emission ( MOS bound ) - maximum emission ( apo )}{maximum emission ( apo )} \times 100 \% \qquad ( Equation 12 )$$

TABLE 3
Response to M3 by various MalX A128C-fluorophore conjugates.

| | Response | | |
|---|---|---|---|
| Fluorophore | ΔI | Ex. max (nm) | Em. max (nm) |
| Pyrene | −10% | 340 | 380 |
| MDCC | −20% | 420 | 465 |
| CPM | −20% | 385 | 465 |
| DACM | −10% | 380 | 465 |
| Fluorescein | +6% | 495 | 515 |
| Dansy1 | +8% | 340 | 520 |
| Rhodamine Red | +20% | 560 | 580 |

ΔI indicates change in peak fluorescence intensity upon addition of 50-fold molar excess M3.

Rapid-kinetics measurements. Rapid kinetics experiments were performed in a KinTek SF-2004 stopped-flow apparatus (KinTek Corp.) at 20° C. An excitation wavelength of 420 nm was used for all rapid kinetics experiments, and fluorescence emission was measured through a 450 nm long-pass filter. Individual fluorescence time-courses were fit with a one exponential function (Equation 13), where F is fluorescence observed at time t, F_{∞} is final fluorescence, A is signal amplitude, and kₐₚₚ is apparent rate (TableCurve, Systat Software).

F=F_{∞}+A₁×exp(−kₐₚₚt) (Equation 13)

Microplate reader experiments. Microplate reader experiments were performed using a SpectraMax i3x plate reader (Molecular Devices, Fluorescence: Kinetic mode, 420 nm excitation (9 nm range), 465 nm emission (15 nm range), PMT gain: high, 6 flashes per read, sampling interval: 1 minute) in a 96 well plate. Individual fluorescence time-courses were fit using a one-exponential function as described vide supra (Equation 13), and data was plotted using GraphPad Prism v. 5.0.

Results of fluorophore conjugation sites in MalX. As proof-of-concept the present processes were used to rank select an optimal fluorophore conjugation site in MalX for development of a novel MOS-detecting biosensor. The present processes were developed to take into consideration structural dynamic features of biomolecules that, for example, may differ between two distinct states. The properties selected were RMSF, surface accessible solvent area, backbone flexibility, and distance to ligand or tryptophan. All five properties were selected for their potential influence on the spectral properties of a conjugated fluorophore, either by directly describing changes in potential conjugated fluorophore interactions or describing spatial changes in the conjugated fluorophore relative to the rest of the protein.

Both the apo and MOS-bound MalX states were each subjected to 100 ns molecular dynamics simulations and candidate amino acid positions were subjected to the present processes to determine amino acid F_{score} (FIG. 2). Amino acids were considered candidates if they were given a Consurf score of 1 indicating lack of sequence conservation (GLASER, F., et al., ConSurf. Identification of Functional Regions in Proteins by Surface-Mapping of Phylogenetic Information. Bioinformatics, 19 (2003) 163-164; LANDAU, M., et al., ConSurf 2005: the Projection of Evolutionary Conservation Scores of Residues on Protein Structures. Nucleic Acids Research, 33 (2005) W299-W302). From the F_{score} data four positions were chosen to mutate to cysteine and subsequently conjugate with a thiol-reactive fluorophore. Positions A128 and A174 were chosen as two of the highest overall scorers (F_{score} 2.7 and 2.1 respectively), and positions T243 and E312 (F_{score} 1.7 and 1.9 respectively) were chosen as mid-level scorers to test the robustness of F_{score} (FIG. 2). Of the labeling positions chosen, the strongest contributor to F_{score} for each mutant was changes in backbone dihedral angles (A128), changes in RMSF (A174), a combination of factors (T243), and change in proximity to ligand (E312)—these mutants can therefore be used to examine the effectiveness of these parameters in the F_{score} algorithm.

Candidate biosensor response to M3, fluorescence spectrophotometry. Initially each of the four MalX variants were conjugated with MDCC, a diethylaminocoumarin previously used in the construction of other solute-binding based biosensors (BRUNE, M., et al., Direct, Real-Time Measurement of Rapid Inorganic Phosphate Release Using a Novel Fluorescent Probe and its Application to Actomyosin Subfragment 1 ATPase. Biochemistry, 33 (1994) 8262-8271; HANES, J. W., et al., Construction of a Thiamin Sensor from the Periplasmic Thiamin Binding Protein. Chemical Communications, 47 (2011) 2273-2275; HIRSHBERG, M., et al., Crystal Structure of Phosphate Binding Protein Labeled with a Coumarin Fluorophore, a Probe for Inorganic Phosphate. Biochemistry, 37 (1998) 10381-10385; KUNZELMANN, S., WEBB, M. R., A Biosensor for Fluorescent Determination of ADP with High Time Resolution. Journal of Biological Chemistry, 284 (2009) ISSN 33130-33138; SALINS, L. L., et al., A Fluorescence-Based Sensing System for the Environmental Monitoring of Nickel Using the Nickel Binding Protein from Escherichia Coli. Analytical and Bioanalytical Chemistry, 372 (2002) 174-180; SMITH, D. D., et al., Streamlined Purification of Fluorescently Labeled Escherichia Coli Phosphate-Binding Protein (PhoS) Suitable for Rapid-Kinetics Applications. Analytical Biochemistry: Methods in the Biological Sciences, 537 (2017) 106-113). Each MalX variant labeled with MDCC was examined using fluorescence spectrophotometry, directly measuring the change in fluorescence response to M3. The highest CINC scorer, A128, and a mid-CINC-scorer T243 displayed a 20% and 30% fluorescence decrease upon the addition of M3, respectively (FIG. 4 A, E). The MalX variants with MDCC conjugated at A174C and E312C did not show a fluorescence change upon binding M3 (FIG. 4 C, G). In the presence of non-specific ligand (maltose) little to no fluorescence change was observed for all the variants (FIG. 4 B, D, F, H). Therefore, MalX A128C-MDCC and MalX T243C-MDCC are able to selectively detect M3 via a fluorescence change specific to MOS binding. Results for the four tested MalX variants will enable improved selection criteria for the development of future biosensors utilizing the present processes, whereby parameters used to calculate F_{score} can be weighed differently guided by, for example, the success/failure of candidate biosensors response to ligand. For example, the largest contributor to F_{score} for position 128 in MalX was changes in dihedral angles—suggesting that it may be desirable to prioritize changes in dihedral in the F_{score} criteria. Our labelling procedure did not influence the ligand binding affinity of MalX, as a titration of each labeled protein with M3 resulted in K_{D} values of 190±50 nM and 600±200 nM for MalX A128C-MDCC and MalX T243C-MDCC respectively, which are similar to the measured affinity of 800±125 nM for the unlabeled protein (FIG. 5) (ABBOTT et al. 2010). Altogether this displays how robust, sensitive, and specific the present biosensors are for detecting and monitoring sub-μM concentrations of M3 without disruption of the ligand binding properties of wild-type MalX.

Rapid-kinetics of M3 detection by MalX A128C-MDCC. To determine kinetic parameters of M3 binding to MalX A128C-MDCC, the stopped-flow method was used. The stopped-flow is a fluorescence spectrophotometer coupled with a rapid mixing devise, enabling detailed kinetic analysis of rapid biomolecular events. MalX A128C-MDCC was rapidly mixed with increasing concentrations of M3, and the resulting fluorescence time-courses were best fit with a one-exponential function to determine A1 and kₐₚₚ (Equation 13). Consistent with equilibrium-state fluorescence spectrophotometry data (vide supra), addition of M3 to MalX A128C-MDCC resulted in a fluorescence decrease (FIG. 6A). The rate constant (kₒₙ) for M3 binding to MalX A128C-MDCC was determined to be 20±2 M⁻¹s⁻¹ (FIG. 6B), demonstrating rapid M3 detection via our engineered biosensor. Rapid M3 detection by MalX A128C-MDCC makes the biosensor amenable to applications where M3 release is slower than detection by the biosensor (i.e. real-time detection). Rapid kinetics of M3 detection by MalX T243C-MDCC was also examined, however, the resulting fluorescence time-courses did not follow pseudo first-order binding kinetics and this biosensor was not utilized in rapid kinetics assays (data not shown). Although the complex kinetics observed with MalX T243C-MDCC make it less useful for real-time applications, this biosensor has utility in endpoint or equilibrium applications where sensitive MOS detection is required.

MalX A128C can be conjugated to a variety of fluorophores to modulate biosensor spectroscopic properties. To examine the influence of fluorophore species to the present biosensors, MalX A128C was conjugated to a variety of fluorophores with various structural and spectroscopic properties. Ultimately, the impact of different fluorophore families (four) and linker compositions (three) were examined. To address linker composition, the coumarin fluorophores (CPM and DACM) were conjugated to MalX A128C and compared to the previously characterized MDCC conjugate (Table 3). MDCC contains an N-ethylcarbamoyl linker between the coumarin and maleimide group, CPM contains a phenyl linker between the coumarin and maleimide group, and DACM contains no linker (FIG. 7). To examine the impact of fluorophore family on the MalX A128C-based biosensor Dansyl (naphthalene family), Fluorescein (xanthene family), Pyrene (pyrene family), and Rhodamine Red (xanthene family) were conjugated to MalX A128C and their response to a MOS examined (FIG. 8, FIG. 7, Table 3).

All tested fluorophores gave a detectable response to M3, expanding the spectral range of our MOS-biosensor set to report emission maxima of 380 nm to 580 nm. Together, these results demonstrate that a wide variety of fluorophores can probe the altered environment detected via the present processes, enabling rapid development of biosensor libraries with variable spectroscopic properties.

Portable detection of MOS-release from α-amylase using MalX A128C-MDCC. To examine the utility of MalX A128C-MDCC in characterizing enzyme activity, generation of MOS was examined in real-time via Bacillus licheniformis α-amylase-catalyzed degradation of starch. Using the stopped-flow method, all experimental conditions with α-amylase produced a fluorescence signal decrease slower than the rate of M3 binding to MalX A128C-MDCC, and exhibited a dose-dependent relationship with α-amylase concentration (FIG. 9A). The apparent rate of MOS generation was dependent on α-amylase concentration, and fitting with a linear function yielded a rate constant of 0.7±0.1 μM⁻¹min⁻¹ (FIG. 9C), while the amplitude of the signal change was independent of α-amylase concentration (FIG. 9E). To demonstrate portability of our MalX A128C-MDCC biosensor, the α-amylase concentration-dependent rate was confirmed with a commonly-used microplate reader assay (FIG. 9B). In agreement with our stopped-flow assay, the apparent rate of MOS generation was dependent on α-amylase concentration and fitting with a linear function yielded a rate constant of 1.4±0.5 μM⁻¹min⁻¹ (FIG. 9D), while the amplitude of the signal change was independent of α-amylase concentration (FIG. 9F). Together, these findings demonstrate the utility of the MalX A128C-MDCC biosensor in detecting MOS released as a component of the functional cycle of α-amylase.

The present processes were used in the rational design of novel biosensors capable of detecting M3. MalX is capable of binding MOS with DP 3-9 with similar affinity (ABBOTT, D. W., et al., The Molecular Basis of Glycogen Breakdown and Transport in Streptococcus Pneumoniae. Molecular Microbiology, 77 (2010) 183-199), so it is contemplated that MalX A128C-MDCC detects MOS with DP 4-9 as well. Of the four initial positions selected in MalX for substitution and subsequent fluorescent labeling, two resulted in biosensors that gave a response to M3. Of these two, the largest contributor to F_{score} for position A128 was backbone dihedral angles, whereas position T243 was a combination of factors. Of the two positions whose corresponding biosensors did not give rise to M3 detection, the largest contributor to F_{score} for position A174 was RMSF, whereas the largest contributor for position E312 was distance to ligand. Currently each F_{score} criteria of RMSF, distance to ligand, distance to tryptophan, solvent accessible surface area, and backbone angles are equally weighted. This infers that they will equally influence the change in the fluorophore's environment, however, it is understood that different weightings may be used for enhanced results. Regardless, the present processes have at least a 50% identification rate of labelling positions that have no influence on ligand binding, thus minimizing time and cost of developing efficient, specific, and sensitive biosensors.

In addition to streamlining the identification and selection of non-disruptive labeling positions in a protein of interest, the present processes are robust with regard to fluorophore selection as MalX A128C conjugated with various environmentally sensitive fluorophores was able to report M3 binding. This highlights an additional advantage of the present processes over previously developed labeling approaches, as many such approaches heavily favour a specific protein-fluorophore combination to provide detection of a molecule of interest (e.g. BRUNE, M., et al., Webb, Direct, Real-Time Measurement of Rapid Inorganic Phosphate Release Using a Novel Fluorescent Probe and its Application to Actomyosin Subfragment 1 ATPase. Biochemistry, 33 (1994) 8262-8271; DE LORIMIER, R. M., et al., Construction of a Fluorescent Biosensor Family. Protein Science, 11 (2002) 2655-2675), whereas the present processes have identified an altered environment that can be probed by a wide variety of fluorescent groups. Therefore, our system lends itself to rapid construction of biosensors with user-defined spectroscopic properties and may be easily amenable to development of multiplexed biosensor assays due to the lack of a strong fluorophore preference in biosensors designed and produced using the present processes.

With respect to the developed MOS-detecting biosensor, the fluorophore conjugation site was selected via small-scale changes in local dynamics distal from the MOS binding site (FIG. 10. MalX A128C-MDCC binding of MOS is tight (sub-μM KD), rapid (kₒₙ 20±2 μM⁻¹s⁻¹), and the resulting fluorescence signal change is specific to MOS binding. The kinetic properties of substrate binding for MalX A128C-MDCC are similar to previously developed phosphate-detecting biosensors based on E. coli phosphate binding protein (BRUNE, M., et al., Webb, Direct, Real-Time Measurement of Rapid Inorganic Phosphate Release Using a Novel Fluorescent Probe and its Application to Actomyosin Subfragment 1 ATPase. Biochemistry, 33 (1994) 8262-8271; OKOH, M. P., et al., A Biosensor for Inorganic Phosphate Using a Rhodamine-Labeled Phosphate Binding Protein. Biochemistry, 45 (2006) 14764-14771; SMITH, D. D., et al., Streamlined Purification of Fluorescently Labeled Escherichia Coli Phosphate-Binding Protein (PhoS) Suitable for Rapid-Kinetics Applications. Analytical Biochemistry: Methods in the Biological Sciences, 537 (2017) 106-113), which has enabled detailed kinetic analysis of the timing and role of phosphate release in various systems (KOTHE, U., RODNINA, M. V., Delayed Release of Inorganic Phosphate from Elongation Factor Tu Following GTP Hydrolysis on the Ribosome. Biochemistry, 45 (2006) 12767-12774; PESKE F., et al., Conformationally Restricted Elongation Factor G Retains GTPase Activity but is Inactive in Translocation on the Ribosome. Molecular Cell, 6 (2000) 501-505; RODNINA, M. V., et al., Thiostrepton Inhibits the Turnover but not the GTPase of Elongation Factor G on the Ribosome. Proceedings of the National Academy of Sciences, 96 (1999) 9586-9590; SAVELSBERGH, A., et al., Control of Phosphate Release from Elongation Factor G by Ribosomal Protein L7/12. The EMBO Journal, 24 (2005) 4316-4323). Present methods examine MOS-release from B. licheniformis α-amylase via the present MOS-detecting biosensors. Because the present MOS-detecting biosensors bind MOS rapidly and with high affinity, the observed fluorescence change by the MOS-detecting biosensors are rate-limited by the availability of free MOS in solution produced via starch digestion to MOS. Together, the development of biomolecular tools like the MOS-detecting biosensors will provide an alternative solution to detect and discern between the formation of nascent carbohydrate in real-time and enable rapid characterization of CAZymes, including their enzymatic properties.

The present processes may be used for the development of novel protein-fluorophore conjugates capable of altering their fluorescence state in response to a signal. The present processes may consider, for example, differences in localized altered dynamic properties of each individual amino acid position of a protein in its apo vs. substrate-bound state to direct selection of a site for fluorophore conjugation. The influence of various parameters may be modified in the development of F_{score}, which is disclosed herein as, for example, a scoring algorithm that ranks candidate conjugation positions based on factors that influence fluorescence. The fluorophore conjugation sites are distal to ligand binding surfaces and have no detectable impact on protein function. Altogether, the present processes may be unique compared to conventional approaches as they do not consider large conformational changes of the protein to identify labelling positions, which often impacts protein function and limits scope. The present processes may be used to design and develop biosensors capable of detecting MOS-based on a solute-binding protein with specificity for MOS.

### Example 2

Molecular dynamics simulations of TogB. Structural models of TogB in its apo, digalUA-bound state, unsatdigalUA-bound state, and trigalUA-bound states were obtained from protein databank (2UVG, 2UVH, 2UVI, 2UVJ, respectively (D. W. ABBOTT, A. B. BORASTON, Specific recognition of saturated and 4, 5-unsaturated hexuronate sugars by a periplasmic binding protein involved in pectin catabolism, Journal of molecular biology, 369 (2007) 759-770)). Each model of the protein was solvated using a TIP3P water box extending 10 Å from any point on the protein and the system was neutralized with Na⁺ ions. The potential energy of each molecule was minimized using AMBERFF99S and GLYCAM force fields for 10 000 steps. Subsequently the system was heated to 300 K for 10 000 steps, and molecular dynamics simulations performed for 100 ns at step size of 2 fs using Langevin dynamics to maintain temperature.

The present processes consider small-scale changes in dynamic features of amino acid positions that would have the largest impact on a conjugated fluorophore. Dynamic information comprising backbone flexibility, backbone dihedral angles, distance to ligand, distance to tryptophan residues, and solvent accessibility may be used to develop the F_{score} ranking system. By design, the F_{score} ranking system is dynamic and can weigh changes in the aforementioned criteria differently guided by wet-lab data. As disclosed above, changes in dynamics of backbone dihedral angles between the apo and substrate bound state may alone be used as a metric for producing effective biosensors. The scoring algorithm used in Example 2 relies on changes in backbone dihedral angle dynamics, and data was examined for the apo vs. various different substrate bound states of TogB. Backbone dihedral angles were examined by determining ϕ (between atoms C, N, Cα, and C) and ψ (between atoms N, Cα, C, and N) angles throughout each simulation constructing a Ramachandran plot for each amino acid position. Each Ramachandran plot was transformed into a 180×180 matrix, and the absolute value of the sum of the difference between the ligand-bound (BLr) and apo (BAr) states were determined for each amino acid position, resulting in a single value for each position representing the difference between the apo and substrate-bound state plot. Values were normalized by dividing by the largest difference value in the data set, resulting in F_{score} values ranging from 0-1 (Equation 14). F_{score} values from each replicate were averaged, and averages as well as individual replicates were plotted using GraphPad Prism v. 9.0 (GraphPad Software).

$${F}_{score} = \frac{\sum \vert [ {B}_{L r} ] - [ {B}_{A r} ] \vert}{∨ \sum \vert [ {B}_{L r} ] - [ {B}_{A r} ] \vert} \qquad ( Equation 14 )$$

Construct design. All togB genes were engineered to lack the signal secretion peptide found in wild-type togB as previously described (D. W. ABBOTT, A. B. BORASTON, Specific recognition of saturated and 4, 5-unsaturated hexuronate sugars by a periplasmic binding protein involved in pectin catabolism, Journal of molecular biology, 369 (2007) 759-770). togB variants were synthesized with flanking 5′ NdeI and 3′ XhoI restriction sites and subcloned into pET28a yielding pET28a::togB K94C, pET28a::togB F242C, pET28a::togB A279C, pET28a::togB K357C, and pET28a::togB D358C (BioBasic Canada Inc.). Genes for yePL2b and yeGH28 were subcloned into the 5′ NheI and 3′ XhoI sites of pET28a, yielding pET28a::yePL2b and pET28a::yeGH28.

Overexpression and purification of TogB variants. E. coli BL21(DE3) gold cells transformed with pET28a::togb variants, pET28a::yePL2b, or pET28a::yeGH28 were used to inoculate LB media (10 g/L tryptone, 5 g/L yeast extract, 10 g/L NaCl) supplemented with 50 g/mL kanamycin to an optical density at 600 nm (OD₆₀₀)≈0.1. Cultures were incubated at 37° C. with 200 RPM shaking until OD₆₀₀≈0.6, then incubated at 16° C. with 200 RPM shaking for one hour prior to induction with 300 μM isopropyl-β-D-thiogalactopyranoside (IPTG). Cultures were grown at 16° C. with 200 RPM shaking for 16 hours prior to harvest by centrifugation (5000×g, 15 minutes, 4° C.).

Cells were resuspended in 7 mL of Buffer A (20 mM Tris-Cl (pH 8.0 at 4° C.), 500 mM NaCl, 10 mM imidazole, 10% glycerol, 7 mM β-mercaptoethanol, 1 mM phenylmethylsulfonylfluoride (PMSF) per gram of cells. Lysozyme was added to a final concentration of 1 mg/mL and the cell suspension was incubated on ice for 30 minutes with periodic inversion. Sodium deoxycholate was added at a concentration of 12.5 mg per gram of cells and the cell suspension was mixed on ice for 5 minutes. The mixture was sonicated (Branson Sonifier 450, Danbury, CT, USA) for 30 seconds at 50% output and 50% duty cycle (repeated once, with a 5-minute break between cycles). The mixture was centrifuged (3000×g, 30 minutes, 4° C.) to pellet insoluble cell debris, and the supernatant was centrifuged again (30 000×g, 45 minutes, 4° C.) to collect the S30 fraction. S30 supernatant was loaded onto a 5 mL gravity flow column with Ni²⁺ Sepharose IMAC resin (GE Lifesciences) equilibrated with Buffer A. The column was subsequently washed three times with 3 column-volumes of Buffer A, and four times with 3 column-volumes of Buffer B (Buffer A with 20 mM imidazole) to remove weakly bound proteins. TogB protein variants were eluted in 5 mL fractions using Buffer C (Buffer A with 250 mM imidazole) and examined via Sodium Dodecyl Sulfate Polyacrylamide Gel Electrophoresis (SDS-PAGE) with Coomassie Brilliant Blue staining. Elutions containing TogB variants were pooled and buffer exchanged to Buffer D (20 mM Tris pH 8.0 at 4° C., 30 mM imidazole, 500 mM NaCl, 10% glycerol) using VivaSpin 20 concentrator columns with 10 kDa molecular weight cut off (GE Lifesciences, 15 mL Buffer D added to 5 mL sample, concentrated to 5 mL, repeated 3 times). Purification yields were typically 20-100 mg of protein per liter of culture, and purity was typically >95% based on ImageJ (C. A. SCHNEIDER, W. S. RASBAND, K. W. ELICEIRI, NIH Image to ImageJ: 25 years of image analysis, Nature Methods, 9 (2012) 671) densitometry analysis of Coomassie Brilliant Blue Stained SDS-PAGE.

Overexpression and purification of YePL2b and YeGH28. Overexpression of YePL2b and YeGH28 was done using the same procedure as overexpression of TogB variants, except final concentration of IPTG used for induction was 200 μM (R. MCLEAN, J. K. HOBBS, M. D. SUITS, S. T. TUOMIVAARA, D. R. JONES, A. B. BORASTON, D. W. ABBOTT, Functional analyses of resurrected and contemporary enzymes illuminate an evolutionary path for the emergence of exolysis in polysaccharide lyase family 2, Journal of Biological Chemistry, 290 (2015) 21231-21243). Purification procedure for YePL2b and YeGH28 was the same as the purification of TogB variants except elutions in Buffer C were buffer exchanged into Buffer E (20 mM Tris-HCl (pH 8.0 @ 20° C.)) using dialysis (30 mL sample in 500 mL Buffer E, 4 changes, molecular weight cut off 6-8 kDa). Purification yields were typically 60-100 mg per liter of culture, and purity was typically >95% based on ImageJ (C. A. SCHNEIDER, W. S. RASBAND, K. W. ELICEIRI, NIH Image to ImageJ: 25 years of image analysis, Nature Methods, 9 (2012) 671) densitometry analysis of Coomassie Brilliant Blue Stained SDS-PAGE. Protein concentrations were determined using extinction coefficients ε280, YePL2b=114 835 M⁻¹ cm⁻¹, and ε280, YeGH28=68 425 M⁻¹ cm⁻¹ produced from primary sequence data using ExPASy ProtParam (E. GASTEIGER, C. HOOGLAND, A. GATTIKER, S.e. DUVAUD, M. R. WILKINS, R. D. APPEL, A. BAIROCH, Protein identification and analysis tools on the ExPASy server, Springer2005). Purified proteins were flash frozen and stored at −80° C. for future use.

Fluorescent labeling of TogB variants. TogB variants (100 μM concentration in labeling reaction, 5 mL labeling reaction volume) were each incubated with 2 mL Ni²⁺ Sepharose IMAC resin in Buffer D. 7-Diethylamino-3-[N-(2-maleimidoethyl)carbamoyl]coumarin (MDCC; Sigma PN: 05019; 25 mM stock in dimethylformamide) was added at five-fold molar excess to each TogB variant, corresponding to a 500 μM concentration in the labeling reaction. Labeling reactions were subsequently incubated at 4° C. for 16 hours in an end-over-end mixer. Ni²⁺ Sepharose IMAC resin was collected by centrifugation (500×g, 2 minutes, 4° C.), and supernatant removed. The collected Ni²⁺ Sepharose IMAC resin was washed six times (500×g, 2 minutes, 4° C.) with 3 resin-volumes of Buffer D (500×g, 2 minutes, 4° C.). Bound protein was eluted six times (500×g, 2 minutes, 4° C.) with 1 resin-volume of Buffer F (Buffer D with 250 mM imidizol). Samples from the labeling procedure were examined using SDS-PAGE and the resulting gels imaged (460 nm light, Cy2 Filter, Amersham Imager 600, GE Lifesciences) to confirm the presence of the MDCC label prior to staining with Coomassie Brilliant Blue. Fractions containing the desired protein-fluorophore conjugate were pooled and dialyzed into Buffer G at 4° C. (50 mM Tris-HCl (pH 8.0 @ 20° C.), 500 mM NaCl, 10% glycerol; 4 mL sample into 500 mL Buffer G, 3 changes, molecular weight cut-off 12 kDa). Protein recovery from the labeling procedure was typically ˜50% and labeling efficiencies were typically 60-80%. Concentrations of MDCC-conjugated TogB mutants were determined using spectrophotometry and Equations 15-17. Parameters used were as follows: A280 was the absorbance at 280 nm, A430 was the absorbance at 430 nm, ε280, TogB=90 300 M⁻¹cm⁻¹ is the extinction coefficient of TogB at 280 nm calculated using ExPASy ProtParam based on protein primary sequence (E. GASTEIGER, C. HOOGLAND, A. GATTIKER, S.e. DUVAUD, M. R. WILKINS, R. D. APPEL, A. BAIROCH, Protein identification and analysis tools on the ExPASy server, Springer2005), 0.164 is a correction factor accounting for MDCC absorption at 280 nm (M. BRUNE, J. L. HUNTER, J. E. CORRIE, M. R. WEBB, Direct, real-time measurement of rapid inorganic phosphate release using a novel fluorescent probe and its application to actomyosin subfragment 1 ATPase, Biochemistry, 33 (1994) 8262-8271), L is instrument pathlength in cm, ε430, MDCC=46 800 M⁻¹cm⁻¹ (M. BRUNE, J. L. HUNTER, J. E. CORRIE, M. R. WEBB, Direct, real-time measurement of rapid inorganic phosphate release using a novel fluorescent probe and its application to actomyosin subfragment 1 ATPase, Biochemistry, 33 (1994) 8262-8271). Purified protein-fluorophore conjugates were flash frozen in liquid nitrogen and stored at −80° C. for future use.

$$[ T o g B ] = \frac{{A}_{280 - ( {A}_{430} \times 0.164 )}}{{ε}_{280 , TogB} \times L} \qquad ( Equation 15 )$$

$$MDCC Concentration = \frac{{A}_{430}}{{ε}_{430 , M D C C} \times L} \qquad ( Equation 16 )$$

$$Labeling Efficiency = \frac{Equation 3}{Equation 2} \times 100 \% \qquad ( Equation 17 )$$

Carbohydrates. UnsatdigalUA was produced using methods similar to those described previously (D. W. ABBOTT, A. B. BORASTON, Specific recognition of saturated and 4, 5-unsaturated hexuronate sugars by a periplasmic binding protein involved in pectin catabolism, Journal of molecular biology, 369 (2007) 759-770; D. W. ABBOTT, A. B. BORASTON, A family 2 pectate lyase displays a rare fold and transition metal-assisted (3-elimination, Journal of Biological Chemistry, 282 (2007) 35328-35336). Polygalacturonic acid (PGA, PN:P-PGACT, Megazyme) was dissolved in water at 20 mg/ml and dialyzed into water to remove small carbohydrate impurities (3 500 Da Molecular weight cut off, 50 mL sample into 2 L water, 2 changes). A 50 mL solution of 10 mg/mL PGA was digested overnight at 20° C. with 1 micromolar YePL2b exopolygalacturonate lyase in 1 mM Tris (pH 8.0). The sample solution was evaporated to dryness using a SpeedVac, and re-dissolved in 2 mL water followed by addition of 8 mL of ethanol and 0.5 mL of acetic acid. The tube was then stored at 4° C. for 24 hours, followed by centrifugation (14 000×g, 10 minutes, 20° C.). The pellets were washed with the same acidified ethanol solution and centrifuged (14 000×g, 10 minutes, 20° C.). Supernatants from the two centrifugations were pooled and evaporated to dryness using SpeedVac. Digestions were examined via Thin Layer Chromatography using Silica 60 plates (Millipore) to confirm production of unsatdigalUA (mobile phase 2:1:1 Butanol: acetic acid: water, plates stained in 1% orcinol (PN: 01875, Sigma) in 70:3 ethanol: sulfuric acid, removed from stain and heated using Bunsen burner). Concentrations of unsatdigalUA were determined by mass (FW=352.3 g/mol (D. W. ABBOTT, A. B. BORASTON, Specific recognition of saturated and 4, 5-unsaturated hexuronate sugars by a periplasmic binding protein involved in pectin catabolism, Journal of molecular biology, 369 (2007) 759-770)), and confirmed in solution using spectrophotometry and ε230, unsatdigalUA=5 200 M⁻¹ cm⁻¹ (D. W. ABBOTT, A. B. BORASTON, A family 2 pectate lyase displays a rare fold and transition metal-assisted (3-elimination, Journal of Biological Chemistry, 282 (2007) 35328-35336; V. E. SHEVCHIK, G. CONDEMINE, J. ROBERT-BAUDOUY, N. HUGOUVIEUX-COTTE-PATTAT, The exopolygalacturonate lyase PelW and the oligogalacturonate lyase Ogl, two cytoplasmic enzymes of pectin catabolism in Erwinia chrysanthemi 3937, Journal of bacteriology, 181 (1999) 3912-3919). TrigalUA (PN: T7407) and Galacturonic acid (PN:48280) were purchased from Sigma, and DigalUA (PN: O-GALA2) was purchased from Megazyme.

Equilibrium fluorescence measurements. Fluorescence spectrophotometry measurements were performed using a Quanta Master 60 Fluorescence Spectrometer (Photon Technology International; excitation wavelength 420 nm, emission wavelength 440-520 nm, excitation slit widths: 3 nm, emission slit widths: 6 nm, step size: 1 nm, integration: 1 s). All equilibrium binding measurements were performed in Buffer H (50 mM Tris-HCl (pH 8.0 @ 20° C.), 500 mM NaCl) at 20° C. with MDCC-conjugated TogB variants at a concentration of 100 nM. Carbohydrate concentrations were in at least three-fold excess over the previously reported affinity (K_{D}) values for binding to TogB (D. W. ABBOTT, A. B. BORASTON, Specific recognition of saturated and 4, 5-unsaturated hexuronate sugars by a periplasmic binding protein involved in pectin catabolism, Journal of molecular biology, 369 (2007) 759-770) and were as follows: unsatdigalUA: 16 μM, digalUA: 48 μM, trigalUA: 570 μM. Fluorescence emission spectra were plotted using GraphPad Prism v. 2.0 (GraphPad Software).

Rapidkinetics measurements. A KinTek SF-2004 (Kintek Corp.) rapid mixing device (stopped-flow apparatus) was used for rapid kinetics measurements. Excitation wavelength was 420 nm, and fluorescence emission was detected after passing 450 nm long-pass filters (NewPort Corp.). All experiments in the stopped-flow apparatus were performed in Buffer H. Individual fluorescence time-courses were fit with a one-exponential function (Equation 18), or a two exponential function (Equation 19), where F is the fluorescence observed at time t, F_{∞} is the final fluorescence, A the signal amplitude and, kₐₚₚ the apparent rate (TableCurve, Systat Software). To obtain K_{D} values, a hyperbolic function was fit to the data using GraphPad Prism v. 2.0

$$\begin{matrix}( Equation 20 ) & \\ F = {F}_{∞} + {A}_{1} \times exp ( - {k}_{a p p 1} t ) . & ( Equation 18 )\end{matrix}$$

$$Y = \frac{{B}_{max} \times [ M 3 ]}{{K}_{D} + [ M 3 ]} \qquad ( Equation 19 )$$

$$F = {F}_{∞} + {A}_{1} \times exp ( - {k}_{a p p 1} t ) + {A}_{2} \times exp ( - {k}_{a p p 2} t ) \qquad ( Equation 20 )$$

Use of present processes to select fluorophore conjugation positions in TogB. TogB apo, TogB-unsatdigalUA, TogB-digalUA, and TogB-trigalUA were each subjected to 100 ns molecular dynamics simulations in triplicate and analyzed using the CINC pipeline to determine amino acid F_{score}. Small-scale changes in amino acid dynamics in the apo vs. substrate bound states of TogB were evident when using F_{score}, with several mid to high-scoring positions distal from the ligand binding site (FIG. 11). Conjugation of a fluorescent group distal to the substrate-binding site is preferred as fluorophore conjugation near the binding pocket can lead to defective proteins with altered or reduced substrate specificity (e.g. as in (R. M. DE LORIMIER, J. J. SMITH, M. A. DWYER, L. L. LOOGER, K. M. SALI, C. D. PAAVOLA, S. S. RIZK, S. SADIGOV, D. W. CONRAD, L. LOEW, Construction of a fluorescent biosensor family, Protein Science, 11 (2002) 2655-2675)). To validate the robustness of F_{score} (i.e. for use in cases that may have subtle or drastic changes in amino acid dynamics), a range of mid and high scoring positions were used to construct candidate biosensors labeled with a thiol-reactive fluorophore (FIG. 12; TogB K94C, TogB F242C, TogB A279C, TogB K357C, TogB D358C). The highest scoring position in TogB was position 358 (F_{score}=1), which is located approximately 20 Å from the bound ligand in PDB 2UVI.

Oligogalacturonide detection by candidate biosensors. Each TogB variant was conjugated to MDCC, a diethylaminocoumarin that has been used in the construction of other solute-binding protein-based biosensors (M. BRUNE, J. L. HUNTER, J. E. CORRIE, M. R. WEBB, Direct, real-time measurement of rapid inorganic phosphate release using a novel fluorescent probe and its application to actomyosin subfragment 1 ATPase, Biochemistry, 33 (1994) 8262-8271; J. W. HANES, D. CHATTERJEE, E. V. SORIANO, S. E. EALICK, T. P. BEGLEY, Construction of a thiamin sensor from the periplasmic thiamin binding protein, Chemical Communications, 47 (2011) 2273-2275; D. D. SMITH, D. GIRODAT, H.-J. WIEDEN, L. B. SELINGER, Streamlined purification of fluorescently labeled Escherichia coli phosphate-binding protein (PhoS) suitable for rapid-kinetics applications., Analytical Biochemistry: Methods in the Biological Sciences, 537 (2017) 106-113; S. KUNZELMANN, M. R. WEBB, A biosensor for fluorescent determination of ADP with high time resolution, Journal of Biological Chemistry, 284 (2009) 33130-33138). The MDCC conjugated TogB variants were then examined for their ability to detect unsatdigalUA, digalUA, and trigalUA using fluorescence spectrophotometry. Four of the five TogB-MDCC conjugates were able to detect the target carbohydrates and did not alter their fluorescence in the presence of the non-specific ligand galacturonic acid (Table 4). Therefore, TogB F242C-MDCC, TogB A279C-MDCC, TogB K357C-MDCC, and TogB D358C-MDCC were able to selectively detect the target carbohydrates via a fluorescence change specific to ligand binding. Together, these results demonstrate that examining changes in dihedral angle amino acid dynamics in the present processes alone may be effective at rapidly informing biosensor rational design and streamlining biosensor development.

TABLE 4
Response of Fluorescently Labeled TogB Variants to Ligand

| | Response | | | |
|---|---|---|---|---|
| | w/16 μM | w/48 μM | w/570 μM | w/1710 μM |
| TogB Variant | unsatdigalUA | digalUA | trigalUA | galacturonic acid |
| TogB K94C-MDCC | N.C. | N.C. | N.C. | N.C. |
| TogB F242C-MDCC | −14% | −10% | −20% | N.C. |
| TogB A279C-MDCC | −31% | −31% | −30% | N.C. |
| TogB K357C-MDCC | −32% | −25% | −29% | N.C. |
| TogB D358C-MDCC | −60% | −39% | −44% | N.C. |

Fluorescently labeled TogB variants were incubated in the absence, and presence of saturating concentrations of unsatdigalUA, digalUA, and trigalUA (saturating concentrations defined as ligand concentration at least three-fold above previously reported dissociation constants (D. W. ABBOTT, A. B. BORASTON, Specific recognition of saturated and 4, 5-unsaturated hexuronate sugars by a periplasmic binding protein involved in pectin catabolism, Journal of molecular biology, 369 (2007) 759-770)). Labeled TogB variants were also incubated in the absence, and presence of a non-specific carbohydrate galacturonic acid. Values reported indicate percentage change in peak fluorescence intensity after addition of ligand. N.C. indicates negligible change in fluorescence for a given condition (less than 10% change in fluorescence).

Rapid kinetics of unsatdigalUA and digalUA detection by TogB D358C-MDCC. TogB D358C-MDCC, the highest scorer, displayed the largest fluorescence change in response to the target carbohydrates. For these reasons the rapid kinetic properties of TogB D358C-MDCC were further examined to demonstrate its utility in assays to characterize CAZymes. Kinetic parameters for solute binding to TogB D358C-MDCC were determined using a the stopped-flow method, a rapid mixing device coupled with a fluorescence spectrophotometer enabling real-time monitoring of biomolecular events. In agreement with equilibrium state fluorescence data (vide supra), mixing of TogB D358C-MDCC with increasing concentrations of unsatdigalUA resulted in a fluorescence decrease (FIG. 13A). The resulting fluorescent time-courses were best fit with a single-exponential function to obtain A1 and kₐₚₚ (Equation 18). A1 values of the fluorescence time courses were plotted against concentration of unsatdigalUA and fit with a hyperbolic function to determine K_{D}=1.3±0.5 μM (FIG. 13B, Equation 20). The K_{D} value obtained for unsatdigalUA binding to TogB D358C-MDCC is similar to previously measured affinity values for the unmodified protein determined via isothermal titration calorimetry (K_{D}=5±1 μM) and determined via UV-difference spectroscopy (K_{D}=3.2±0.1 μM) (D. W. ABBOTT, A. B. BORASTON, Specific recognition of saturated and 4, 5-unsaturated hexuronate sugars by a periplasmic binding protein involved in pectin catabolism, Journal of molecular biology, 369 (2007) 759-770). Apparent rate values obtained from fitting fluorescence time-courses were plotted against unsatdigalUA concentration and a linear function fit to the data to determine kₒₙ=18.6±0.7 μM⁻¹s⁻¹ from the slope of the function (FIG. 13C). These results demonstrate TogB D358-MDCC can sensitively and rapidly detect unsatdigalUA, and that fluorophore conjugation did not negatively impact binding affinity for the unsaturated substrate.

The stopped-flow method was again employed to determine kinetic parameters of digalUA binding to TogB D358C-MDCC. TogB D358C-MDCC was rapidly mixed with increasing concentrations of digalUA, and consistent with equilibrium state fluorescence data (vide supra) the resulting time-courses displayed a fluorescence decrease (FIG. 13D). The resulting fluorescent time-courses were best fit with a single-exponential function to obtain A1 and kₐₚₚ (Equation 18). A1 values of the fluorescence time courses were plotted against concentration of digalUA and fit with a hyperbolic function to determine K_{D}=6±1 μM (FIG. 13E, Equation 20). The K_{D} determined for digalUA binding to TogB D358C-MDCC is similar to previously reported binding affinity data determined for the unmodified protein measured via isothermal titration calorimetry (K_{D}=16±2 μM) and measured via UV-difference spectroscopy (K_{D}=11.8±0.5 μM) (D. W. ABBOTT, A. B. BORASTON, Specific recognition of saturated and 4, 5-unsaturated hexuronate sugars by a periplasmic binding protein involved in pectin catabolism, Journal of molecular biology, 369 (2007) 759-770). Apparent rate values obtained from fitting fluorescent time-courses were plotted against digalUA concentration and fit with a linear function to determine kₒₙ=6±1 μM⁻¹s⁻¹ from the slope of the plot (FIG. 13F). Trigal binding to TogB D358C-MDCC was also examined using the stopped-flow method, but under the conditions tested binding likely occurred in the dead-time of the stopped-flow apparatus (data not shown). Together, these results demonstrate that the CINC pipeline is robust in selecting fluorophore conjugation sites that do not disrupt ligand binding, and the resulting TogB D358C-MDCC biosensor is capable of rapid, sensitive substrate detection.

Detection of oligogalacturonide-release from a polysaccharide lyase and a glycoside hydrolase. To demonstrate the utility of TogB D358C-MDCC in characterizing enzyme activity, oligogalacturonide release from CAZyme catalyzed degradation of polygalacturonic acid (PGA) was examined. YePL2b is an exo-acting polysaccharide lyase from Yersinia enterocolitica that degrades PGA to producing unsatdigalUA as the major product (D. W. ABBOTT, A. B. BORASTON, A family 2 pectate lyase displays a rare fold and transition metal-assisted 0-elimination, Journal of Biological Chemistry, 282 (2007) 35328-35336; R. MCLEAN, J. K. HOBBS, M. D. SUITS, S. T. TUOMIVAARA, D. R. JONES, A. B. BORASTON, D. W. ABBOTT, Functional analyses of resurrected and contemporary enzymes illuminate an evolutionary path for the emergence of exolysis in polysaccharide lyase family 2, Journal of Biological Chemistry, 290 (2015) 21231-21243). Using the stopped-flow method, TogB D358C-MDCC (Syringe 1) and YePL2b (Syringe 1) were rapidly mixed with PGA (Syringe 2), and the resulting fluorescent time-courses were best fit with a two-exponential function (FIG. 14A, Equation 19). In the absence of either PGA, or YePL2b, fluorescence output by TogB D358C-MDCC remains unaltered over the time-course of the measurement (FIG. 14A). Fit parameters determined from the aforementioned data show a rapid initial burst phase (kₐₚₚ₁=39±8 s⁻¹) representing the first round of product release. Whereas the second phase (kₐₚₚ₂=0.033±0.005 s⁻¹) represents multiple turnover phase, requiring reorientation of cleaved PGA into the +1 and +2 subsites of YePL2b after each cycle (Table 5). In summary, these results demonstrate that TogB D358C-MDCC can follow unsatdigalUA release from polysaccharide lyase-catalyzed PGA degradation in real-time.

TABLE 5
Oligogalacturonide Release Fit Parameters Obtained
via CAZyme Catalyzed PGA Degradation

| | Oligogalacturonide Release Fit Parameters | | | | |
|---|---|---|---|---|---|
| Standard | A_{inf} (a.u.) | A₁ (a.u.) | k₁ (s⁻¹) | A₂ (a.u.) | k₂ (s⁻¹) |
| YePL2b | 0.545 ± 0.02 | 0.05 ± 0.03 | 39 ± 8 | 0.37 ± 0.03 | 0.033 ± 0.005 |
| YeGH28 | 0.565 ± 0.002 | 0.030 ± 0.004 | 33 ± 1 | 0.300 ± 0.006 | 0.0210 ± 0.0005 |

Fitting of a two-exponential function (Equation 19) to biphasic fluorescence time-courses of oligogalacturonide release shown in FIG. 14 detail underlying enzyme kinetic parameters. Fit parameters for a polysaccharide lyase (YePL2b) and a glycoside hydrolase (YeGH28) are reported (mean ± s.d., n = 6 replicates for each enzyme).

To demonstrate that TogB D358C-MDCC has utility in detecting saturated oligogalacturonide products produced by PGA degradation (i.e. digalUA), product release from YeGH28 was examined. YeGH28 is an exo glycoside hydrolase from Y. entercolitica that degrades PGA producing digalUA as the major product (D. W. ABBOTT, A. B. BORASTON, The structural basis for exopolygalacturonase activity in a family 28 glycoside hydrolase, Journal of molecular biology, 368 (2007) 1215-1222; C.-H. LIAO, L. REVEAR, A. HOTCHKISS, B. SAVARY, Genetic and biochemical characterization of an exopolygalacturonase and a pectate lyase from Yersinia enterocolitica, Canadian journal of microbiology, 45 (1999) 396-403). Using the stopped-flow method, a bi-phasic fluorescence decrease in the presence of TogB D358C-MDCC, YeGH28, and PGA was observed that is not observed in the negative control conditions (FIG. 14B). Fitting the obtained fluorescence time-courses with a two-exponential function showed an initial burst phase (kₐₚₚ₁=33±1 s⁻¹), likely representing the first round of hydrolysis and product release, followed by a slower multiple turnover phase (kₐₚₚ₂=0.0210±0.0005 s⁻¹; Table 5). Together, these findings demonstrate that TogB D358C-MDCC is a robust detection platform for both unsatdigalUA and digalUA and can be used in complex solutions to examine enzyme activity in real-time.

In contrast to the present Example 2, Example 1 used equally weighed dynamics changes in amino acid dihedral angles, RMSF, solvent accessibility, proximity to ligand, and change in distance to tryptophan upon ligand binding as a means to score candidate labeling positions in silico. Example 2, however, demonstrates construction of multiple biosensors for the detection of unsatdigalUA, digalUA, and trigalUA using TogB via a modified process that considered a different number of sets of dynamic information. Of the five mid and high-scoring candidates tested, four resulted in biosensors capable of detecting the target molecules. With the success rate of this embodiment at 80%, it outperforms prior approaches of selecting fluorescent labeling positions based on structural data (e.g. R. M. DE LORIMIER, J. J. SMITH, M. A. DWYER, L. L. LOOGER, K. M. SALI, C. D. PAAVOLA, S. S. RIZK, S. SADIGOV, D. W. CONRAD, L. LOEW, Construction of a fluorescent biosensor family, Protein Science, 11 (2002) 2655-2675). It also experimentally demonstrates the importance of changes in dihedral angle dynamics in some biomolecules compared to other sets of dynamic information.

It is further demonstrated that the biosensors produced by this pipeline are robust for in vitro detection of oligogalacturonides. TogB D358C-MDCC was engineered based on small-scale changes in amino acid dynamics distal from the ligand binding site, and the engineered fluorescent protein conjugate has comparable ligand binding affinity to the unmodified protein (vide infera). The present embodiment has enabled detailed kinetic analysis of CAZymes that release maltooligoaccharides as part of their functional cycle, which was verified by comparing unsatdigalUA release by a polysaccharide lyase (YePL2b) and digalUA released by a glycoside hydrolase (YeGH28) during PGA degradation via TogB D358C-MDCC. TogB D358C-MDCC binds unsatdigalUA and digalUA rapidly and with high affinity, which means that that the observed fluorescence change by TogB D358C-MDCC is rate-limited by the availability of free oligogalacturonides in solution produced via CAZyme catalyzed PGA degradation. TogB D358C-MDCC will enable an alternative solution to rapid characterization of CAZymes, as well as providing the additional capability of detecting and distinguishing between the formation of a nascent carbohydrate and its release into bulk solution. The present processes illustrated by this Example 2 will allow rapid biosensor generation and provide a transformative solution to the traditionally laborious process of biosensor development.

### Example 3

Molecular dynamics simulations of EF-Tu. Structural model of Escherichia coli EF-Tu in GDPNP-bound state was derived from homology modelling of the Thermus aquaticus EF-Tu obtained from the Protein Data Bank archive (lEFT). In this model GDPNP was manually converted to GTP. The GDP-bound conformation of Escherichia coli EF-Tu was obtained directly from the Protein Data Bank archive (1EFC). Hydrogen was added to the system with the psfgen package within the NAMD software (1). Each model of the protein was solvated with a TIP3P water box protruding 10 Å from any point on the protein using the Visual Molecular Dynamics (VMD) software. The potential energy of the systems was minimized using a two-fold iterative process minimizing the potential energy of water molecules followed by a minimization of the protein using CHARMM 27 parameters for 10 000 steps each using the NAMD software. Each EF-Tu conformation was subsequently neutralized with NaCl ions followed by a final minimization of the entire system for 100 000 steps. EF-Tu systems were then equilibrated to 300 K and 350 K for 300 000 steps. Finally, MD simulations were performed at 300 K using velocities for the 350 K equilibration and atomic positions from the 300 K equilibration for 100 ns at a step size of 2 fs using Langevin dynamics to maintain temperature using the NAMD software.

F_{score} for EF-Tu E202C-Dansyl. Dynamic information comprising backbone flexibility, backbone dihedral angles, solvent accessibility, and distance to ligand were measured to develop a ranking score (FIG. 1). These properties were chosen as small dynamic features of the amino acid position that would have the largest impact on the environment of the conjugated fluorophore. Flexibility was determined by measuring the Root Mean Square Fluctuation (RMSF) of the Cα atoms of each amino acid over the course of both the ligand bound (FLr) and apo (FAr) simulations (Equation 21). All parameters were measured using the VMD software.

$${F}_{score} = \frac{\vert 〈 {F}_{L r} 〉 - 〈 {F}_{A r} 〉 \vert}{∨ \vert 〈 {F}_{L r} 〉 - 〈 {F}_{A r} 〉 \vert} \qquad ( Equation 21 )$$

F_{score} for EF-Tu L265C-Dansyl. Dynamic information comprising backbone flexibility, backbone dihedral angles, solvent accessibility, distance to ligand, and change in distance (GDP-bound vs. GTP-bound) between each amino acid position and the sole tryptophan (W185) residue were measured to develop a ranking score (FIG. 1). The last set of dynamic information was chosen as distance between reporter group and tryptophan residues is one of the major factors effecting FRET. This consideration allowed a suitable position to be selected for modification by a fluorescent reporter group that could report distance changes between the native tryptophan and the reporter group in a GDP- vs. GTP-bound state.

F_{score} for EF-Tu T34C L265C-IAEDANS/DDPM. Dynamic information comprising backbone flexibility, backbone dihedral angles, solvent accessibility, distance to ligand, change in distance (GDP-bound vs. GTP-bound) between each amino acid position and the sole tryptophan (W185) residue, and change in distance (GDP-bound vs. GTP-bound) between each two amino acid positions were measured to develop a ranking score (FIG. 1). The last set of dynamic information was chosen as distance between amino acid positions is one of the major factors effecting FRET. This allowed us to select a position to label with two unique fluorescent reporter groups that could report distance changes in a GDP- vs. GTP-bound state.

Biosensor construct design. Criteria for selection of these mutants was guided by the CINC pipeline and is explained in detail in the Results section (vide infra). All constructs encoded a C-terminal poly-Histidine tag fusion.

Expression of EF-Tu variants. Escherichia coli BL21(DE3) gold cells (Agilent) transformed with either pET21a::tufA T34C L265C, pET21a::tufA L265C, or p pET21a::tufA E202C were used to inoculate LB media (10 g/L typtone, 5 g/L yeast extract, 10 g/L NaCl) supplemented with 50 mg/L kanamycin to an optical density at 600 nm (OD₆₀₀)≈0.1. After growth at 37° C. with 200 RPM shaking, isopropyl β-D-1-thiogalactopyranoside (IPTG) was added to a final concentration of 1 mM when OD₆₀₀ reached approximately 0.6. Cultures were grown for an additional 3 hours, cells harvested by centrifugation (5000×g, 10 minutes, 4° C.), flash frozen, and stored at −80° C. for further use.

Purification of EF-Tu variants. Cell pellets were resuspended in 7 mL/g of Buffer 1 (50 mM Tris Cl 8.0 (4° C.), 60 mM NH₄Cl, 7 mM MgCl₂, 7 mM β-mercaptoethanol, 1 mM PMSF, 300 mM KCl, 10 mM Imidazol, 15% glycerol, 50 μM GDP), and lysozyme added to a final concentration of 1 mg/mL prior to a 30-minute incubation at 4° C. Sodium deoxycholate was added to a final concentration of 12.5 mg per gram of cells, and the suspension was sonicated (Branson Sonifier 450) on ice for 5 min at 50% output and 60% duty cycle). The mixture was centrifuged (3000×g, 30 minutes, 4° C.) to pellet insoluble debris, followed by additional centrifugation to produce S30 supernatant (30 000×g, 45 minutes, 4° C.). S30 supernatant was applied to Ni²⁺-Sepharose resin in a batch-chromatography setup (3 mL resin per 1 g of cells opened) and incubated 30 minutes at 4° C. Ni²⁺-Sepharose resin was collected by centrifugation (500×g, 2 minutes, 4° C.), and supernatant decanted. The collected Ni²⁺-Sepharose resin was washed 3 times with 10 resin-volumes of Buffer 1, followed by 4 washes with 10 resin-volumes Buffer 2 (Buffer 1 with 20 mM imidazole). Bound protein was eluted six times with 1 resin-volume Buffer 3 (Buffer 1 with 250 mM imidazole). Samples were analyzed via sodium dodecyl sulfate polyacrylamide gel electrophoresis (SDS-PAGE) stained with Coomassie Brilliant Blue, and elutions containing each EF-Tu variant were pooled and concentrated to ˜5 mL using a Vivaspin protein concentrator spin column (Cytiva Life Sciences). Concentrated EF-Tu was then loaded onto a Superdex 75 size gel filtration column equilibrated with TAKM7 buffer (50 mM Tris Cl 7.5 (40C), 70 mM NH₄Cl, 30 mM KCl, 7 mM MgCl₂). Samples were analyzed via sodium dodecyl sulfate polyacrylamide gel electrophoresis (SDS-PAGE) stained with Coomassie Brilliant Blue, and elutions containing each EF-Tu variant were pooled. Purified protein was flash-frozen with liquid nitrogen and stored at −80° C. for further use.

Fluorescent labeling of EF-Tu variants. 100,000 pmol of EF-Tu was thawed on ice and diluted 5-fold (˜12 μM final concentration) in Buffer F (25 mM Tris-Cl pH 7.5 (4° C.), 7 mM MgCl₂, 30 mM KCl, and 20% (v/v) glycerol) before adding a 10-fold molar excess of either a single dye (N-92-(Dansylamino)ethyl)maleimide (Dansyl)) or an equimolar mixture of two dyes 5-(2-iodoacetylaminoethyl)aminonaphthalene-1-sulfonic acid (IAEDANS) and N-(4-dimethylamino-3,5-dinitrophenyl)maleimide (DDPM), dropwise on ice. The sample was then incubated at 4° C. for 4 hours or overnight with constant inversion before being centrifuged at 21000 g for 5 min to pellet any precipitate. The labelled protein was then separated from excess dye by size exclusion chromatography (XK16/20 column; Superdex-G25 (GE Healthcare)). Fractions containing labeled EF-Tu were identified by SDS PAGE, pooled and flash frozen in liquid nitrogen. Protein recovery for labeling was often greater than 80%.

Equilibrium fluorescence experiments. Fluorescence spectrophotometry was performed using a Quanta Master 60 Fluorescence Spectrometer (Photon Technology International, all experiments utilized 1 nm step size, 1 s integration). For all experiments labelled EF-Tu binary complexes were formed by incubating EF-Tu with a 100-fold molar excess of either GTP or GDP at 37° C. for 20 min. Complexes were then excited at either 280 nm (EF-Tu-L265C-Dansyl), 560 nm (EF-Tu T34C L265C-IAEDANS/DDPM), or 335 nm (EF-Tu E202C-Dansyl) and fluorescent emission was detected via fluorescence spectrophotometer (PTI).

Rapid-kinetics measurements. Rapid kinetics experiments were performed in a KinTek SF-2004 stopped-flow apparatus (KinTek Corp.) at 20° C. An excitation wavelength of 280 nm or 335 nm was used for all rapid kinetics experiments, and fluorescence emission was measured through a 350 nm long-pass filter.

Nucleotide dissociation rate constants were determined as previously described (E. I. DE LAURENTIIS, F. MO, H. J. WIEDEN, Construction of a fully active Cys-less elongation factor Tu: functional role of conserved cysteine 81, Biochimica et biophysica acta 1814(5) (2011) 684-92.). No mutants with altered nucleotide affinities were selected for future characterization (Table 6, FIG. 24, and FIG. 25). EF-Tu was incubated with a 10-fold molar excess of 2′-(or-3′)-O—(N-Methylanthraniloyl) Guanosine 5′-Triphosphate (mantGTP, Thermo Fisher Scientific) or 2′-(or-3′)-O—(N-Methylanthraniloyl) Guanosine 5′-Diphosphate (mantGDP, Thermo Fisher Scientific) in TAKM7 at 37° C. for 20 min to form EF-Tu·mantGDP and EF-Tu·mantGTP binary complexes respectively. Reactions containing mantGTP also contained 3 mM phosphoenol pyruvate (PEP) and 20 U/mL pyruvate kinase (Roche Diagnostic) to ensure all nucleotides were in triphosphate form. 25 μL of EF-Tu·mantGTP/mantGDP (0.15 μM after mixing) was rapidly mixed with 25 μL of excess GTP/GDP at 20° C. in TAKM7. Mant-nucleotides were excited via FRET from the tryptophan residue present in EF-Tu (Xex=280 nm) and emitted fluorescence passed through a LG-400F cut off filter (NewPort) before detection. Individual fluorescence time-courses were fit with a one exponential function (Eq. 12), where F is fluorescence observed at time t, F_{∞} is final fluorescence, A is signal amplitude, and kₐₚₚ is apparent rate (TableCurve, Systat Software).

Conformational changes were measured by mixing EF-Tu (either EF-Tu L265C-Dansyl or EF-Tu E202C-Dansyl) against 10 mM ethylenediaminetetraacetic acid. 25 μL of EF-Tu·GDP or EF-Tu·GTP (0.3 μM after mixing, prepared as described above) with 25 μL with of EDTA (10 mM after mixing). Dansyl was excited either by FRET (as above) or directly at 335 nm and emitted fluorescence passed through a LG-350F cut off filter (NewPort) before detection. Individual fluorescence time-courses were fit with a one exponential function (Eq. 12), where F is fluorescence observed at time t, F_{∞} is final fluorescence, A is signal amplitude, and kₐₚₚ is apparent rate (TableCurve, Systat Software).

Hydrolysis Protection Assay. EF-Tu·GTP·[¹⁴C]Phe-tRNAPhe ternary complexes were formed as described previously and incubated at 37° C. Aliquots were removed at various time points (0-100 min) and the amount of [¹⁴C]Phe was measured using a Tri-Carb 2800TR Perkin Elmer Liquid Scintillation Analyzer and data plotted as described previously (E. I. DE LAURENTIIS, F. MO, H. J. WIEDEN, Construction of a fully active Cys-less elongation factor Tu: functional role of conserved cysteine 81, Biochimica et biophysica acta 1814(5) (2011) 684-92.). The fluorescent dye 5-(2-iodoacetylaminoethyl)aminonaphthalene-1-sulfonic acid (IAEDANS, λex=336 nm, λem=454 nm) and the fluorescent quencher N-(4-dimethylamino-3,5-dinitrophenyl)maleimide (DDPM) were selected as they have a small reported R0 (˜27 Å). Upon the excitation of EF-Tu T34C L265C-IAEDANS/DDPM·GDP at 336 nm peak fluorescent emission at ˜473 nm were observed (FIG. 16) and ˜4× more IAEDANS fluorescence was repeatedly detected when EF-Tu T34C L265C-IAEDANS/DDPM was bound to GTP (FIG. 16) although according to the crystal structures the two positions should be ˜22 Å closer (FIG. 15). This was confirmed to not be due to a difference in EF-Tu concentration and indeed a difference in FRET by trypsin digestion (FIG. 17). Although a single gaussian peak is seen for the trypsin digested EF-Tu T34C L265C-IAEDANS/DDPM there are multiple populations observed in both and GTP bound conformations (FIG. 16).

In order to deconvolute signals, a single reporter group embodiment was used, wherein the native tryptophan was used as the donor fluorophore and using dansyl as the acceptor to measure intramolecular heteroFRET with only a single fluorescent dye. Present processes were used to select a position to introduce a cystine into a cysless EF-Tu and L265C was identified as a suitable candidate. Upon excitation of EF-Tu L265C-Dansyl·GDP at 280 nm a large fluorescence emission was observed for tryptophan (max ˜325 nm) and no dansyl fluorescence (FIG. 18) indicating no FRET is occurring in this state. Conversely when EF-Tu L265C-Dansyl·GTP is excited at 280 nm there is a distinct dansyl peak (max ˜440 nm) that is observed in addition to the aforementioned tryptophan peak (FIG. 18).

The present embodiment accurately reports conformational differences in EF-Tu. It may further be used to measure conformational changes over time, specifically as they relate to nucleotide dissociation. Methods disclosed herein were used to get real time measurements of EF-Tus conformational changes as it transitions from the nucleotide bound to apo state.

As nucleotide dissociation is naturally very slow a mixture of EF-Tu bound to GDP or GTP against EDTA was used to chelate EF-Tu associated magnesium (which coordinates the nucleotide phosphates) accelerating nucleotide dissociation. When EF-Tu L265C-Dansyl·GDP was mixed with EDTA there was no change in fluorescence, although nucleotide dissociation was occurring (FIG. 19). EF-Tu L265C-Dansyl·GTP however, in addition to staring at a higher initial fluorescence (consistent with steady state measurements, FIG. 18) undergoes a slow increase in fluorescence (FIG. 19) upon mixing with EDTA, with an apparent rate of 0.05±0.02 s⁻¹. This is an order of magnitude slower than nucleotide dissociation under these conditions (GTP and GDP are dissociating at the same rate (0.35±0.19s⁻¹ and 0.45±0.26s⁻¹ respectively)) and is identical to the native rate of GTP dissociation from EF-Tu suggesting that the change in conformation may limit nucleotide dissociation. Directly exciting the acceptor dye (dansyl) rather that tryptophan resulted in no change in fluorescence for both GTP and GDP bound EF-Tu (FIG. 20) demonstrating this was indeed due to FRET.

While changes for EF-Tu L265C-Dansyl·GTP were visualized, it was also desirable to find a labelled EF-Tu that could report conformational changes for both nucleotides bound states over time. Therefore, the present processes were used to identified several candidates based on local dye environment rather than a FRET based approach from this E202C was selected for further characterization.

Upon direct excitation at 335 nm a fluorescence emission can be observed for EF-Tu E202C-Dansyl·GDP, however, when bound to GTP there is a distinct increase in fluorescence (˜1.5×) in addition to a large blue shift in the maxima (GDP ˜500 nm, GTP ˜475 nm) (FIG. 21). As with EF-Tu L265C-Dansyl the fluorescence peaks from EF-Tu E202C-Dansyl indicated multiple populations for both nucleotide bound conformations. Upon mixing with EDTA, EF-Tu E202C-Dansyl·GTP undergoes a conformational change to a higher fluorescent state with an identical rate to that measured for EF-Tu L265C-Dansyl·GTP, demonstrating the consistency of our system. While EF-Tu L265C-Dansyl·GDP did not have a change in fluorescence upon mixing with EDTA, EF-Tu E202C-Dansyl·GDP undergoes a large change in fluorescence (˜15%, compared to 5% for EF-Tu E202C-Dansyl·GTP, FIG. 22). This conformational change is an order of magnitude slower than two previously measured GTP rates (0.0016±0.002s⁻¹).

TABLE 6
Nucleotide Dissociation Rates from EF-Tu Variants

Nucleotide Dissociation Rates

| | GTP Dissociation | GDP Dissociation |
|---|---|---|
| EF-Tu Variant | Rate k_{off} (s⁻¹) | Rate K_{off} (s⁻¹) |
| Unmodified EF-Tu | 0.04 ± 0.01 | 0.002 ± 0.001 |
| EF-Tu T34C L265C | 0.02 ± 0.01 | 0.002 ± 0.001 |
| EF-Tu T34C L265C - | 0.014 ± 0.002 | 0.003 ± 0.001 |
| IAEDANS/DDPM | | |
| EF-Tu E202C-Dansyl | 0.015 ± 0.005 | 0.05 0.001 |

These EF-Tu biosensors display several strengths of the system, it shows this extends beyond carbohydrate binding proteins and can generate biosensors that are capable to reporting in multiple ways which small molecule is bound. E202C-Dansyl reports changes in dye environment much like the MalX and TogB biosensors but shows CINC is amenable to small molecule binders and different protein classes. L265C-Dansyl uses FRET to report the change in distance between the native tryptophan and Dansyl. T34C L265C also uses FRET but between two fluorescent dyes as opposed to using a single non-biological dye.

### SUMMARY

Disclosed herein are processes for designing and producing a modified biomolecule, wherein the processes comprise the steps of: selecting at least one biomolecule suitable for modification; obtaining at least one structure of the at least one biomolecule; simulating the molecular dynamics of the at least one structure to generate dynamic information about at least one position within the at least one structure; using the dynamic information to calculate a score for the at least one position; comparing the score with at least one reference score to identify at least one target position within the biomolecule suitable for modification; and modifying the at least one target position.

Further disclosed herein is an embodiment of the said processes wherein the at least one biomolecule comprises a polypeptide.

Further disclosed herein is an embodiment of the said processes wherein the at least one biomolecule comprises a nucleic acid.

Further disclosed herein is an embodiment of the said processes wherein the at least one biomolecule comprises a lipid.

Further disclosed herein is an embodiment of the said processes wherein the at least one biomolecule comprises a carbohydrate.

Further disclosed herein is an embodiment of the said processes wherein the modifying of the at least one target position comprises the addition of a reporter group.

Further disclosed herein is an embodiment of the said processes wherein the reporter group comprises a redox cofactor.

Further disclosed herein is an embodiment of the said processes wherein the reporter group comprises a fluorophore.

Further disclosed herein is an embodiment of the said processes wherein the modifying of the at least one target position comprises the addition of a linker.

Further disclosed herein is an embodiment of the said processes wherein the modifying of the at least one target position comprises an intramolecular modification selected from the group consisting of at least one addition, at least one deletion, and at least one substitution.

Further disclosed herein is an embodiment of the said processes wherein the intramolecular modification results in the introduction of at least one cysteine residue.

Further disclosed herein is an embodiment of the said processes wherein the at least one structure comprises a three-dimensional representation of the at least one biomolecule in an apo configuration.

Further disclosed herein is an embodiment of the said processes wherein the at least one structure comprises a three-dimensional representation of the at least one biomolecule in a ligand-bound configuration.

Further disclosed herein is an embodiment of the said processes wherein obtaining the at least one structure is by a method selected from the group consisting of crystallography, cryogenic electron microscopy (cryo-EM), nuclear magnetic resonance (NMR) spectroscopy, or electron paramagnetic resonance (EPR) spectroscopy.

Further disclosed herein is an embodiment of the said processes wherein obtaining the at least one structure is by a method comprising prediction modelling.

Further disclosed herein is an embodiment of the said processes wherein the score for the at least one position is compared to a reference score for at least one other position within the at least one structure.

Further disclosed herein is an embodiment of the said processes wherein the score for the at least one position is compared to a reference score that is pre-determined.

Further disclosed herein is an embodiment of the modified biomolecule when designed and produced by the said processes, wherein the modified biomolecule is a biosensor for maltooligosaccharides that comprises a Streptococcus pneumoniae (S. pneumoniae) MalX polypeptide and at least one reporter group.

Further disclosed herein is an embodiment of the said biosensor when designed and produced by the said processes wherein the biosensor is for maltooligosaccharides having a degree of polymerization of between three to eleven glucose residues.

Further disclosed herein is an embodiment of the said biosensor when designed and produced by the said processes wherein the at least one reporter group is attached at one or more amino acid positions of the S. pneumoniae MalX polypeptide.

Further disclosed herein is an embodiment of the said biosensor when designed and produced by the said processes wherein the at least one reporter group is attached at amino acid position 128 or 243 of the S. pneumoniae MalX polypeptide.

Further disclosed herein is an embodiment of the said biosensor when designed and produced by the said processes wherein the at least one reporter group is covalently attached at amino acid position 128 or 243 of the S. pneumoniae MalX polypeptide.

Further disclosed herein is an embodiment of the said biosensor when designed and produced by the said processes wherein the at least one reporter group is noncovalently attached at amino acid position 128 or 243 of the S. pneumoniae MalX polypeptide.

Further disclosed herein is an embodiment of the said biosensor when designed and produced by the said processes wherein the S. pneumoniae MalX polypeptide is a A128C or T243C variant.

Further disclosed herein is an embodiment of the said biosensor when designed and produced by the said processes wherein the modified biomolecule is a biosensor for homogalacturonan breakdown products that comprises a Yersinia enterocolitica (Y. enterocolitica) TogB polypeptide and at least one reporter group.

Further disclosed herein is an embodiment of the said biosensor when designed and produced by the said processes for homogalacturonan breakdown products selected from the group consisting of 4,5-unsaturated digalacturonic acid, digalacturonic acid, and trigalacturonic acid.

Further disclosed herein is an embodiment of the said biosensor when designed and produced by the said processes wherein the at least one reporter group is attached at one or more amino acid positions of the Y. enterocolitica TogB polypeptide.

Further disclosed herein is an embodiment of the said biosensor when designed and produced by the said processes wherein the at least one reporter group is attached at an amino acid position selected from the group consisting of 242, 279, 357, and 358 of the Y. enterocolitica TogB polypeptide.

Further disclosed herein is an embodiment of the said biosensor when designed and produced by the said processes wherein the at least one reporter group is covalently attached at an amino acid position selected from the group consisting of 242, 279, 357, and 358 of the Y. enterocolitica TogB polypeptide.

Further disclosed herein is an embodiment of the said biosensor when designed and produced by the said processes wherein the at least one reporter group is noncovalently attached at an amino acid position selected from the group consisting of 242, 279, 357, and 358 of the Y. enterocolitica TogB polypeptide.

Further disclosed herein is an embodiment of the said biosensor when designed and produced by the said processes wherein the Y. enterocolitica TogB polypeptide is a variant selected from the group consisting of F242C, A279C, K357C, and D358C.

Further disclosed herein is an embodiment of the said biosensor when designed and produced by the said processes wherein the modified biomolecule is a biosensor for observing conformational changes that comprises an Escherichia coli (E. coli) EF-Tu polypeptide and at least one reporter group.

Further disclosed herein is an embodiment of the said biosensor when designed and produced by the said processes wherein the at least one reporter group is attached at one or more amino acid positions of the E. coli EF-Tu polypeptide.

Further disclosed herein is an embodiment of the said biosensor when designed and produced by the said processes wherein the at least one reporter group is attached at amino acid position 202 or 265 of the E. coli EF-Tu polypeptide.

Further disclosed herein is an embodiment of the said biosensor when designed and produced by the said processes wherein the at least one reporter group is covalently attached at amino acid position 202 or 265 of the E. coli EF-Tu polypeptide.

Further disclosed herein is an embodiment of the said biosensor when designed and produced by the said processes wherein the at least one reporter group is noncovalently attached at amino acid position 202 or 265 of the E. coli EF-Tu polypeptide.

Further disclosed herein is an embodiment of the said biosensor when designed and produced by the said processes wherein the E. coli EF-Tu polypeptide is a T34C E202C or T34C L265C variant.

Further disclosed herein is an embodiment of the said biosensor when designed and produced by the said processes wherein the reporter group comprises a redox cofactor.

Further disclosed herein is an embodiment of the said biosensor when designed and produced by the said processes wherein the reporter group comprises a fluorophore.

Further disclosed herein is an embodiment of the said biosensor when designed and produced by the said processes wherein the fluorophore is from the group consisting of a member of the naphthalene family, a member of the xanthene family, and a member of the pyrene family.

Further disclosed herein is an embodiment of the said biosensor when designed and produced by the said processes wherein the fluorophore is from the group consisting of 7-diethylamino-3-(4′-maleimidylphenyl)-4-methylcoumarin (CPM), 7-diethylamino-3-[N-(2-maleimidoethyl)carbamoyl]coumarin (MDCC), N-(7-dimethylamino-4-methylcoumarin-3-yl)maleimide (DACM), N-[2-(dansylamino)ethyl]maleimide (Dansyl), fluorescein-5-maleimide (Fluorescein), N-(1-pyrene)maleimide (Pyrene), Rhodamine Red C2 maleimide (Rhodamine Red), and 5-(2-iodoacetylaminoethyl)aminonaphthalene-1-sulfonic acid (IAEDANS).

Disclosed herein is a biosensor for maltooligosaccharides that comprises a Streptococcus pneumoniae (S. pneumoniae) MalX polypeptide and at least one reporter group.

Further disclosed herein is an embodiment of the said biosensor for maltooligosaccharides having a degree of polymerization of between three to eleven glucose residues.

Further disclosed herein is an embodiment of the said biosensor wherein the at least one reporter group is attached at one or more amino acid positions of the S. pneumoniae MalX polypeptide.

Further disclosed herein is an embodiment of the said biosensor wherein the at least one reporter group is attached at amino acid position 128 or 243 of the S. pneumoniae MalX polypeptide.

Further disclosed herein is an embodiment of the said biosensor wherein the at least one reporter group is covalently attached at amino acid position 128 or 243 of the S. pneumoniae MalX polypeptide.

Further disclosed herein is an embodiment of the said biosensor wherein the at least one reporter group is noncovalently attached at amino acid position 128 or 243 of the S. pneumoniae MalX polypeptide.

Further disclosed herein is an embodiment of the said biosensor wherein the S. pneumoniae MalX polypeptide is a A128C or T243C variant.

Further disclosed herein is an embodiment of the said biosensor wherein the reporter group comprises a redox cofactor.

Further disclosed herein is an embodiment of the said biosensor wherein the reporter group comprises a fluorophore.

Further disclosed herein is an embodiment of the said biosensor wherein the fluorophore is from the group consisting of a member of the naphthalene family, a member of the xanthene family, and a member of the pyrene family.

Further disclosed herein is an embodiment of the said biosensor wherein the fluorophore is from the group consisting of 7-diethylamino-3-(4′-maleimidylphenyl)-4-methylcoumarin (CPM), 7-diethylamino-3-[N-(2-maleimidoethyl)carbamoyl]coumarin (MDCC), N-(7-dimethylamino-4-methylcoumarin-3-yl)maleimide (DACM), N-[2-(dansylamino)ethyl]maleimide (Dansyl), fluorescein-5-maleimide (Fluorescein), N-(1-pyrene)maleimide (Pyrene), Rhodamine Red C2 maleimide (Rhodamine Red), and 5-(2-iodoacetylaminoethyl)aminonaphthalene-1-sulfonic acid (IAEDANS).

Disclosed herein is a biosensor for homogalacturonan breakdown products that comprises a Yersinia enterocolitica (Y. enterocolitica) TogB polypeptide and at least one reporter group.

Further disclosed herein is an embodiment of the said biosensor for homogalacturonan breakdown products selected from the group consisting of 4,5-unsaturated digalacturonic acid, digalacturonic acid, and trigalacturonic acid.

Further disclosed herein is an embodiment of the said biosensor wherein the at least one reporter group is attached at one or more amino acid positions of the Y. enterocolitica TogB polypeptide.

Further disclosed herein is an embodiment of the said biosensor wherein the at least one reporter group is attached at an amino acid position selected from the group consisting of 242, 279, 357, and 358 of the Y. enterocolitica TogB polypeptide.

Further disclosed herein is an embodiment of the said biosensor wherein the at least one reporter group is covalently attached at an amino acid position selected from the group consisting of 242, 279, 357, and 358 of the Y. enterocolitica TogB polypeptide.

Further disclosed herein is an embodiment of the said biosensor wherein the at least one reporter group is noncovalently attached at an amino acid position selected from the group consisting of 242, 279, 357, and 358 of the Y. enterocolitica TogB polypeptide.

Further disclosed herein is an embodiment of the said biosensor wherein the Y. enterocolitica TogB polypeptide is a variant selected from the group consisting of F242C, A279C, K357C, and D358C.

Further disclosed herein is an embodiment of the said biosensor wherein the reporter group comprises a redox cofactor.

Further disclosed herein is an embodiment of the said biosensor wherein the reporter group comprises a fluorophore.

Further disclosed herein is an embodiment of the said biosensor wherein the fluorophore is from the group consisting of a member of the naphthalene family, a member of the xanthene family, and a member of the pyrene family.

Further disclosed herein is an embodiment of the said biosensor wherein the fluorophore is from the group consisting of 7-diethylamino-3-(4′-maleimidylphenyl)-4-methylcoumarin (CPM), 7-diethylamino-3-[N-(2-maleimidoethyl)carbamoyl]coumarin (MDCC), N-(7-dimethylamino-4-methylcoumarin-3-yl)maleimide (DACM), N-[2-(dansylamino)ethyl]maleimide (Dansyl), fluorescein-5-maleimide (Fluorescein), N-(1-pyrene)maleimide (Pyrene), Rhodamine Red C2 maleimide (Rhodamine Red), and 5-(2-iodoacetylaminoethyl)aminonaphthalene-1-sulfonic acid (IAEDANS).

Disclosed herein is a biosensor for observing conformational changes that comprises an Escherichia coli (E. coli) EF-Tu polypeptide and at least one reporter group.

Further disclosed herein is an embodiment of the said biosensor wherein the at least one reporter group is attached at one or more amino acid positions of the E. coli EF-Tu polypeptide.

Further disclosed herein is an embodiment of the said biosensor wherein the at least one reporter group is attached at amino acid position 202 or 265 of the E. coli EF-Tu polypeptide.

Further disclosed herein is an embodiment of the said biosensor wherein the at least one reporter group is covalently attached at amino acid position 202 or 265 of the E. coli EF-Tu polypeptide.

Further disclosed herein is an embodiment of the said biosensor wherein the at least one reporter group is noncovalently attached at amino acid position 202 or 265 of the E. coli EF-Tu polypeptide.

Further disclosed herein is an embodiment of the said biosensor wherein the E. coli EF-Tu polypeptide is a T34C E202C or T34C L265C variant.

Further disclosed herein is an embodiment of the said biosensor wherein the reporter group comprises a redox cofactor.

Further disclosed herein is an embodiment of the said biosensor wherein the reporter group comprises a fluorophore.

Further disclosed herein is an embodiment of the said biosensor wherein the fluorophore is from the group consisting of a member of the naphthalene family, a member of the xanthene family, and a member of the pyrene family.

Further disclosed herein is an embodiment of the said biosensor wherein the fluorophore is from the group consisting of 7-diethylamino-3-(4′-maleimidylphenyl)-4-methylcoumarin (CPM), 7-diethylamino-3-[N-(2-maleimidoethyl)carbamoyl]coumarin (MDCC), N-(7-dimethylamino-4-methylcoumarin-3-yl)maleimide (DACM), N-[2-(dansylamino)ethyl]maleimide (Dansyl), fluorescein-5-maleimide (Fluorescein), N-(1-pyrene)maleimide (Pyrene), Rhodamine Red C2 maleimide (Rhodamine Red), and 5-(2-iodoacetylaminoethyl)aminonaphthalene-1-sulfonic acid (IAEDANS).

1. A process for designing and producing a modified biomolecule, wherein the process comprises:
selecting at least one biomolecule suitable for modification;
obtaining at least one structure of the at least one biomolecule;
simulating the molecular dynamics of the at least one structure to generate dynamic information about at least one position within the at least one structure;
using the dynamic information to calculate a score for the at least one position;
comparing the score with at least one reference score to identify at least one target position within the biomolecule suitable for modification; and
modifying the at least one target position.

2. The process of claim 1 wherein the at least one biomolecule comprises a polypeptide.

3. The process of claim 1 wherein the at least one biomolecule comprises a nucleic acid.

4. The process of claim 1 wherein the at least one biomolecule comprises a lipid.

5. The process of claim 1 wherein the at least one biomolecule comprises a carbohydrate.

6. The process of claim 1 wherein the modifying of the at least one target position comprises the addition of a reporter group.

7. (canceled)

8. (canceled)

9. The process of claim 1 wherein the modifying of the at least one target position comprises the addition of a linker.

10. The process of claim 1 wherein the modifying of the at least one target position comprises an intramolecular modification selected from the group consisting of at least one addition, at least one deletion, and at least one substitution.

11. The process of claim 10 wherein the intramolecular modification results in the introduction of at least one cysteine residue.

12. The process of claim 1 wherein the at least one structure comprises a three-dimensional representation of the at least one biomolecule in an apo configuration.

13. The process of claim 1 wherein the at least one structure comprises a three-dimensional representation of the at least one biomolecule in a ligand-bound configuration.

14. (canceled)

15. (canceled)

16. The process of claim 1 wherein the score for the at least one position is compared to a reference score for at least one other position within the at least one structure.

17. The process of claim 1 wherein the score for the at least one position is compared to a reference score that is pre-determined.

18. A modified biomolecule designed and produced by the process of claim 1, wherein the modified biomolecule is a biosensor that comprises at least one reporter group.

19. The modified biomolecule of claim 18, wherein the biosensor is for maltooligosaccharides and wherein the biosensor comprises Streptococcus pneumoniae (S. pneumoniae) MalX polypeptide.

20.-24. (canceled)

25. The modified biomolecule of claim 18, wherein the biosensor is for homogalacturonan breakdown products and wherein the biosensor comprises a Yersinia enterocolitica (Y. enterocolitica) TogB polypeptide.

26.-31. (canceled)

32. The modified biomolecule of claim 18, wherein the biosensor is for observing conformational changes and wherein the biosensor comprises an Escherichia coli (E. coli) EF-Tu polypeptide.

33.-41. (canceled)

42. A biosensor for maltooligosaccharides that comprises a Streptococcus pneumoniae (S. pneumoniae) MalX polypeptide and at least one reporter group.

43.-52. (canceled)

53. A biosensor for homogalacturonan breakdown products that comprises a Yersinia enterocolitica (Y. enterocolitica) TogB polypeptide and at least one reporter group.

54.-63. (canceled)

64. A biosensor for observing conformational changes that comprises an Escherichia coli (E. coli) EF-Tu polypeptide and at least one reporter group.

65.-73. (canceled)


SEQUENCE LISTING (3 sequences)

SEQ ID NO: 1 - protein, 416 aa, Streptococcus pneumoniae
  MISC_FEATURE: Unmodified MalX
MGSSHHHHHHSSGLVPRGSHMASDKPADSGSSEVKELTVYVDEGYKSYIEEVAKAYEKEAGVKVTLKTGDALGGLDKLSLDNQNGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETLKQKFGE

SEQ ID NO: 2 - protein, 425 aa, Yersinia enterocolitica
  MISC_FEATURE: Unmodified TogB
MGSSHHHHHHSSGLVPRGSHMEVNLRMSWWGGNGRHQVTLKALEEFHKQHPNINVKAEYTGWDGHLSRLTTQIAGGTEPDVMQTNWNWLPIFSKDGTGFYNLFSVKEQLDLAQFDPKELQQTTVNGKLNGIPISVTARIFYFNDATWAKAGLEYPKTWDELLAAGKVFKEKLGDQYYPVVLEHQDTLALIRSYMTQKYNIPTIDEANKKFAYSPEQWVEFFTMYKTMVDNHVMPSTKYYASFGKSNMYEMKPWINGEWAGTYMWNSTITKYSDNLTKPAKLVLGPYPMLPGAKDAGLFFKPAQMLSIGKSTKHPQESAMLINFLLNSKEGVEALGLERGVPLSATAVTQLRASGVIKDEDPSVAGLNMALELPHKMTTSPYFDDPQIVSLFGDAIQYIDYGQKTVQETAEYFNKQGDRILKRAMR

SEQ ID NO: 3 - protein, 401 aa, Escherichia coli
  MISC_FEATURE: Unmodified EF-Tu
MSKEKFERTKPHVNVGTIGHVDHGKTTLTAAITTVLAKTYGGAARAFDQIDNAPEEKARGITINTSHVEYDTPTRHYAHVDCPGHADYVKNMITGAAQMDGAILVVAATDGPMPQTREHILLGRQVGVPYIIVFLNKCDMVDDEELLELVEMEVRELLSQYDFPGDDTPIVRGSALKALEGDAEWEAKILELAGFLDSYIPEPERAIDKPFLLPIEDVFSISGRGTVVTGRVERGIIKVGEEVEIVGIKETQKSTCTGVEMFRKLLDEGRAGENVGVLLRGIKREEIERGQVLAKPGTIKPHTKFESEVYILSKDEGGRHTPFFKGYRPQFYFRTTDVTGTIELPEGVEMVMPGDNIKMVVTLIHPIAMDDGLRFAIREGGRTVGAGVVAKVLGSHHHHHH